SEQUENCE LISTING (17 sequences)

SEQ ID NO: 1 - protein, 8 aa, synthetic construct
  REGION 1..8: note = CDR-H1 of anti-CEACAM5-antibody
GFVFSSYD

SEQ ID NO: 2 - protein, 8 aa, synthetic construct
  REGION 1..8: note = CDR-H2 of anti-CEACAM5-antibody
ISSGGGIT

SEQ ID NO: 3 - protein, 13 aa, synthetic construct
  REGION 1..13: note = CDR-H3 of anti-CEACAM5-antibody
AAHYFGSSGPFAY

SEQ ID NO: 4 - protein, 6 aa, synthetic construct
  REGION 1..6: note = CDR-L1 of anti-CEACAM5-antibody
ENIFSY

SEQ ID NO: 5 - protein, 9 aa, synthetic construct
  REGION 1..9: note = CDR-L3 of anti-CEACAM5-antibody
QHHYGTPFT

SEQ ID NO: 6 - protein, 120 aa, synthetic construct [sequence omitted]

SEQ ID NO: 7 - protein, 107 aa, synthetic construct [sequence omitted]

SEQ ID NO: 8 - protein, 449 aa, synthetic construct
  REGION 1..449: note = Heavy chain of anti-CEACAM5-antibody
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 214 aa, synthetic construct
  REGION 1..214: note = Light chain of anti-CEACAM5-antibody
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 446 aa, synthetic construct
  REGION 1..446: note = Heavy chain of Ramucirumab
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
  REGION 1..214: note = Light chain of Ramucirumab
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 10 aa, synthetic construct
  REGION 1..10: note = CDR-H1 of anti-VEGFR2-antibody
GFTFSSYSMN

SEQ ID NO: 13 - protein, 17 aa, synthetic construct
  REGION 1..17: note = CDR-H2 of anti-VEGFR2-antibody
SISSSSSYIYYADSVKG

SEQ ID NO: 14 - protein, 7 aa, synthetic construct
  REGION 1..7: note = CDR-H3 of anti-VEGFR2-antibody
VTDAFDI

SEQ ID NO: 15 - protein, 11 aa, synthetic construct
  REGION 1..11: note = CDR-L1 of anti-VEGFR2-antibody
RASQGIDNWLG

SEQ ID NO: 16 - protein, 7 aa, synthetic construct
  REGION 1..7: note = CDR-L2 of anti-VEGFR2-antibody
DASNLDT

SEQ ID NO: 17 - protein, 9 aa, synthetic construct
  REGION 1..9: note = CDR-L3 of anti-VEGFR2-antibody
QQAKAFPPT

(19) United States
(12) Patent Application Publication
(10) Pub. No.: US 2026/0158155 A1
(43) Pub. Date: Jun. 11, 2026

(54) ANTITUMOR COMBINATIONS CONTAINING ANTI-CEACAM5 ANTIBODY-DRUG CONJUGATES AND ANTI-VEGFR-2 ANTIBODIES

(71) Applicants: SANOFI, Paris (FR); ELI LILLY AND COMPANY, Indianapolis, IN (US)

(72) Inventors: Mustapha CHADJAA, Paris (FR); Anne-Sophie LACOSTE-BOURGEACQ, Paris (FR); Nathalie LE BAIL, Paris (FR); Celine NICOLAZZI, Paris (FR); Samira BENSFIA, Paris (FR)

(73) Assignees: SANOFI, Paris, FR (FR); ELI LILLY AND COMPANY, Indianopolis, IN (US)

(21) Appl. No.: 18/707,362

(22) PCT Filed: Nov. 4, 2022
(86) PCT No.: PCT/EP2022/080776

§ 371 Date: May 3, 2024

Related U.S. Application Data
(60) Provisional application No. 63/381,707, filed on Oct. 31, 2022.

(30) Foreign Application Priority Data
Nov. 5, 2021 (EP) 21306552.7

(51) Int. Cl. A61K 47/68; A61K 39/00; A61K 39/395; A61P 35/00; C07K 16/28

(52) U.S. Cl. CPC A61K 47/68033; A61K 39/3955; A61K 47/6853; A61P 35/00; C07K 16/2803; C07K 16/2863; A61K 2039/54; A61K 2039/545

(57) ABSTRACT

The present disclosure concerns antibody-conjugates comprising an anti-CEACAM5-antibody for use for treating cancer in combination with an anti-VEGFR-2 antibody. The disclosure further relates to pharmaceutical compositions and kit-of-parts comprising an anti-CEACAM5-antibody in combination with anti-VEGFR-2 antibody for use for treating cancer.

### TECHNICAL BACKGROUND

The present disclosure concerns antibody-drug conjugates comprising an anti-CEACAM5-antibody for use for treating cancer in combination with an anti-VEGFR-2 antibody. The present disclosure also concerns an anti-VEGFR-2 antibody for use for treating cancer in combination with antibody-drug conjugates comprising an anti-CEACAM5-antibody. The disclosure relates to combination comprising an anti-VEGFR-2 antibody and antibody-drug conjugates comprising an anti-CEACAM5-antibody for use for treating cancer. The disclosure further relates to pharmaceutical compositions and kit-of-parts comprising an anti-CEACAM5-antibody in combination with an anti-VEGFR-2 antibody for use for treating cancer.

Carcino-embryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7 and CEACAM8. Numerous studies have shown that CEACAM5, identical to the originally identified CEA, is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarström et al, 2002, in “Tumor markers, Physiology, Pathobiology, Technology and Clinical Applications” Eds. Diamandis E. P. et al., AACC Press, Washington pp 375). Thus, CEACAM5 may constitute a therapeutic target suitable for tumor specific targeting approaches, such as antibody-drug conjugates (ADCs).

The extracellular domains of CEACAM family members are composed of repeated immunoglobulin-like (Ig-like) domains which have been categorized in 3 types, A, B and N, according to sequence homologies. CEACAM5 contains seven such domains, namely N, A1, B1, A2, B2, A3 and B3. CEACAM5 A1, A2 and A3 domains, on one hand, and B1, B2 and B3 domains, on the other hand, show high sequence homologies, the A domains of human CEACAM5 presenting from 84 to 87% pairwise sequence similarity, and the B domains from 69 to 80%. Furthermore, other human CEACAM members presenting A and/or B domains in their structure, namely CEACAM1, CEACAM6, CEACAM7 and CEACAM8, show homology with human CEACAM5. In particular, the A and B domains of human CEACAM6 protein display sequence homologies with A1 and A3 domains, and any of B1 to B3 domains of human CEACAM5, respectively, which are even higher than observed among the A domains and the B domains of human CEACAM5.

Numerous anti-CEA antibodies were generated in view of CEA-targeted diagnostic or therapeutic purposes. Specificity towards related antigens has always been mentioned as a concern in this field, as an example by Sharkey et al (1990, Cancer Research 50, 2823). Due to the above-mentioned homologies some of previously described antibodies may demonstrate binding to repetitive epitopes of CEACAM5 present in the different immunoglobulin domains and/or show cross-reactivity to other CEACAM members such as CEACAM1, CEACAM6, CEACAM7, or CEACAM8, lacking specificity to CEACAM5. The specificity of the anti-CEACAM5 antibody is desired in view of CEA-targeted therapies such that it binds to human CEACAM5-expressing tumor cells but does not bind to some normal tissues expressing the others CEACAM members.

In the international patent application published as WO 2014/079886 has disclosed an antibody binding to the A3-B3 domain of human and Macaca fascicularis CEACAM5 proteins and which does not significantly cross-react with human CEACAM1, human CEACAM6, human CEACAM7, human CEACAM8, Macaca fascicularis CEACAM1, Macaca fascicularis CEACAM6, and Macaca fascicularis CEACAM8. This antibody has been conjugated to a maytansinoid, thereby providing the antibody-drug conjugate having a significant cytotoxic activity on MKN45 human gastric cancer cells, with IC₅₀ values ≤1 nM.

Antibody-drug conjugates (ADCs) comprise an antibody attached to a chemotherapeutic agent such as a cytotoxic agent or a growth inhibitory agent or a cytostatic agent.

According to an embodiment, the chemotherapeutic agent is attached to the antibody via a chemical linker. These antibody-drug conjugates (ADCs) have great potential in cancer chemotherapy and enable selective delivery of a potent chemotherapeutic agent to target cancer cells, resulting in improved efficacy, reduced systemic toxicity, and improved pharmacokinetics, pharmacodynamics and biodistribution compared to traditional chemotherapy. To date, hundreds of diverse antibody-drug conjugates (ADCs) have been developed against various cancers, of which several have been approved for human use.

The Vascular Endothelial Growth Factor (VEGF) is regarded as the key pro-angiogenic factor that drives tumor angiogenesis. VEGF expression is highly deregulated in primary tumors and in metastatic lesions. In tumors, VEGF is expressed at high levels and by a multiplicity of cell types including cancer cells, tumor stroma and invading myeloid cells leading to endothelial cells hyperproliferation and loss of the guidance mechanisms of angiogenic sprouting.

The activation of Vascular Endothelial Growth Factor Receptor 2 (VEGFR-2) by VEGF is regarded as the most critical driver of tumor angiogenesis. VEGF has been shown to be expressed at high levels in many different types of carcinomas. The auto-phosphorylation of the VEGFR-2 kinase is one of the earliest events upon VEGF binding and is critical for activation of the kinase and subsequent phosphorylation events on the VEGFR-2 receptor. In consequence, antagonist antibodies to VEGFR-2 were produced and studied. In particular, ramucirumab (CAS number 947687-13-0) is a fully human IgG1 monoclonal antibody that binds to the ligand-binding site of VEGFR-2 and prevents its activation. Ramucirumab received approval for use as a monotherapy for hepatocellular carcinoma; as a monotherapy and in combination with chemotherapy in metastatic gastric, gastroesophageal junction adenocarcinoma (GEJ) or metastatic colorectal cancer; in combination with chemotherapy for metastatic non-small cell lung cancer.

However, according to the World Health Organization, cancer was the second leading cause of death globally and responsible for approx. 9.6 million in 2018. Thus, there is continued need for providing improved drug combinations and regimens for the treatment of cancer.

### SUMMARY OF THE DISCLOSURE

The present disclosure relates to an antibody-drug conjugate (ADC) comprising an anti-CEACAM5-antibody conjugated to the cytotoxic maytansinoid agent, (DM4) which is for use in combination with an anti-VEGFR2 antibody for the treatment of cancer.

The present disclosure relates to an antibody-drug conjugate (ADC) comprising an anti-CEACAM5-antibody and a cytotoxic agent which is for use in combination with an anti-VEGFR-2 antibody for the treatment of cancer.

The present disclosure relates to an anti-VEGFR-2 antibody which is for use in combination with an antibody-drug conjugate (ADC) comprising an anti-CEACAM5-antibody and a cytotoxic agent for the treatment of cancer.

The present disclosure further relates to a pharmaceutical composition comprising the antibody-drug conjugate (ADC) comprising an anti-CEACAM5-antibody and a cytotoxic agent and/or an anti-VEGFR-2 antibody, and further the use of the pharmaceutical composition for the treatment of cancer.

The present disclosure further relates to a pharmaceutical composition comprising the antibody-drug conjugate (ADC) comprising an anti-CEACAM5-antibody and a cytotoxic agent and/or an anti-VEGFR-2 antibody, and a pharmaceutically acceptable excipient, and further the use of the pharmaceutical composition for the treatment of cancer.

The present disclosure also relates to a kit comprising (i) a pharmaceutical composition comprising an antibody-drug conjugate (ADC) comprising an anti-CEACAM5-antibody and a cytotoxic agent and (ii) a pharmaceutical composition comprising an anti-VEGFR-2 antibody, in separate or combined formulations.

The present disclosure also relates to a kit comprising (i) a pharmaceutical composition comprising an antibody-drug conjugate (ADC) comprising an anti-CEACAM5-antibody and a cytotoxic agent and a pharmaceutically acceptable excipient, and (ii) a pharmaceutical composition comprising an anti-VEGFR-2 antibody and a pharmaceutically acceptable excipient, in separate or combined formulations.

The present disclosure also relates to a combination comprising an anti-VEGFR-2 and an antibody-drug conjugate (ADC) comprising an anti-CEACAM5-antibody and a cytotoxic agent for use for treating cancer.

The disclosure further relates to the use of the kit for the treatment of cancer.

The disclosure further relates to the pharmaceutical composition, or the kit disclosed herein for the use for treating cancer.

The present inventors have determined that an antibody-drug conjugate (ADC) comprising an anti-CEACAM5-antibody and a cytotoxic agent administered in combination with an anti-VEGFR-2 antibody shows favorable activity for the treatment of cancer.

Also, as shown in the Examples section, the present inventors have determined that an antibody-drug conjugate (ADC) comprising an anti-CEACAM5-antibody, for example conjugated to a maytansinoid, such as tusamitamab ravtansine (huMAb2-3-SPDB-DM4), and administered in combination with an anti-VEGFR-2 antibody, such as ramucirumab, showed a synergistic activity in the reduction of tumor growth and tumor size compared to the effect achieved with the ADC or the anti-VEGFR-2 antibody used alone.

Furthermore, as shown in the Examples section, the inventors have surprisingly observed that an administration of an antibody-drug conjugate, the ADC comprising an anti-CEACAM5-antibody and a cytotoxic agent, at a dose of about 100 mg/m², about 150 mg/m² or about 170 mg/m² in combination with an anti-VEGFR-2 antibody at a dose of about 8 mg/kg or about 10 mg/kg was particularly well tolerated and efficient for treating a gastric cancer (GC) or a gastroesophageal junction cancer (GEJ), when administered every two weeks.

Also, the inventors have surprisingly observed that an administration of an antibody-drug conjugate, the ADC comprising an anti-CEACAM5-antibody and a cytotoxic agent, at a dose of about 100 mg/m², about 120 mg/m², about 135 mg/m², about 150 mg/m² or about 170 mg/m² in combination with an anti-VEGFR-2 antibody at a dose of about 8 mg/kg or about 10 mg/kg was particularly well tolerated and efficient for treating a gastric cancer (GC) or a gastroesophageal junction cancer (GEJ), when administered every three weeks.

Especially, the inventors have surprisingly observed that an administration of an antibody-drug conjugate, the ADC comprising an anti-CEACAM5-antibody and a cytotoxic agent, at a dose of about 120 mg/m², about 150 mg/m² or about 170 mg/m², i.e., as a loading dose or initial dose, in a first cycle, followed by a dose of about 100 mg/m² or 80 mg/m², i.e., a subsequent dose, in a second cycle, and optionally in at least one subsequent (or additional) cycle, in combination with an anti-VEGFR-2 antibody at a dose of about 8 mg/kg in said first, second and optionally in at least one subsequent (or additional) cycle, was particularly well tolerated and efficient for treating a gastric cancer (GC) or a gastroesophageal junction cancer (GEJ), when the first, second and optional subsequent cycle are lasting 2 weeks, i.e., said administration is carried out every two weeks.

Within the disclosure, the expression “loading dose” intends to refer to a dose of drug used at a start of a treatment to frontload an adequate plasma concentration of the drug that will be subsequently maintained by a subsequent dose. A loading dose is typically higher than a subsequent dose. “Loading dose” is used interchangeably with “initial dose” or “first dose”. A “subsequent dose” intends to refer to a dose of a drug, which is administered on a regular schedule, once a high plasma concentration of the drug has been established through the use of a loading dose, to maintain a plateau of the plasma drug concentration. A subsequent dose is typically lower than a loading dose. “Subsequent dose” is used interchangeably with “second dose”.

The inventors have surprisingly observed that an administration of an antibody-drug conjugate, the ADC comprising an anti-CEACAM5-antibody and a cytotoxic agent, at a dose of 120 mg/m², 135 mg/m², 150 mg/m² or 170 mg/m², in a first cycle, and optionally in at least one subsequent cycle, and of an anti-VEGFR-2 antibody, at a dose of 8 or 10 mg/kg, in said first and optionally subsequent cycle, was particularly well tolerated and efficient or treating a gastric cancer (GC), a gastroesophageal junction (GEJ) cancer or a lung cancer, when the first, and additional cycles are lasting 3 weeks, i.e., said administration is carried out every three weeks.

Also, the inventors have surprisingly observed that an administration of an antibody-drug conjugate, the ADC comprising an anti-CEACAM5-antibody and a cytotoxic agent, at a dose of about 80 mg/m² or about 100 mg/m², and of an anti-VEGFR-2 antibody, at a dose of about 8 mg/kg or about 10 mg/kg, in a first and in a second cycle, and optionally in at least one additional cycle, was particularly well tolerated and efficient for treating a lung cancer, such as a non-small cell lung cancer, such as non-squamous non-small-cell lung cancer (NSQ NSCLC), when the first, second, and additional cycle are lasting 2 weeks, i.e., the administration is carried out every two weeks.

The inventors have surprisingly observed that an administration of an antibody-drug conjugate, the ADC comprising an anti-CEACAM5-antibody and a cytotoxic agent, at a dose of 120 mg/m², 135 mg/m², 150 mg/m², or 170 mg/m², and of an anti-VEGFR-2 antibody, at a dose of about 8 or about 10 mg/kg, in a first and in a second cycle, and optionally in at least one subsequent cycle, was particularly well tolerated and efficient for treating a lung cancer, such as a non-small cell lung cancer, such as non-squamous non-small-cell lung cancer (NSQ NSCLC), when the first, second and subsequent cycle are lasting 3 weeks, i.e., the administration is carried out every three weeks.

In some embodiments, the disclosure relates to an antibody-drug conjugate comprising an anti-CEACAM5-antibody and a cytotoxic agent for use for treating cancer in combination with an anti-VEGFR2 antibody.

The present disclosure relates to a combination comprising an antibody-drug conjugate (ADC) comprising an anti-CEACAM5-antibody and a cytotoxic agent and an anti-VEGFR-2 antibody for use in the treatment of cancer.

The cancer is CEACAM5-expressing cancer. “CEACAM5-expressing cancer” is used interchangeably with “CEACAM5 positive cancer”. A CEACAM5 positive cancer may have a CEACAM5 immunohistochemical intensity ≥2+ in ≥50% of cancer cells. Such cancer may be labelled as high CEACAM5-expressing cancer. A CEACAM5 positive cancer may have a CEACAM5 immunohistochemical intensity ≥2+ in ≥1% and <50% of cancer cells. Such cancer may be labelled as moderate CEACAM5-expressing cancer.

A cancer having a CEACAM5 immunohistochemical intensity ≥2+ in <1% of cancer cells is a low or negative CEACAM5-expressing cancer.

In some embodiments, the anti-CEACAM5-antibody may comprise a CDR-H1 consisting of SEQ ID NO: 1, CDR-H2 consisting of SEQ ID NO: 2, CDR-H3 consisting of SEQ ID NO: 3, CDR-L1 consisting of SEQ ID NO: 4, CDR-L2 consisting of amino acid sequence NTR, and CDR-L3 consisting of SEQ ID NO: 5.

In some embodiments, the anti-CEACAM5-antibody may comprise a variable domain of a heavy chain (VH) consisting of SEQ ID NO: 6 and a variable domain of a light chain (VL) consisting of SEQ ID NO: 7.

In some embodiments, the anti-CEACAM5-antibody may comprise a heavy chain (VH) consisting of SEQ ID NO: 8 and a light chain (VL) consisting of SEQ ID NO: 9.

In some embodiments, the antibody-drug conjugate may comprise at least one and at least one cytotoxic agent (also referred to as a chemotherapeutic agent).

In some embodiments, the chemotherapeutic agent may be selected from the group consisting of radioisotopes, protein toxins, small molecule toxins, and combinations thereof.

In some embodiments, the small molecule toxins may be selected from anti-metabolites, DNA-alkylating agents, DNA-cross-linking agents, DNA-intercalating agents, anti-microtubule agents, topoisomerase inhibitors, and combinations thereof.

In some embodiments, the chemotherapeutic agent may be selected from the group consisting of taxanes, vinca alkaloids, maytansinoids, colchicine, podophyllotoxin, gruseofulvin, and combinations thereof.

In some embodiments, the maytansinoids may be selected from the group consisting of N2′-deacetyl-N2′-(3-mercapto-1-oxopropyl)-maytansine (DM1) or N2′-deacetyl-N-2′(4-methyl-4-mercapto-1-oxopentyl)-maytansine (DM4), and combinations thereof.

In some embodiments, the anti-CEACAM5-antibody may be covalently attached via a cleavable or non-cleavable linker to the at least one chemotherapeutic agent.

In some embodiments, the linker may be selected from the group consisting of N-succinimidyl pyridyldithiobutyrate (SPDB), 4-(7yridine-2-yldisulfanyl)-2-sulfo-butyric acid (sulfo-SPDB), and succinimidyl(N-maleimidomethyl) cyclohexane-1-carboxylate (SMCC).

In some embodiments, the CEACAM5-antibody may comprise a heavy chain (VH) consisting of SEQ ID NO: 8 and a light chain (VL) consisting of SEQ ID NO: 9 (huMAb2-3), and which is covalently linked to N2′-deacetyl-N-2′(4-methyl-4-mercapto-1-oxopentyl)-maytansine (DM4) via N-succinimidyl pyridyldithiobutyrate (SPDB).

In the following of the description, the antibody-drug conjugate comprising an anti-CEACAM5-antibody and a cytotoxic agent (also referred to as a chemotherapeutic agent) will be referred to as “the antibody-drug conjugate” or as “the antibody-drug conjugate comprising a CEACAM5-antibody”.

In some embodiments, the antibody-drug conjugate comprising an anti-CEACAM5-antibody and a cytotoxic agent and the anti-VEGFR2 antibody may be administered separately or sequentially to a patient in need thereof.

A patient in need thereof is a patient having a CEACAM5-expressing cancer.

In some embodiments, the antibody-drug conjugate comprising an anti-CEACAM5-antibody and a cytotoxic agent and the anti-VEGFR2 antibody may be formulated in a single pharmaceutical composition comprising the antibody-drug conjugate and the anti-VEGFR2 antibody.

In some embodiments, the antibody-drug conjugate and the anti-VEGFR2 antibody may be formulated in the form of two separate pharmaceutical compositions, wherein (i) one pharmaceutical composition may comprise the antibody-drug conjugate, and (ii) the other pharmaceutical composition may comprise the anti-VEGFR2 antibody.

The antibody-drug conjugate and the anti-VEGFR-2 antibody may be simultaneously, separately, or sequentially administered.

In some embodiments, the antibody-drug conjugate and the anti-VEGFR2 antibody may be administered separately or sequentially to a patient in need thereof.

Withing the disclosure, the expression “sequentially administered” when used with reference to the administration of at least two drugs intends to mean that a second drug is administered subsequently in time to the first drug, that is one drug is administered before or after the other drug. The administrations may be carried out by a same route or by distinct routes. The period of time between the administration of the first drug and the administration of the second drug may last from about 5 minutes to about 3 hours, for example from 10 minutes to about 2.5 hours, from about 30 minutes to about 2 hours, or from about 1 hour to about 1.5 hours. A period of time between the administration of the first drug and the administration of the second drug may last about 5 minutes, about 10 minutes, about 30 minutes, about 1 hour, 1.5 hours, about 2 hours, about 2.5 hours or about 3 hours.

Withing the disclosure, the expression “simultaneously administered” when used with reference to the administration of at least two drugs intends to mean that the first and second drug are administered at the same time, or concurrently, and possibly by the same route, such as for example, when formulated in the same composition.

Withing the disclosure, the expression “separately administered” when used with reference to the administration of at least two drugs intends to mean that the first and second drug are administered by separate routes or by the same route but at different location of the body, e.g., two intramuscular administrations in different muscles. The administration may be carried out simultaneously in time or sequentially. Usually, the administrations are carried concomitantly, that is in a timeframe usually of less than about 5 minutes.

In some embodiments, the antibody-drug conjugate and the anti-VEGFR-2 antibody are sequentially administered.

The antibody-drug conjugate may be administered before or after the anti-VEGFR-2 antibody. The antibody-drug conjugate may be administered after, i.e., subsequently to, the anti-VEGFR-2 antibody.

In some embodiments, the antibody-drug conjugate may be administered after the anti-VEGFR-2 antibody.

In some embodiments, the antibody-drug conjugate and the anti-VEGFR-2 antibody may be administered for at least one cycle of treatment.

Withing the disclosure, the expression “cycle of treatment” intends to refer to a period of treatment followed by a period of rest (no treatment) that is repeated on a regular schedule. For example, treatment given for one day followed by one or more days of rest is one treatment cycle. When this cycle is repeated multiple times on a regular schedule, it makes up a course of treatment. For example, within the disclosure, a cycle of treatment may extend over a period of 1, 2, 3, 4, 5 or 6 weeks, with day one of the cycle being the period of treatment and the following days being the period of rest. A treatment (or course of treatment) may comprise a first cycle followed by at least a second cycle, and optionally at least one additional cycle. A treatment may comprise 2, 3, 4, 5, 6, 7, 8, 9, 10 or more cycles.

In some embodiments, the antibody-drug conjugate and the anti-VEGFR-2 antibody may be administered for a first cycle of treatment, and to at least one additional cycle of treatment.

In some embodiments, the antibody-drug conjugate and the anti-VEGFR-2 antibody may be administered at day 1 of a first cycle of treatment.

In some embodiments, the antibody-drug conjugate and the anti-VEGFR-2 antibody may be administered at day 1 of at least one additional (or subsequent) cycle of treatment.

In some embodiments, a cycle of treatment may be about two or three weeks.

In some embodiments, a cycle of treatment may be about two weeks.

In some embodiments, a cycle of treatment may be about three weeks.

In some embodiments, the cancer may be a CEACAM5-expressing cancer.

In some embodiments, the cancer may be a CEACAM5 positive cancer having a CEACAM5 immunohistochemical intensity ≥2+ in ≥50% of cancer cells as measured by immunohistochemistry. Such CEACAM5 positive or CEACAM5-expressing cancer may be labelled high CEACAM5 positive cancer.

In some embodiments, the cancer may be a CEACAM5 positive cancer having a CEACAM5 immunohistochemical intensity ≥2+ intensity in ≥1% and <50% of cancer cells as measured by immunohistochemistry. Such CEACAM5 positive or CEACAM5-expressing cancer may be labelled moderate CEACAM5 positive cancer.

In some embodiments, the cancer may be selected from hepatocellular carcinoma, colorectal cancer, gastric cancer, gastroesophageal junction (GEJ) adenocarcinoma, lung cancer, uterus cervix cancer, pancreas cancer, ovary cancer, thyroid cancer, bladder cancer, endometrium cancer, breast cancer, liver cancer (for instance cholangiocarcinoma), prostate cancer and skin cancer.

In some embodiments, the cancer may be selected from gastric cancer, gastroesophageal junction (GEJ) adenocarcinoma, and lung cancer, such as non-squamous non-small cell lung cancer.

In some embodiments, the cancer is gastric (GC) or gastroesophageal adenocarcinoma (GEJ cancer).

In some embodiments, the lung cancer may be a non-squamous non-small-cell lung cancer (NSQ NSCLC).

In some embodiments, the disclosure relates to an antibody-drug conjugate comprising an anti-CEACAM5-antibody and a cytotoxic agent for use for treating a cancer in combination with an anti-VEGFR2 antibody, wherein the antibody-drug conjugate may be administered at a dose of 60 mg/m² to 210 mg/m² and the anti-VEGFR-2 antibody may be administered at a dose of 2 mg/kg to 20 mg/kg.

In some embodiments, the disclosure relates to an antibody-drug conjugate comprising an anti-CEACAM5-antibody and a cytotoxic agent for use for treating a cancer in combination with an anti-VEGFR2 antibody, wherein the antibody-drug conjugate may be administered at a dose of about 60 mg/m² to about 210 mg/m².

The dose may be based on the body surface area of the patient. In some embodiments, for patients having a body surface area (BSA) above 2.2 m², the dosage of antibody-drug conjugate may be capped on the basis of a BSA of 2.2 m².

The antibody-drug conjugate may be administered at a dose of about 60, 80, 100, 120, 135, 150, 170, 180, 190 or about 210 mg/m².

The antibody-drug conjugate may be administered at a dose of about 80 mg/m² to about 170 mg/m².

The antibody-drug conjugate may be administered at a dose of about 80 mg/m², about 100 mg/m², about 120 mg/m², about 150 mg/m² or about 170 mg/m².

The antibody-drug conjugate may be administered at a dose of about 120, 135, 150 or about 170 mg/m².

The antibody-drug conjugate may be administered at a dose of about 80, 100, 120, 135, 150 or about 170 mg/m² as a loading—or first—dose.

The antibody-drug conjugate may be administered at a dose of about 80, 100, 120, 135, 150 or about 170 mg/m² as a subsequent—or second—dose.

The antibody-drug conjugate may be administered at a dose of about 80 mg/m², about 100 mg/m² as a subsequent dose.

In some embodiments, at day 1 of a first cycle of treatment the antibody-drug conjugate may be administered at a dose of 80 mg/m², 100 mg/m², 120 mg/m², 135 mg/m², 150 mg/m² or 170 mg/m².

In some embodiments, at day 1 of a first cycle of treatment, the antibody-drug conjugate may be administered at a dose of about 120 mg/m², 135 mg/m² or 150 mg/m².

In some embodiments, at day 1 of a first cycle of treatment, the antibody-drug conjugate may be administered at a dose of about 80 mg/m² or about 100 mg/m².

In some embodiments, the disclosure relates to an antibody-drug conjugate comprising an anti-CEACAM5-antibody and a cytotoxic agent for use for treating a cancer in combination with an anti-VEGFR2 antibody, wherein the anti-VEGFR-2 antibody may be administered at a dose of about 2 mg/kg to about 20 mg/kg.

The anti-VEGFR-2 antibody may be administered at a dose of from about 2 to about 20 mg/kg, or from about 4 to about 15 mg/kg, or from about 6 to about 10 mg/kg, or at about 8 mg/kg.

The anti-VEGFR-2 antibody may be administered at a dose of about 8 mg/kg or about 10 mg/kg.

The anti-VEGFR-2 antibody may be administered at a dose of about 8 mg/kg.

The anti-VEGFR-2 antibody may be administered at a dose of about 10 mg/kg.

In some embodiments, at day 1 of a first cycle of treatment the antibody-drug conjugate may be administered at a dose of 80 mg/m², 100 mg/m², 150 mg/m² or 170 mg/m², and the cycle of treatment may be 2 weeks.

In some embodiments, at day 1 of a first cycle of treatment the antibody-drug conjugate may be administered at a dose of 80 mg/m², 100 mg/m², 120 mg/m², 135 mg/m², 150 mg/m² or 170 mg/m², and the cycle of treatment may be 3 weeks.

In some embodiments, at day 1 of a first cycle of treatment the antibody-drug conjugate may be administered at a dose of 120 mg/m², 135 mg/m², 150 mg/m² or 170 mg/m², and the cycle of treatment may be 3 weeks.

In an additional (or subsequent) cycle, the antibody-drug conjugate may be administered at a dose of about 100 mg/m².

In some embodiments, at day 1 of an additional cycle of treatment (at least one cycle subsequent to the first cycle), the antibody-drug conjugate may be administered at a dose of 80 mg/m², 100 mg/m², 120 mg/m², 135 mg/m², 150 mg/m² or 170 mg/m².

In some embodiments, at day 1 of an additional cycle of treatment (at least one cycle subsequent to the first cycle), the antibody-drug conjugate may be administered at a dose of about 100 mg/m².

In some embodiments, at day 1 of an additional cycle of treatment (at least one cycle subsequent to the first cycle), the antibody-drug conjugate may be administered at a dose of about 80 mg/m².

The antibody-drug conjugate may be administered at a dose of about 80 mg/m², 100 mg/m², 120 mg/m², 135 mg/m², 150 mg/m² or about 170 mg/m² at day 1 of a first cycle of treatment and at a dose of about 80 mg/m², 100 mg/m², 120 mg/m², 135 mg/m², 150 mg/m² or about 170 mg/m² at day 1 of an additional cycle of treatment. A cycle may be 2 or 3 weeks.

The antibody-drug conjugate may be administered at a dose of about 80 mg/m² or 100 mg/m² at day 1 of a first cycle of treatment and at a dose of about 80 mg/m² or 100 mg/m² at day 1 of an additional cycle of treatment. A cycle may be 2 weeks.

The antibody-drug conjugate may be administered at a dose of about 120 mg/m², 135 mg/m², 150 mg/m² or about 170 mg/m² at day 1 of a first cycle of treatment and at a dose of about 120 mg/m², 135 mg/m², 150 mg/m² or about 170 mg/m² at day 1 of an additional cycle of treatment. A cycle may be 3 weeks.

The antibody-drug conjugate may be administered at a dose of about 120 mg/m², 135 mg/m², 150 mg/m² or about 170 mg/m² at day 1 of a first cycle of treatment and at a dose of about 80 mg/m² or about 100 mg/m² at day 1 of an additional cycle of treatment.

The antibody-drug conjugate may be administered at a dose of about 120 mg/m², 135 mg/m² or 150 mg/m² at day 1 of a first cycle of treatment and at a dose of about 100 mg/m² at day 1 of an additional cycle of treatment.

The antibody-drug conjugate may be administered at a dose of about 150 mg/m² or about 170 mg/m² at day 1 of a first cycle of treatment and at a dose of about 80 mg/m² or about 100 mg/m² at day 1 of an additional cycle of treatment. A cycle may be 2 weeks.

The antibody-drug conjugate may be administered at a dose of about 100 mg/m² or about 80 mg/m² at day 1 of a first cycle of treatment and at a dose of about 100 mg/m² at day 1 of an additional cycle.

In some embodiments, at day 1 of a first cycle of treatment, the anti-VEGFR-2 antibody may be administered at a dose of about 8 mg/kg or about 10 mg/kg.

In some embodiments, at day 1 of a first cycle of treatment, the anti-VEGFR-2 antibody may be administered at a dose of about 8 mg/kg.

In some embodiments, at day 1 of a first cycle of treatment, the anti-VEGFR-2 antibody may be administered at a dose of about 10 mg/kg.

The anti-VEGFR-2 antibody may be administered at a dose from 8 mg/kg, and the cycle of treatment may be 2 weeks.

The anti-VEGFR-2 antibody may be administered at a dose from 10 mg/kg, and the cycle of treatment may be 3 weeks.

In an additional (or subsequent) cycle, the anti-VEGFR-2 antibody may be administered at a dose of about 8 mg/kg or about 10 mg/kg.

In some embodiments, at day 1 of an additional cycle of treatment (at least one cycle subsequent to the first cycle), the anti-VEGFR-2 antibody may be administered at a dose of about 8 mg/kg or about 10 mg/kg

In some embodiments, at day 1 of an additional cycle of treatment (at least one cycle subsequent to the first cycle), the anti-VEGFR-2 antibody may be administered at a dose of about 8 mg/kg.

In some embodiments, at day 1 of an additional cycle of treatment (at least one cycle subsequent to the first cycle), the anti-VEGFR-2 antibody may be administered at a dose of about 10 mg/kg.

In some embodiments, at day 1 of a first cycle of treatment and at day 1 of an additional cycle of treatment (at least one cycle subsequent to the first cycle), the anti-VEGFR-2 antibody may be administered at a dose of about 8 mg/kg. A cycle may be 2 weeks.

In some embodiments, at day 1 of a first cycle of treatment and at day 1 of an additional cycle of treatment (at least one cycle subsequent to the first cycle), the anti-VEGFR-2 antibody may be administered at a dose of about 10 mg/kg. A cycle may be 3 weeks.

In some embodiments, the cancer may be a lung cancer.

In some embodiments, the cancer may be a lung cancer and the antibody-drug conjugate may be administered at a dose of 80 mg/m² to 170 mg/m².

In some embodiments, the cancer may be a lung cancer, such as a non-small cell lung cancer, such as non-squamous non-small-cell lung cancer (NSQ NSCLC), and the antibody-drug conjugate may be administered at a dose of about 80 mg/m² to about 170 mg/m².

The anti-VEGFR-2 antibody may be administered at a dose of about 8 mg/kg to about 10 mg/kg.

In some embodiments, the cancer may be a lung cancer, such as a non-small cell lung cancer, such as non-squamous non-small-cell lung cancer (NSQ NSCLC), and the antibody-drug conjugate may be administered at a dose of 80 mg/m², 100 mg/m², 120 mg/m², 135 mg/m², 150 mg/m² or 170 mg/m².

The lung cancer may be a non-squamous non-small-cell lung cancer (NSQ NSCLC).

In some embodiments, at day 1 of a first cycle of treatment, the antibody-drug conjugate may be administered at a dose of 80 mg/m², 100 mg/m², 120 mg/m² or 150 mg/m².

In some embodiments, at day 1 of a first cycle of treatment, the antibody-drug conjugate may be administered at a dose of 80 mg/m² or 100 mg/m². The anti-VEGFR-2 antibody may be administered at a dose of 8 mg/kg.

In some embodiments, at day 1 of a first cycle of treatment, the antibody-drug conjugate may be administered at a dose of 80 mg/m² or 100 mg/m², and the cycle of treatment may be 2 weeks.

In some embodiments, at day 1 of a first cycle of treatment, the anti-VEGFR-2 antibody may be administered at a dose of 8 mg/kg, and the cycle may be 2 weeks

In some embodiments, at day 1 of a first cycle of treatment, the antibody-drug conjugate may be administered at a dose of about 80 mg/m². The anti-VEGFR-2 antibody may be administered at a dose of about 8 mg/kg. The cycle of treatment may last 2 weeks.

In some embodiments, at day 1 of a first cycle of treatment, the antibody-drug conjugate may be administered at a dose of about 100 mg/m². The anti-VEGFR-2 antibody may be administered at a dose of about 8 mg/kg. The cycle of treatment may last 2 weeks.

In some embodiments, at day 1 of a first cycle of treatment, the antibody-drug conjugate may be administered at a dose of 120 mg/m², 135 mg/m², 150 mg/m² or 170 mg/m² and the cycle of treatment may last 3 weeks. The anti-VEGFR-2 antibody may be administered at a dose of about 10 mg/kg.

In some embodiments, at day 1 of a first cycle of treatment, the antibody-drug conjugate may be administered at a dose of 120 mg/m² or 150 mg/m², and the cycle of treatment may be 3 weeks.

In some embodiments, at day 1 of a first cycle of treatment the anti-VEGFR-2 antibody may be administered at a dose of 10 mg/kg, and the cycle may be 3 weeks.

In some embodiments, at day 1 of an at least one additional cycle of treatment, the antibody-drug conjugate may be administered at a dose of 80 mg/m², 100 mg/m², 120 mg/m² or 150 mg/m².

In some embodiments, at day 1 of an at least one additional cycle of treatment the antibody-drug conjugate may be administered at a dose of 80 mg/m² or 100 mg/m², and the cycle may be 2 weeks.

In some embodiments, at day 1 of an at least one additional (or subsequent) cycle of treatment, the antibody-drug conjugate may be administered at a dose of 80 mg/m². The cycle of treatment may last 2 weeks.

In some embodiments, at day 1 of an at least one additional (or subsequent) cycle of treatment, the antibody-drug conjugate may be administered at a dose of 100 mg/m². The cycle of treatment may last 2 weeks.

In some embodiments, at day 1 of an at least one additional cycle of treatment the anti-VEGFR-2 antibody may be administered at a dose of 8 mg/kg, and the cycle may be 2 weeks.

In some embodiments, at day 1 of an at least one additional cycle of treatment the antibody-drug conjugate may be administered at a dose of 120 mg/m² or 150 mg/m², and the cycle last 3 weeks.

In some embodiments, at day 1 of an at least one additional cycle of treatment, the antibody-drug conjugate may be administered at a dose of 170 mg/m².

In some embodiments, at day 1 of an at least one additional cycle of treatment the anti-VEGFR-2 antibody may be administered at a dose of 10 mg/kg, and the cycle may be 3 weeks.

In some embodiments, at day 1 of a first cycle and at day 1 of an at least one additional cycle of treatment, the antibody-drug conjugate may be administered at a dose of 80 mg/m² and the anti-VEGFR-2 antibody may be administered at a dose of 8 mg/kg, and the cycle of treatment may be 2 weeks.

In some embodiments, at day 1 of a first cycle and at day 1 of an at least one additional cycle of treatment, the antibody-drug conjugate may be administered at a dose of 100 mg/m² and the anti-VEGFR-2 antibody may be administered at a dose of 8 mg/kg, and the cycle of treatment may be 2 weeks.

In some embodiments, at day 1 of a first cycle and at day 1 of an at least one additional cycle of treatment, the antibody-drug conjugate may be administered at a dose of 120 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 150 mg/m² and the anti-VEGFR-2 antibody may be administered at a dose of 10 mg/kg, and the cycle of treatment may be 3 weeks.

In some embodiments, the cancer may be a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg and subsequently the antibody-drug conjugate comprising an anti-CEACAM5-antibody may be administered at a dose of 80 mg/m², on day 1 of a first cycle, and the cycle may be 2 weeks.

In some embodiments, the anti-VEGFR-2 antibody may be administered at a dose of 8 mg/kg as a subsequent dose, and subsequently the antibody-drug conjugate comprising an anti-CEACAM5-antibody may be administered at a dose of 80 mg/m² as a subsequent dose, on day 1 of additional cycle(s), and the cycle(s) may be 2 weeks.

In some embodiments, the cancer may be a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg and subsequently the antibody-drug conjugate comprising an anti-CEACAM5-antibody may be administered at a dose of 100 mg/m², on day 1 of a first cycle, and the cycle may be 2 weeks.

In some embodiments, the anti-VEGFR-2 antibody may be administered at a dose of 8 mg/kg as a subsequent dose, and subsequently the antibody-drug conjugate comprising an anti-CEACAM5-antibody may be administered at a dose of 100 mg/m² as a subsequent dose, on day 1 of additional cycle(s), and the cycle(s) may be weeks.

In some embodiments, the cancer may be a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg and subsequently the antibody-drug conjugate comprising an anti-CEACAM5-antibody may be administered at a dose of 120 mg/m², on day 1 of a first cycle, and the cycle may be 3 weeks.

In some embodiments, the anti-VEGFR-2 antibody may be administered at a dose of 10 mg/kg as a subsequent dose, and sequentially the antibody-drug conjugate comprising an anti-CEACAM5-antibody may be administered at a dose of 120 mg/m² as a subsequent dose, on day 1 of additional cycle(s), and the cycle(s) may be 3 weeks.

In some embodiments, the cancer may be a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg and subsequently the antibody-drug conjugate comprising an anti-CEACAM5-antibody may be administered at a dose of 150 mg/m², on day 1 of a first cycle, and the cycle may be 3 weeks.

In some embodiments, the anti-VEGFR-2 antibody may be administered at a dose of 10 mg/kg as a subsequent dose, and sequentially the antibody-drug conjugate comprising an anti-CEACAM5-antibody may be administered at a dose of 150 mg/m² as a subsequent dose, on day 1 of additional cycle(s), and the cycle(s) may be 3 weeks.

In some embodiments, the cancer is a gastric cancer or a gastroesophageal adenocarcinoma (GEJ) cancer.

In some embodiments, the cancer is a gastric cancer or a gastroesophageal adenocarcinoma (GEJ) cancer and wherein the antibody-drug conjugate is administered at a dose of 80 mg/m² to 170 mg/m².

In some embodiments, the cancer may be a gastric cancer (GC) or a gastroesophageal adenocarcinoma (GEJ) cancer and the antibody-drug conjugate may be administered at a dose from about 100 mg/m² to about 170 mg/m². The anti-VEGFR-2 antibody may be administered at a dose from about 8 mg/kg to about 10 mg/kg.

In some embodiments, the antibody-drug conjugate may be administered at a dose of 80 mg/m², 100 mg/m², 120 mg/m², 135 mg/m², 150 mg/m² or 170 mg/m².

In some embodiments, at day 1 of a first cycle of treatment the antibody-drug conjugate may be administered at a dose of 80 mg/m², 100 mg/m², 120 mg/m², 135 mg/m², 150 mg/m² or 170 mg/m².

At day 1 of a first cycle of treatment, the antibody-drug conjugate may be administered at a dose of about 120 mg/m², 135 mg/m², about 150 mg/m² or about 170 mg/m². The cycle may be 2 or 3 weeks. The cycle may be 3 weeks.

The anti-VEGFR-2 antibody may be administered at a dose from about 8 mg/kg to about 10 mg/kg.

At day 1 of a first cycle of treatment, the antibody-drug conjugate may be administered at a dose of about 120 mg/m². The anti-VEGFR-2 antibody may be administered at a dose of about 8 mg/kg or about 10 mg/kg. The cycle of treatment may last 2 or 3 weeks.

At day 1 of a first cycle of treatment, the antibody-drug conjugate may be administered at a dose of about 135 mg/m². The anti-VEGFR-2 antibody may be administered at a dose of about 8 mg/kg or about 10 mg/kg. The cycle of treatment may last 2 or 3 weeks.

At day 1 of a first cycle of treatment, the antibody-drug conjugate may be administered at a dose of about 150 mg/m². The anti-VEGFR-2 antibody may be administered at a dose of about 8 mg/kg about 10 mg/kg. The cycle of treatment may last 2 or 3 weeks.

At day 1 of a first cycle of treatment, the antibody-drug conjugate may be administered at a dose of about 170 mg/m². The anti-VEGFR-2 antibody may be administered at a dose of about 8 mg/kg about 10 mg/kg. The cycle of treatment may last 2 or 3 weeks.

In some embodiments, at day 1 of a first cycle of treatment the antibody-drug conjugate is administered at a loading dose of 120 mg/m², 135 mg/m², 150 mg/m² or 170 mg/m², and the cycle is 2 weeks.

In some embodiments, at day 1 of a first cycle of treatment the antibody-drug conjugate is administered at a loading dose of 150 mg/m² or 170 mg/m², and the cycle is 2 weeks.

In some embodiments, at day 1 of an at least one additional cycle of treatment the antibody-drug conjugate is administered at a subsequent dose of 80 mg/m² or 100 mg/m², and the cycle is 2 weeks In some embodiments, at day 1 of additional cycles (at least one cycle subsequent to the first cycle), the antibody-drug conjugate may be administered at a dose of about 100 mg/m². The anti-VEGFR-2 antibody may be administered at a dose of about 8 mg/kg or about 10 mg/kg. The cycle of treatment may last 2 or 3 weeks.

The cycle(s) of treatment may last about 2 weeks. The cycle(s) of treatment may last about 3 weeks.

In some embodiments, the anti-VEGFR2 antibody may be administered at a dose of 8 mg/kg, and the cycle may be 2 weeks.

In some embodiments, the anti-VEGFR2 antibody may be administered at a dose of 10 mg/kg, and the cycle may be 3 weeks.

In some embodiments, at day 1 of a first cycle, the antibody-drug conjugate may be administered at a loading dose of 150 mg/m² and the anti-VEGFR-2 antibody may be administered at a dose of 8 mg/kg, and at day 1 of an at least one additional cycle of treatment the antibody-drug conjugate may be administered at a subsequent dose of 80 mg/m² and the anti-VEGFR-2 antibody may be administered at a dose of 8 mg/kg, and the cycle of treatment may be 2 weeks.

In some embodiments, at day 1 of a first cycle, the antibody-drug conjugate may be administered at a loading dose of 170 mg/m² and the anti-VEGFR-2 antibody may be administered at a dose of 8 mg/kg, and at day 1 of an at least one additional cycle of treatment the antibody-drug conjugate may be administered at a subsequent dose of 100 mg/m² and the anti-VEGFR-2 antibody may be administered at a dose of 8 mg/kg, and the cycle of treatment may be 2 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg, and subsequently the antibody-drug conjugate comprising an anti-CEACAM5-antibody may be administered at a dose of 150 mg/m², on day 1 of a first cycle, and the cycle may be 2 weeks.

In some embodiments, the anti-VEGFR-2 antibody may be administered at a dose of 8 mg/kg as a subsequent dose, and subsequently the antibody-drug conjugate comprising an anti-CEACAM5-antibody may be administered at a dose of 80 mg/m² as a subsequent dose, on day 1 of additional cycle(s), and the cycle(s) may be 2 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity >2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg, and subsequently the antibody-drug conjugate comprising an anti-CEACAM5-antibody may be administered at a dose of 170 mg/m², on day 1 of a first cycle, and the cycle may be 2 weeks.

In some embodiments, the anti-VEGFR-2 antibody may be administered at a dose of 8 mg/kg as a subsequent dose, and subsequently the antibody-drug conjugate comprising an anti-CEACAM5-antibody may be administered at a dose of 100 mg/m² as a subsequent dose, on day 1 of additional cycle(s), and the cycle(s) may be 2 weeks.

In some embodiments, the antibody-drug conjugate may be administered at a dose of 80 mg/m², 100 mg/m², 120 mg/m², 135 mg/m² 150 mg/m² or 170 mg/m², and the cycle may be 3 weeks.

In some embodiments, the antibody-drug conjugate may be administered at a dose of 120 mg/m², 150 mg/m² or 170 mg/m², and the cycle may be 3 weeks.

In some embodiments, the anti-VEGFR2 antibody may be administered at a dose of 10 mg/kg, and the cycle may be 3 weeks.

In some embodiments, at day 1 of a first cycle and at day 1 of an at least one additional cycle of treatment, the antibody-drug conjugate may be administered at a dose of 80 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 100 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 120 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 135 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 150 mg/m² and the anti-VEGFR-2 antibody may be administered at a dose of 10 mg/kg, and the cycle of treatment may be 3 weeks.

In some embodiments, at day 1 of a first cycle and at day 1 of an at least one additional cycle of treatment, the antibody-drug conjugate may be administered at a dose of 170 mg/m² and the anti-VEGFR-2 antibody may be administered at a dose of 10 mg/kg, and the cycle of treatment may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 80 mg/m², 100 mg/m², 120 mg/m², 150 mg/m² or 170 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 80 mg/m², 100 mg/m², 120 mg/m², 150 mg/m² or 170 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 80 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 80 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 100 mg/m², on day 1 of a first cycle, and the cycle may be 3 weeks.

In some embodiments, anti-VEGFR-2 antibody may be administered at a dose of 10 mg/kg as a subsequent dose, and subsequently the antibody-drug conjugate comprising an anti-CEACAM5-antibody may be administered at a dose of 100 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 120 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 120 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 135 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 135 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of c cancer ells, wherein the anti-VEGFR-2 antibody may be administered at a dose of 10 mg/kg, and subsequently the antibody-drug conjugate comprising an anti-CEACAM5-antibody may be administered at a dose of 150 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 150 mg/m² as a subsequent dose, on day 1 of additional cycle(s), and the cycle(s) may be 3 weeks.

In some embodiments, the cancer may be a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 170 mg/m², on day 1 of a first cycle, and the cycle may be 3 weeks.

In some embodiments, the anti-VEGFR-2 antibody may be administered at a dose of 10 mg/kg as a subsequent dose, and subsequently the antibody-drug conjugate comprising an anti-CEACAM5-antibody may be administered at a dose of 170 mg/m² as a subsequent dose, on day 1 of additional cycle(s), and the cycle(s) may be 3 weeks.

In some embodiments, an antibody-drug conjugate (ADC) comprising an anti-CEACAM5-antibody conjugated to a cytotoxic agent, such as a maytansinoid (e.g., DM4), for example tusamitamab ravtansine, in combination with an anti-VEGFR-2 antibody such as ramucirumab, may be used as disclosed herein for achieving a synergistic effect in the treatment of cancer.

The synergistic effect may be achieved in the reduction of tumor growth.

The synergistic effect may be achieved in the reduction of tumor size.

The tumor may be a tumor from a cancer, for example a carcinoma, for example a gastric cancer, a gastroesophageal adenocarcinoma (GEJ) cancer, or a lung cancer, for example a non-squamous non-small cells lung cancer.

The combination as disclosed herein may be used, for example in the different uses and methods disclosed herein, for treating a cancer in achieving a synergistic effect in reduction of tumor size or tumor growth.

The antibody-drug conjugate may be tusamitamab ravtansine (huMAb2-3-SPDB-DM4).

The anti-VEGFR-2 antibody may be ramucirumab.

In some embodiments, the disclosure relates to a pharmaceutical composition comprising the antibody-drug conjugate and an anti-VEGFR2 antibody, as disclosed herein, and a pharmaceutically acceptable excipient.

In some embodiments, the disclosure relates to a pharmaceutical composition comprising the antibody-drug conjugate as disclosed herein, and ramucirumab and a pharmaceutically acceptable excipient.

In some embodiments, the disclosure relates to a pharmaceutical composition comprising tusamitamab ravtansine, and ramucirumab and a pharmaceutically acceptable excipient.

In some embodiments, the disclosure relates to a kit comprising (i) a pharmaceutical composition of the antibody-drug conjugate as disclosed herein and a pharmaceutically acceptable excipient and (ii) a pharmaceutical composition comprising an anti-VEGFR2 antibody and a pharmaceutically acceptable excipient.

In some embodiments, the disclosure relates to a pharmaceutical composition, or the kit, as disclosed herein, for the use for treating cancer

### DETAILED DESCRIPTION OF THE DISCLOSURE

### Definitions

An “antibody” may be a natural or conventional antibody in which two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e., to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. A human framework region is a framework region that is substantially identical (about 85%, or more, in particular 90%, 95%, 97%, 99% or 100%) to the framework region of a naturally occurring human antibody.

In the context of the disclosure, CDR/FR definition in an immunoglobulin light or heavy chain is to be determined based on IMGT definition (Lefranc et al. Dev. Comp. Immunol., 2003, 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanized, bispecific or multispecific antibodies.

As used herein, antibody or immunoglobulin also includes “single domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered single domain antibodies. Single domain antibodies may be derived from any species including, but not limited to mouse, human, camel, llama, goat, rabbit, bovine. Single domain antibodies may be naturally occurring single domain antibodies known as heavy chain antibody devoid of light chains. In particular, camelidae species, for example camel, dromedary, llama, alpaca and guanaco, produce heavy chain antibodies naturally devoid of light chain. Camelid heavy chain antibodies also lack the CH1 domain.

The variable heavy chain of these single domain antibodies devoid of light chains are known in the art as “VHH” or “Nanobody®”. Similar to conventional VH domains, VHHs contain four FRs and three CDRs. VHH have advantages over conventional antibodies: they are about ten times smaller than IgG molecules, and as a consequence properly folded functional VHH can be produced by in vitro expression while achieving high yield. Furthermore, VHH are very stable, and resistant to the action of proteases. The properties and production of VHH have been reviewed by Harmsen and De Haard H J (Appl. Microbiol. Biotechnol. 2007 November; 77(1):13-22).

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e., produced by protein engineering.

The term “humanized antibody” refers to an antibody which is wholly or partially of non-human origin, and which has been modified to replace certain amino acids, in particular in the framework regions of the VH and VL domains, in order to avoid or minimize an immune response in humans. The constant domains of a humanized antibody are most of the time human CH and CL domains.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 and antigen binding activity, in which about a half of the N-terminal side of the heavy chain and the entire light chain are bound together through a disulfide bond. It is usually obtained among fragments by treating IgG with a protease, such as papain.

The term “F(ab′)2” refers to an antibody fragment having a molecular weight of about 100,000 and antigen binding activity, which is slightly larger than 2 identical Fab fragments bound via a disulfide bond of the hinge region. It is usually obtained among fragments by treating IgG with a protease, such as pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. The human scFv fragment of the disclosure includes CDRs that are held in appropriate conformation, in particular by using gene recombination techniques. Divalent and multivalent antibody fragments can form either spontaneously by association of monovalent scFvs, or can be generated by coupling monovalent scFvs by a peptide linker, such as divalent sc(Fv)2. “dsFv” is a VH::VL heterodimer stabilized by a disulphide bond. “(dsFv)2” denotes two dsFv coupled by a peptide linker.

The term “bispecific antibody” or “BsAb” denotes an antibody which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which combines the antigen-binding sites of two or more antibodies within a single molecule.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains of the same chain, the domains are forced to pair with the complementary domains of another chain and create two antigen-binding sites.

An amino acid sequence “at least 85% identical to a reference sequence” is a sequence having, on its entire length, 85%, or more, in particular 90%, 91%, 92%, 93%, 94%, 95%, 96%, 97%, 98% or 99% sequence identity with the entire length of the reference amino acid sequence.

A percentage of “sequence identity” between amino acid sequences may be determined by comparing the two sequences, optimally aligned over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g., using the algorithm of Needleman and Wunsch J. Mol. Biol. 48:443 (1970). The percentage of sequence identity can be readily determined for instance using the program Needle, with the BLOSUM62 matrix, and the following parameters gap-open=10, gap-extend=0.5.

A “conservative amino acid substitution” is one in which an amino acid residue is substituted by another amino acid residue having a side chain R group with similar chemical properties (e.g., charge, size or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups can also be defined on the basis of amino acid size.

By “purified” and “isolated” it is meant, when referring to a polypeptide (i.e., the antibody of the disclosure) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

As used herein, the term “subject” or “patient” denotes a mammal, such as a rodent, a feline, a canine, and a primate. In particular, a subject according to the disclosure is a human.

### Antibody-Drug Conjugate Comprising an Anti-CEACAM5-Antibody

The present disclosure relates to an antibody-drug conjugate (ADC) comprising an anti-CEACAM5-antibody which is used in combination with an anti-VEGFR-2 antibody for the treatment of cancer.

The antibody-drug conjugate typically comprises an anti-CEACAM5-antibody and at least one chemotherapeutic agent. An antibody-drug conjugate (ADC) comprises an anti-CEACAM5-antibody conjugated to at least one chemotherapeutic agent. In particular, in the antibody-drug conjugate, the anti-CEACAM5-antibody is covalently attached via a cleavable or non-cleavable linker to at least one chemotherapeutic agent.

### Anti-CEACAM5-Antibody

According to an embodiment, the antibody-drug conjugate comprises a humanized anti-CEACAM5-antibody.

According to an embodiment, the antibody-drug conjugate comprises an anti-CEACAM5-antibody, wherein the anti-CEACAM5-antibody comprises a CDR-H1 consisting of SEQ ID NO: 1, CDR-H2 consisting of SEQ ID NO: 2, CDR-H3 consisting of SEQ ID NO: 3, CDR-L1 consisting of SEQ ID NO: 4, CDR-L2 consisting of amino acid sequence NTR, and CDR-L3 consisting of SEQ ID NO: 5.

In a further embodiment, the antibody-drug conjugate comprises an anti-CEACAM5-antibody, wherein the anti-CEACAM5-antibody comprises a variable domain of a heavy chain (VH) consisting of SEQ ID NO: 6 and a variable domain of a light chain (VL) consisting of SEQ ID NO: 7.

The antibody-drug conjugate comprises in a further embodiment an anti-CEACAM5-antibody, which comprises:
a variable domain of heavy chain consisting of sequence EVQLQESGPGLVKPGGSLSLSCAASGFVFSSYDMSWVRQTPERGLEWVAYISSGGGIT YAPSTVKGRFTVSRDNAKNTLYLQMNSLTSEDTAVYYCAAHYFGSSGPFAYWGQGTLV TVSS (SEQ ID NO: 6, with CDRs shown in bold characters) in which FR1-H spans amino acid positions 1 to 25, CDR1-H spans amino acid positions 26 to 33 (SEQ ID NO: 1), FR2-H spans amino acid positions 34 to 50, CDR2-H spans amino acid positions 51 to 58 (SEQ ID NO: 2), FR3-H spans amino acid positions 59 to 96, CDR3-H spans amino acid positions 97 to 109 (SEQ ID NO: 3), and FR4-H spans amino acid positions 110 to 120, and
a variable domain of light chain consisting of sequence DIQMTQSPASLSASVGDRVTITCRASENIFSYLAWYQQKPGKSPKLLVYNTRTLAEGVPS FSGSGSGTDFSLTISSLQPEDFATYYCQHHYGTPFTFGSGTKLEIK (SEQ ID NO: 7, with CDRs shown in bold characters) in which FR1-L spans amino acid positions 1 to 26, CDR1-L spans amino acid positions 27 to 32 (SEQ ID NO: 4), FR2-L spans amino acid positions 33 to 49, CDR2-L spans amino acid positions 50 to 52, FR3-L spans amino acid positions 53 to 88, CDR3-L spans amino acid positions 89 to 97 (SEQ ID NO: 5), and FR4-L spans amino acid positions 98 to 107.

In a further embodiment, the antibody-drug conjugate comprises an anti-CEACAM5-antibody, wherein the anti-CEACAM5-antibody comprises a variable domain of a heavy chain (VH) having at least 90% identity to SEQ ID NO: 6, and a variable domain of a light chain (VL) having at least 90% identity to SEQ ID NO: 7, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the antibody-drug conjugate comprises an anti-CEACAM5-antibody, wherein the anti-CEACAM5-antibody comprises a variable domain of a heavy chain (VH) having at least 92%, at least 95%, at least 98% identity to SEQ ID NO: 6, and a variable domain of a light chain (VL) having at least 92%, at least 95%, at least 98% identity to SEQ ID NO: 7, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the antibody-drug conjugate comprises an anti-CEACAM5-antibody, wherein the anti-CEACAM5-antibody comprises a heavy chain (HC) consisting of SEQ ID NO: 8 and a light chain (LC) consisting of SEQ ID NO: 9.

In a further embodiment, the antibody-drug conjugate comprises an anti-CEACAM5-antibody, wherein the anti-CEACAM5-antibody comprises a heavy chain (HC) having at least 90% sequence identity to SEQ ID NO: 8 and a light chain (LC) having at least 90% sequence identity to SEQ ID NO: 9, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the antibody-drug conjugate comprises an anti-CEACAM5-antibody, wherein the anti-CEACAM5-antibody comprises a heavy chain (HC) having at least 92%, at least 95%, at least 98% identity to SEQ ID NO: 8 and a light chain (LC) having at least 92%, at least 95%, at least 98% identity to SEQ ID NO: 9, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

The anti-CEACAM5-antibody comprised in the antibody-drug conjugate may also be a single domain antibody or a fragment thereof. In particular, a single domain antibody fragment may consist of a variable heavy chain (VHH) which comprises the CDR1-H, CDR2-H and CDR3-H of the antibodies as described above. The antibody may also be a heavy chain antibody, i.e., an antibody devoid of light chain, which may or may not contain a CH1 domain.

The single domain antibody or a fragment thereof may also comprise the framework regions of a camelid single domain antibody, and optionally the constant domain of a camelid single domain antibody.

The anti-CEACAM5-antibody comprised in the antibody-drug conjugate may also be an antibody fragment, in particular a humanized antibody fragment, selected from the group consisting of Fv, Fab, F(ab′)2, Fab′, dsFv, (dsFv)2, scFv, sc(Fv)2, and diabodies.

The antibody may also be a bispecific or multispecific antibody formed from antibody fragments, at least one antibody fragment being an antibody fragment according to the disclosure. Multispecific antibodies are polyvalent protein complexes as described for instance in EP 2 050 764 A1 or US 2005/0003403 A1.

The anti-CEACAM5-antibody and fragments thereof comprised in the antibody-drug conjugate can be produced by any technique well known in the art. In particular, said antibodies are produced by techniques as hereinafter described.

The anti-CEACAM5-antibody and fragments thereof comprised in the antibody-drug conjugate can be used in an isolated (e.g., purified) from or contained in a vector, such as a membrane or lipid vesicle (e.g., a liposome).

The anti-CEACAM5-antibody and fragments thereof comprised in the antibody-drug conjugate may be produced by any technique known in the art, such as, without limitation, any chemical, biological, genetic, or enzymatic technique, either alone or in combination.

Knowing the amino acid sequence of the desired sequence, one skilled in the art can readily produce the anti-CEACAM5-antibody and fragments thereof, by standard techniques for production of polypeptides. For instance, they can be synthesized using well-known solid phase method, in particular using a commercially available peptide synthesis apparatus (such as that made by Applied Biosystems, Foster City, California) and following the manufacturer's instructions. Alternatively, anti-CEACAM5-antibody and fragments thereof can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Anti-CEACAM5-antibody and fragments thereof are suitably separated from the culture medium by conventional immunoglobulin purification procedures such as, for example, protein A-Sepharose, hydroxyapatite chromatography, gel electrophoresis, dialysis, or affinity chromatography.

Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, the technique disclosed in the application WO2009/032661, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

The Fab of the anti-CEACAM5-antibody can be obtained by treating an antibody which specifically reacts with CEACAM5 with a protease, such as papain. Also, the Fab of the anti-CEACAM5-antibody can be produced by inserting DNA sequences encoding both chains of the Fab of the anti-CEACAM5-antibody into a vector for prokaryotic expression, or for eukaryotic expression, and introducing the vector into prokaryotic or eukaryotic cells (as appropriate) to express the Fab of the anti-CEACAM5-antibody.

The F(ab′)2 of the anti-CEACAM5-antibody can be obtained treating an antibody which specifically reacts with CEACAM5 with a protease, pepsin. Also, the F(ab′)2 of the anti-CEACAM5-antibody can be produced by binding Fab′ described below via a thioether bond or a disulfide bond.

The Fab′ of the anti-CEACAM5-antibody can be obtained treating F(ab′)2 which specifically reacts with CEACAM5 with a reducing agent, such as dithiothreitol. Also, the Fab′ of the anti-CEACAM5-antibody can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

The scFv of the anti-CEACAM5-antibody can be produced by taking sequences of the CDRs or VH and VL domains as previously described, constructing a DNA encoding an scFv fragment, inserting the DNA into a prokaryotic or eukaryotic expression vector, and then introducing the expression vector into prokaryotic or eukaryotic cells (as appropriate) to express the scFv. To generate a humanized scFv fragment, a well-known technology called CDR grafting may be used, which involves selecting the complementary determining regions (CDRs) according to the disclosure and grafting them onto a human scFv fragment framework of known three-dimensional structure (see, e. g., WO98/45322; WO 87/02671; U.S. Pat. Nos. 5,859,205; 5,585,089; 4,816,567; EP0173494).

In an embodiment, the anti-CEACAM5 antibody is tusamitamab (CAS [2349294-95-5].

### Chemotherapeutic Agents

The antibody-drug conjugate for the use according to the present disclosure typically comprises at least one chemotherapeutic agent (also referred herein to cytotoxic agent). A chemotherapeutic agent as used herein refers to an agent that kills cells, including cancer cells. Such agents favorably stop cancer cells from dividing and growing and cause tumors to shrink in size. The expression “chemotherapeutic agent” is used herein interchangeably with the expressions “cytotoxic agent”, “growth inhibitory agent” or “cytostatic drug”.

The term “chemotherapeutic agent” as used herein refers to a substance that inhibits or prevents the function of cells and/or causes destruction of cells. The term “chemotherapeutic agent” is intended to include radioisotopes, enzymes, antibiotics, and toxins such as small molecule toxins or enzymatically active toxins of bacterial, fungal, plant or animal origin, including fragments and/or variants thereof, and the various antitumor or anticancer agents disclosed below. In some embodiments, the chemotherapeutic agent is an antimetabolite.

In a further embodiment, the chemotherapeutic agent is selected from the group consisting of radioisotopes, protein toxins, small molecule toxins, and combinations thereof.

Radioisotopes include radioactive isotopes suitable for treating cancer. Such radioisotopes generally emit mainly beta-radiation. In a further embodiment, the radioisotopes are selected from the group consisting of At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, radioactive isotopes of Lu, and combinations thereof. In an embodiment, the radioactive isotope is alpha-emitter isotope, more specifically Th²²⁷, which emits alpha-radiation.

In a further embodiment, the small molecule toxins are selected from anti-metabolites, DNA-alkylating agents, DNA-cross-linking agents, DNA-intercalating agents, anti-microtubule agents, topoisomerase inhibitors, and combinations thereof.

In a further embodiment, the anti-microtubule agent is selected from the group consisting of taxanes, vinca alkaloids, maytansinoids, colchicine, podophyllotoxin, gruseofulvin, and combinations thereof.

In some embodiment a cytotoxic agent may be a maytansinoid.

According to an embodiment, maytansinoids are selected from maytansinol, maytansinol analogs, and combinations thereof.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:
(1) C-19-dechloro (U.S. Pat. No. 4,256,746) (prepared by LAH reduction of ansamytocin P2);
(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and
(3) C-20-demethoxy, C-20-acyloxy (—OCOR), +/−dechloro (U.S. Pat. No. 4,294,757) (prepared by acylation using acyl chlorides).

Specific examples of suitable analogues of maytansinol having modifications of other positions include:
(1) C-9-SH (U.S. Pat. No. 4,424,219) (prepared by the reaction of maytansinol with H2S or P2S5);
(2) C-14-alkoxymethyl (demethoxy/CH2OR) (U.S. Pat. No. 4,331,598);
(3) C-14-hydroxymethyl or acyloxymethyl (CH2OH or CH2OAc) (U.S. Pat. No. 4,450,254) (prepared from Nocardia);
(4) C-15-hydroxy/acyloxy (U.S. Pat. No. 4,364,866) (prepared by the conversion of maytansinol by Streptomyces);
(5) C-15-methoxy (U.S. Pat. Nos. 4,313,946 and 4,315,929) (isolated from Trewia nudiflora);
(6) C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

In a further embodiment, the cytotoxic conjugates of the present disclosure utilize the thiol-containing maytansinoid (DM1), formally termed N2′-deacetyl-N2′-(3-mercapto-1-oxopropyl)-maytansine, as the cytotoxic agent. DM1 is represented by the following structural formula (I):

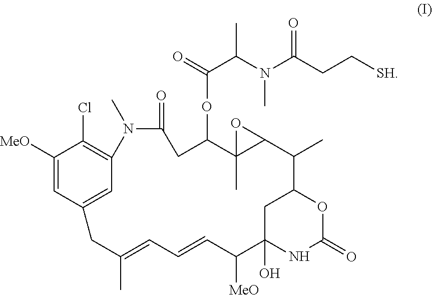 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In a further embodiment, the cytotoxic conjugates of the present disclosure utilize the thiol-containing maytansinoid DM4, formally termed N2′-deacetyl-N-2′(4-methyl-4-mercapto-1-oxopentyl)-maytansine, as the cytotoxic agent. DM4 is represented by the following structural formula (II):

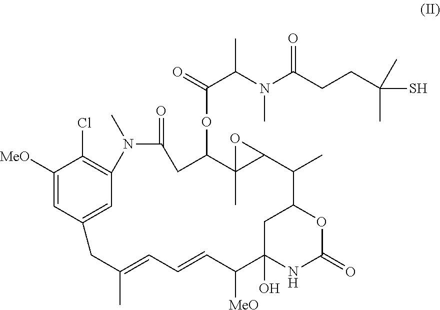 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In further embodiments of the disclosure other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 reagents and any aggregate which may be present in the solution.

Examples of these cytotoxic agents and of methods of conjugation are further given in the application WO 2008/010101 which is incorporated by reference.

The immunoconjugates according to the present disclosure can be prepared as described in the application WO 2004/091668, the entire content of which is incorporated herein by reference.

Accordingly, in a further embodiment, the maytansinoids are selected from the group consisting of N2′-deacetyl-N2′-(3-mercapto-1-oxopropyl)-maytansine (DM1) or N2′-deacetyl-N-2′(4-methyl-4-mercapto-1-oxopentyl)-maytansine (DM4), and combinations thereof.

In a further embodiment, in the antibody-drug conjugate, the anti-CEACAM5-antibody is covalently attached via a cleavable or non-cleavable linker to the at least one cytotoxic agent.

In a further embodiment, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In a further embodiment, the linker binds to a lysine or cysteine residue in the Fc region of the anti-CEACAM5 antibody. In a further embodiment, the linker forms a disulfide bond or a thioether bond with the maytansine.

In particular, the anti-CEACAM5-antibody-drug conjugate may be selected from the group consisting of:

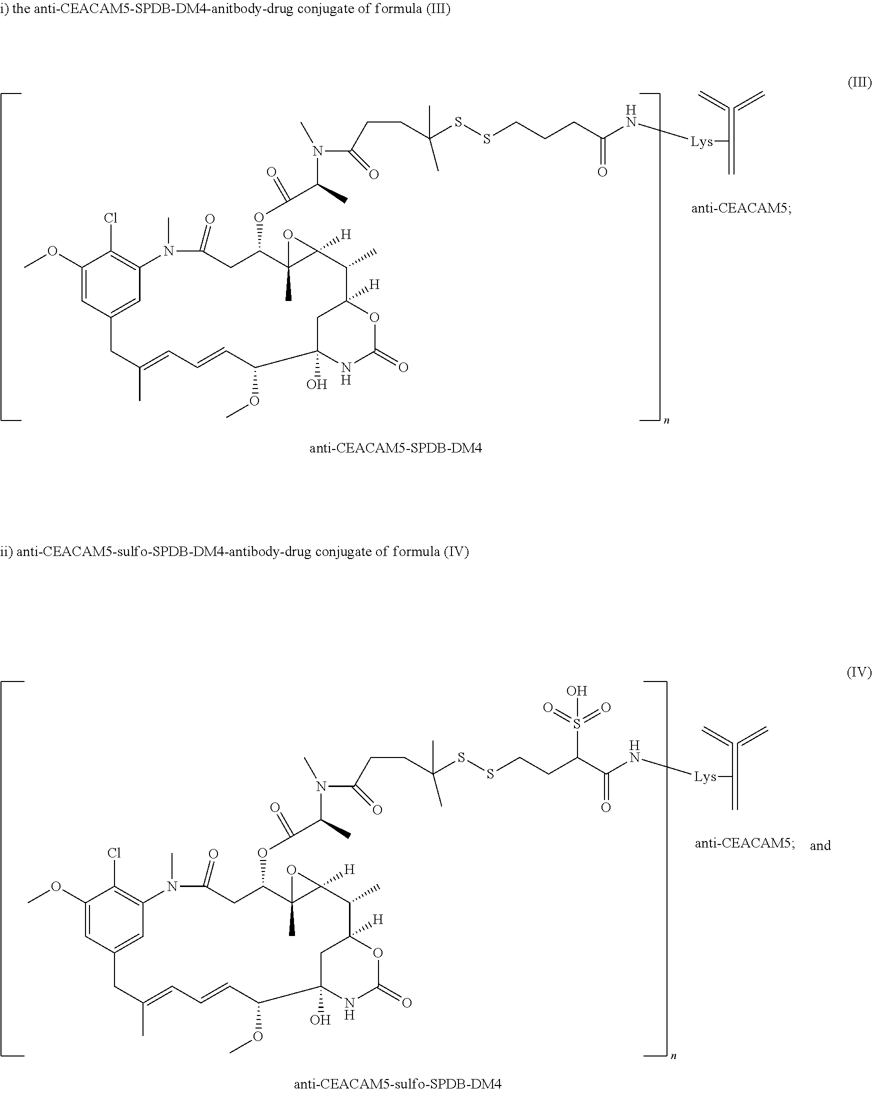

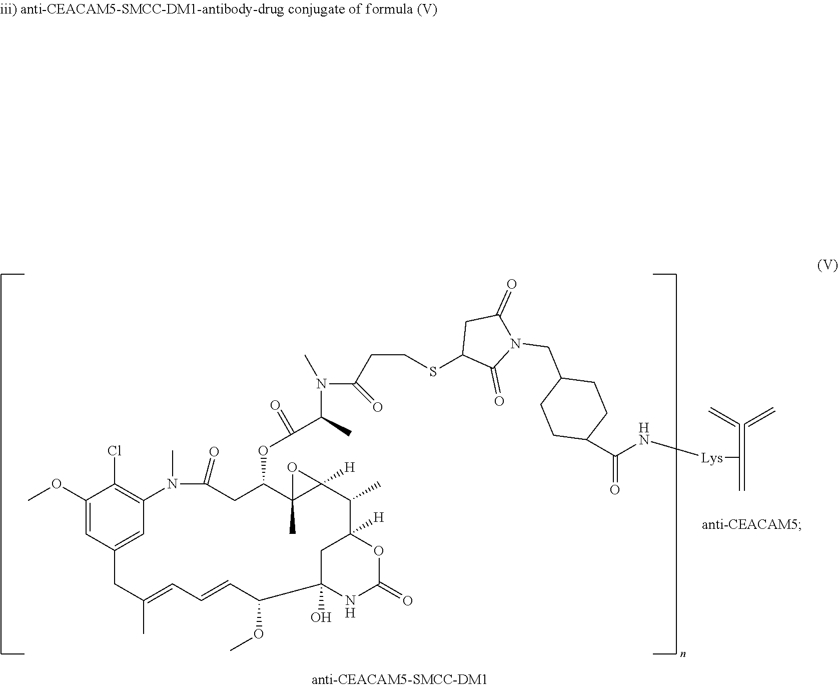

In formulas (Ill), (IV) and (V) above, “n” corresponds to the number of molecules of chemotherapeutic agent conjugated per molecule of antibody. It corresponds to the “drug-to-antibody ratio” (or “DAR”) defined below and may range from 1 to 10.

In a further embodiment, the antibody-drug conjugate of the present disclosure comprises an anti-CEACAM5-antibody. which comprises a heavy chain (VH) of SEQ ID NO: 8 and a light chain (VL) of SEQ ID NO: 9 (tusamitamab), wherein tusamitamab is covalently linked to N2′-deacetyl-N-2′(4-methyl-4-mercapto-1-oxopentyl)-maytansine (DM4) via N-succinimidyl pyridyldithiobutyrate (SPDB). Thereby, the antibody-drug conjugate tusamitamab ravtansine (huMAb2-3-SPDB-DM4) is obtained.

In an embodiment, the antibody-drug conjugate of the present disclosure is tusamitamab ravtansine (CAS [2254086-60-5]).

“Linker”, as used herein, means a chemical moiety comprising a covalent bond or a chain of atoms that covalently attaches the antibody to the chemotherapeutic agent moiety (e.g., a cytostatic agent, a cytotoxic agent or a growth inhibitory agent). Suitable linkers are well known in the art and include disulfide groups, thioether groups, acid labile groups, photolabile groups, peptidase labile groups and esterase labile groups.

The conjugates may be prepared by in vitro methods. In order to link a drug or prodrug to the antibody, e.g., a chemotherapeutic agent, a linking group is used. Suitable linking groups are well known in the art and include disulfide groups, thioether groups, acid labile groups, photolabile groups, peptidase labile groups and esterase labile groups. Conjugation of an antibody with a chemotherapeutic agent of the disclosure, such as a cytotoxic agent, may be made using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the chemotherapeutic agent in the cell. For example, an acid-labile linker, a peptidase-sensitive linker, an esterase labile linker, a photolabile linker or a disulfide-containing linker (See e.g., U.S. Pat. No. 5,208,020) may be used. The linker may be also a “non-cleavable linker” (for example SMCC linker) that might led to better tolerance in some cases.

In general, the conjugate can be obtained by a process comprising the steps of:
(i) bringing into contact an optionally-buffered aqueous solution of a cell-binding agent (e.g., an antibody according to the disclosure) with solutions of a linker and a chemotherapeutic agent, such as a cytotoxic compound (or agent);
(ii) then optionally separating the conjugate which was formed in (i) from the unreacted cell-binding agent (e.g., antibody of the disclosure) and unreacted chemotherapeutic agent, such as unreacted cytotoxic compound (or agent).

The aqueous solution of cell-binding agent can be buffered with buffers such as, e.g., potassium phosphate, acetate, citrate or N-2-Hydroxyethylpiperazine-N′-2-ethanesulfonic acid (Hepes buffer). The buffer depends upon the nature of the cell-binding agent (e.g., antibody of the disclosure). The chemotherapeutic agent, such as the cytotoxic compound (or agent), is in solution in an organic polar solvent, e.g., dimethyl sulfoxide (DMSO) or dimethylacetamide (DMA).

The reaction temperature is usually comprised between 20° C. and 40° C. The reaction time can vary from 1 hour to 24 hours. The reaction between the cell-binding agent and the chemotherapeutic agent, such as the cytotoxic agent, can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified, for example from aggregates, e.g., by SEC, adsorption chromatography (such as ion exchange chromatography, IEC), hydrophobic interaction chromatography (HIC), affinity chromatography, mixed-support chromatography such as hydroxyapatite chromatography, or high-performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent (e.g., antibody of the disclosure) in the solution, the pH of the solution, high shearing forces, the number of bonded dimers and their hydrophobic character, the temperature (see Wang & Gosh, 2008, J. Membrane Sci., 318: 311-316, and references cited therein); note that the relative influence of some of these parameters is not clearly established. In the case of proteins and antibodies, the person skilled in the art will refer to Cromwell et al. (2006, AAPS Journal, 8(3): E572-E579). The content in aggregates can be determined with techniques well known to the skilled person, such as SEC (see Walter et al., 1993, Anal. Biochem., 212(2): 469-480).

After step (i) or (ii), the conjugate-containing solution can be submitted to an additional step (iii) of chromatography, ultrafiltration and/or diafiltration.

The conjugate is recovered at the end of these steps in an aqueous solution.

In a further embodiment, the antibody-drug conjugate according to the disclosure is characterized by a “drug-to-antibody ratio” (or “DAR”) ranging from 1 to 10, or from 2 to 5, or from 3 to 4. This is generally the case of conjugates including maytansinoid molecules.

This DAR number can vary with the nature of the antibody and of the drug (i.e. the chemotherapeutic agent, such as a cytotoxic agent or a growth-inhibitory agent) used along with the experimental conditions used for the conjugation (like the ratio chemotherapeutic agent (e.g., growth-inhibitory agent)/antibody, the reaction time, the nature of the solvent and of the cosolvent if any). Thus the contact between the antibody and the chemotherapeutic agent, such as a cytotoxic agent or a growth-inhibitory agent, leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λD and 280 nm. 280 nm is a wavelength generally used for measuring protein concentration, such as antibody concentration. The wavelength λD is selected so as to allow discriminating the drug from the antibody, i.e., as readily known to the skilled person, λD is a wavelength at which the drug (i.e., chemotherapeutic agent) has a high absorbance and λD is sufficiently remote from 280 nm to avoid substantial overlap in the absorbance peaks of the drug and antibody. AD may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λD (AλD) and at 280 nm (A280) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

$$A λ D = ( cD \times ε D λ D ) + ( cA \times ε A λ D )$$

$$A 280 = ( cD \times ε D 280 ) + ( cA \times ε A 280 )$$

wherein:
cD and cA are respectively the concentrations in the solution of the drug (i.e., chemotherapeutic agent) and of the antibody
εDλD and εD280 are respectively the molar extinction coefficients of the drug at λD and 280 nm
εAλD and εA280 are respectively the molar extinction coefficients of the antibody at λD and 280 nm.

Resolution of these two equations with two unknowns leads to the following equations:

$$c D = [ ( ε A 280 \times A λ D ) - ( ε A λ D \times A 280 ) ] \text{} / [ ( ε D λ D \times ε A 280 ) - ( ε A λ D \times ε D 280 ) ]$$

$$cA = [ A 280 - ( cD \times ε D 280 ) ] / ε A 280$$

The average DAR is then calculated from the ratio of the drug concentration to that of the antibody: DAR=cD/cA.

### Anti-VEGFR-2 Antibody

The antibody-drug conjugate (ADC) comprising an anti-CEACAM5-antibody is to be used in combination with an anti-VEGFR-2 antibody for the treatment of cancer.

In one embodiment, the anti-VEGFR-2 antibody is a monoclonal antibody, or a fragment thereof having antagonist activity to VEGFR-2. In one embodiment, the anti-VEGFR-2 antibody is an IgG.

The anti-VEGFR-2 antibody is preferably adapted to the patient. For example, an anti-mouse VEGFR-2 antibody such as DC-101 is preferably used on mice, and an anti-human VEGFR-2 antibody on humans.

In one embodiment, the anti-VEGFR-2 antibody is ramucirumab (CAS number 947687-13-0). It is a fully human monoclonal IgG1 antibody against human VEGFR-2.

In one embodiment, the anti-VEGFR-2 antibody comprises the light chain and heavy chain CDRs of ramucirumab.

In one embodiment, the anti-VEGFR-2 antibody comprises the variable domain of heavy chain (VH) and the variable domain of light chain (VL) of -ramucirumab.

In a further embodiment, the anti-VEGFR-2 antibody comprises a heavy chain (HC) having at least 92%, at least 95%, at least 98% identity to SEQ ID NO: 10 and a light chain (LC) having at least 92%, at least 95%, at least 98% identity to SEQ ID NO: 11.

The anti-VEGFR-2 antibody comprised may also be a single domain antibody or a fragment thereof. In particular, a single domain antibody fragment may consist of a variable heavy chain (VHH) which comprises the CDR1-H, CDR2-H and CDR3-H of the antibodies as described above. The antibody may also be a heavy chain antibody, i.e., an antibody devoid of light chain, which may or may not contain a CH1 domain.

The single domain antibody or a fragment thereof may also comprise the framework regions of a camelid single domain antibody, and optionally the constant domain of a camelid single domain antibody.

The anti-VEGFR-2 antibody may also be an antibody fragment, in particular a humanized antibody fragment, selected from the group consisting of Fv, Fab, F(ab′)2, Fab′, dsFv, (dsFv)2, scFv, sc(Fv)2, and diabodies.

The antibody may also be a bispecific or multispecific antibody formed from antibody fragments, at least one antibody fragment being an antibody fragment according to the disclosure. The anti-VEGFR-2 antibody and fragments thereof can be produced by any technique well known in the art. In particular, said antibodies are produced by techniques as already described.

The anti-VEGFR-2 antibody and fragments thereof can be used in an isolated (e.g., purified) from or contained in a vector, such as a membrane or lipid vesicle (e.g., a liposome).

The anti-VEGFR-2 antibody and fragments thereof may be produced by any technique known in the art, such as, without limitation, any chemical, biological, genetic, or enzymatic technique, either alone or in combination.

### Combined Treatment

According to the present disclosure, the antibody-drug conjugate comprising an anti-CEACAM5-antibody is for use for treating cancer in combination with an anti-VEGFR-2 antibody. The disclosure also relates to an anti-VEGFR-2 antibody for use for treating cancer in combination with the antibody-drug conjugate comprising an anti-CEACAM5-antibody.

As used herein, the expression “in combination with” means that the anti-VEGFR-2 antibody is administered before, after, or concurrent with the antibody-drug conjugate comprising an anti-CEACAM5-antibody. In some embodiments, the term “in combination with” includes sequential or concomitant administration of the antibody-drug conjugate comprising an anti-CEACAM5-antibody and the anti-VEGFR-2 antibody. Methods to treat cancer (GC, GEJ cancer, NSQ NSCLC) includes administering an antibody-drug conjugate comprising an anti-CEACAM5-antibody (e.g., tusamitamab ravtansine) in combination with the anti-VEGFR-2 antibody for additive or synergistic activity.

For example, when administered “before” the antibody-drug conjugate comprising an anti-CEACAM5-antibody, the anti-VEGFR-2 antibody may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the antibody-drug conjugate comprising an anti-CEACAM5-antibody. When administered “after” the antibody-drug conjugate comprising an anti-CEACAM5-antibody, the anti-VEGFR-2 antibody may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the antibody-drug conjugate comprising an anti-CEACAM5-antibody. “Concurrent” administration comprising the ADC means that the anti-VEGFR-2 antibody is administered to the patient in a separate dosage form within less than 5 minutes (before, after, or at the same time) of administration of the antibody-drug conjugate comprising an anti-CEACAM5-antibody, or administered to the patient as a single combined dosage formulation comprising both the anti-VEGFR-2 antibody and the antibody-drug conjugate comprising an anti-CEACAM5-antibody or as separate formulations, one comprising the antibody-drug conjugate comprising an anti-CEACAM5-antibody and the other comprising the anti-VEGFR-2 antibody.

In some embodiments, the method of treating cancer is administering to a patient in need thereof an effective amount of an antibody-drug conjugate comprising an anti-CEACAM5-antibody before administering the anti-VEGFR-2 antibody.

In some embodiments, the method of treating cancer is administering to a patient in need thereof an effective amount of an antibody-drug conjugate comprising an anti-CEACAM5-antibody comprising an anti-CEACAM5 antibody after administering the anti-VEGFR-2 antibody.

The present disclosure also relates to a method of treatment of cancer in a patient in need thereof, comprising administering the antibody-drug conjugate comprising an anti-CEACAM5-antibody, and administering an anti-VEGFR-2 antibody to a patient in need thereof.

The present disclosure also relates to a combination comprising an anti-VEGFR-2 antibody and an antibody-drug conjugate comprising an anti-CEACAM5-antibody for use for treating cancer.

A method of treatment or a use, as disclosed herein, may achieve a synergistic effect in reducing tumor size.

A method of treatment or a use, as disclosed herein, may achieve a synergistic effect in inhibiting tumor growth.

The present disclosure also relates to a combination for the manufacture of a medicament for the treatment of cancer, comprising an anti-VEGFR-2 antibody and an antibody-drug conjugate comprising an anti-CEACAM5-antibody.

In an embodiment, said combination permits a simultaneous, separate or a sequential administration of the anti-VEGFR-2 antibody and the antibody-drug conjugate comprising an anti-CEACAM5-antibody.

In an embodiment, said combination permits a simultaneous administration of the anti-VEGFR-2 antibody and the antibody-drug conjugate comprising an anti-CEACAM5-antibody.

In an embodiment, said combination permits a separate administration of the anti-VEGFR-2 antibody and the antibody-drug conjugate comprising an anti-CEACAM5-antibody.

In an embodiment, said combination permits a sequential administration of the anti-VEGFR-2 antibody and the antibody-drug conjugate comprising an anti-CEACAM5-antibody.

In a further embodiment, combinations according to the disclosure are pharmaceutical combinations.

The disclosure also relates to the antibody-drug conjugate comprising an anti-CEACAM5-antibody for use for treating cancer in a patient in need thereof who receives, simultaneously, separately, or sequentially an anti-VEGFR-2 antibody.

In an embodiment, the cancer is a carcinoma, a sarcoma or a blastoma. In a further embodiment, the cancer is a carcinoma.

According to an embodiment, the cancer is a cancer expressing CEACAM5.

According to an embodiment, the cancer is selected from hepatocellular carcinoma, colorectal cancer, gastric cancer, gastroesophageal junction (GEJ) adenocarcinoma, lung cancer (e.g., non-squamous non-small cell lung cancer), uterus cervix cancer, pancreas cancer, ovary cancer, thyroid cancer, bladder cancer, endometrium cancer, breast cancer, liver cancer (for instance cholangiocarcinoma), prostate cancer or skin cancer.

According to an embodiment, the cancer is gastric cancer or gastroesophageal junction adenocarcinoma (GEJ).

According to an embodiment, the cancer is a gastric cancer.

According to an embodiment, the cancer is a lung cancer. A lung cancer may be a non-squamous non-small-cell lung cancer (NSQ NSCLC).

According to an embodiment, the patient is a patient with malignant tumor, in particular with a malignant solid tumor, and more specifically with locally advanced or metastatic solid malignant tumor. A metastatic solid malignant tumor may be a metastatic cancer, for example a metastatic carcinoma. A cancer or a carcinoma may be as above indicated.

In some embodiments, the cancer is a CEACAM5-positive cancer. A CEACAM5-positive cancer is defined as cancer for which a CEACAM5 immunohistochemical [IHC] intensity is ≥2+ in ≥50% of cancer cells or ≥2+ intensity in ≥1% and <50% of the cells tumors (or cancer cells).

In certain embodiments, the patient has a cancer having a negative or low CEACAM5 expression on tumor cells. A negative or low CEACAM5 expression on tumor cells defined as being ≥2+ intensity in <1% of cells, as measured by immunohistochemistry (IHC).

In certain embodiments, the patient has a cancer having a moderate CEACAM5 expression on tumor cells. A moderate CEACAM5 expression on tumor cells may be defined as being ≥2+ intensity in ≥1% and <50% of cancer cells, as measured by immunohistochemistry.

In certain embodiments, the patient has a cancer having a high CEACAM5 expression on tumor cells. A high CEACAM5 expression on tumor cells may be defined as being ≥2+ intensity in ≥50% of cancer cells, as measured by immunohistochemistry.

In some embodiments, the patient has a cancer having a CEACAM5 expression defined as a CEACAM5 immunohistochemistry (IHC) intensity of at least about 2+ in at least about 50% of tumor cells.

According to some embodiments, the antibody-drug conjugate comprising an anti-CEACAM5-antibody and an anti-VEGFR-2 antibody are administered simultaneously, separately, or sequentially to a patient in need thereof.

According to an embodiment, the antibody-drug conjugate comprising an anti-CEACAM5-antibody and an anti-VEGFR-2 antibody are simultaneously administered to a patient in need thereof. For example, antibody-drug conjugate comprising an anti-CEACAM5-antibody and an anti-VEGFR2 antibody are administered on day one of a cycle, approximatively at the same time. A simultaneous administration of the antibody-drug conjugate comprising an anti-CEACAM5-antibody and an anti-VEGFR-2 antibody may be by the same route.

According to an embodiment, the antibody-drug conjugate comprising an anti-CEACAM5-antibody and an anti-VEGFR-2 antibody are separately administered to a patient in need thereof. For example, antibody-drug conjugate comprising an anti-CEACAM5-antibody and an anti-VEGFR2 antibody are administered on day one of a cycle, by separate routes or at separates location of the body of said patient. A separate administration of the antibody-drug conjugate comprising an anti-CEACAM5-antibody and an anti-VEGFR-2 antibody may be at the same time or at close times, e.g., 5 min or less.

According to an embodiment, the antibody-drug conjugate comprising an anti-CEACAM5-antibody and an anti-VEGFR-2 antibody are sequentially administered to a patient in need thereof. For example, antibody-drug conjugate comprising an anti-CEACAM5-antibody and an anti-VEGFR-2 antibody are administered on day one of a cycle, at different times, for example the anti-CEACAM5-antibody is administered one to three hours after the anti-VEGFR-2 antibody. A sequential administration of the antibody-drug conjugate comprising an anti-CEACAM5-antibody and an anti-VEGFR-2 antibody may be by separate routes or by a same route. A sequential administration of the antibody-drug conjugate comprising an anti-CEACAM5-antibody and an anti-VEGFR-2 antibody may comprise administration of the anti-CEACAM5-antibody after the anti-VEGFR-2 antibody. The anti-CEACAM5-antibody may be administered about 0.5 hr, 1 hr, 2 hrs, 3 hrs, 4 hrs, 5 hrs or about 6 hrs after the anti-VEGFR-2 antibody. The anti-CEACAM5-antibody may be administered about 1 hr after the anti-VEGFR-2 antibody.

In a further embodiment, the antibody-drug conjugate comprising an anti-CEACAM5-antibody and an anti-VEGFR-2 antibody are formulated (i) in a single pharmaceutical composition comprising the antibody-drug conjugate and the anti-VEGFR-2 antibody, or (ii) in the form of two separate pharmaceutical compositions, wherein one pharmaceutical composition comprises the antibody-drug conjugate comprising an anti-CEACAM5-antibody, and the other pharmaceutical composition comprises the anti-VEGFR-2 antibody.

In a further embodiment, the antibody-drug conjugate comprising an anti-CEACAM5-antibody and an anti-VEGFR-2 antibody are formulated (i) in a single pharmaceutical composition comprising the antibody-drug conjugate and the anti-VEGFR-2 antibody, and at least one pharmaceutically acceptable excipient, or (ii) in the form of two separate pharmaceutical compositions, wherein one pharmaceutical composition comprises the antibody-drug conjugate comprising an anti-CEACAM5-antibody and at least one pharmaceutically acceptable excipient, and the other pharmaceutical composition comprises the anti-VEGFR-2 antibody and at least one pharmaceutically acceptable excipient.

In the case of formulation of the antibody-drug conjugate and the anti-VEGFR-2 antibody in two separate pharmaceutical compositions, the two separate pharmaceutical compositions may be administered simultaneously, separately, or sequentially, to a patient in need thereof. In some embodiments, the two separate pharmaceutical compositions may be sequentially administered to a patient in need thereof.

In a sequential administration, the period of time between the administration of the anti-VEGFR-2 antibody and the antibody-drug conjugate comprising an anti-CEACAM5-antibody may last from about a few minutes to about several hours, days, or weeks. In some embodiments, the period of time may range from about 5 minutes to about 3 hours, for example from 10 minutes to about 2.5 hours, from about 30 minutes to about 2 hours, or from about 1 hour to about 1.5 hours. A period of time between may last about 5 minutes, about 10 minutes, about 30 minutes, about 1 hour, 1.5 hours, about 2 hours, about 2.5 hours or about 3 hours.

In some embodiments, the anti-VEGFR-2 is administered over one hour.

In some embodiments, the antibody-drug conjugate comprising an anti-CEACAM5-antibody is administered over 1.5 hours.

In some embodiments, in a sequential administration on a same day of a cycle, the period of time between the administration of the anti-VEGFR-2 antibody and the antibody-drug conjugate comprising an anti-CEACAM5-antibody may range from about 5 minutes to about 3 hours, for example from 10 minutes to about 2.5 hours, from about 30 minutes to about 2 hours, or from about 1 hour to about 1.5 hours. In a sequential administration on a same day of a cycle, a period of time between may last about 5 minutes, about 10 minutes, about 30 minutes, about 1 hour, 1.5 hours, about 2 hours, about 2.5 hours or about 3 hours.

In an embodiment, in a sequential administration on a same day of a cycle, the period of time between the administration of the anti-VEGFR-2 antibody and the antibody-drug conjugate comprising an anti-CEACAM5-antibody may be at least one hour.

In a sequential administration, the antibody-drug conjugate (ADC) comprising an anti-CEACAM5-antibody may be administered after or before the anti-VEGFR-2 antibody.

In some embodiments, the antibody-drug conjugate comprising an anti-CEACAM5-antibody may be administered before the anti-VEGFR-2 antibody.

In some embodiments, the antibody-drug conjugate comprising an anti-CEACAM5-antibody may be administered after the anti-VEGFR-2 antibody.

In some embodiments, the sequence of administration of the antibody-drug conjugate comprising an anti-CEACAM5-antibody and of the anti-VEGFR-2 antibody may be the same for all cycles of treatment.

In some embodiments, the sequence of administration of the antibody-drug conjugate comprising an anti-CEACAM5-antibody and of the anti-VEGFR-2 antibody may vary along the cycles of treatment. In some embodiments, one or more cycles of a treatment may comprise a first sequence of administration and one or more cycles of said treatment may comprise a second sequence of administration, the first and second sequences being different.

In some embodiments, for example in a use for treating a cancer, in a first cycle of treatment, the antibody-drug conjugate comprising an anti-CEACAM5-antibody may be administered after the anti-VEGFR-2 antibody, and in a subsequent additional cycle of treatment the antibody-drug conjugate comprising an anti-CEACAM5-antibody may be administered after the anti-VEGFR-2 antibody.

In some embodiments, for example in a use for treating a cancer, in a first cycle of treatment, the antibody-drug conjugate comprising an anti-CEACAM5-antibody may be administered after the anti-VEGFR-2 antibody, and in a subsequent additional cycle of treatment the antibody-drug conjugate comprising an anti-CEACAM5-antibody may be administered before the anti-VEGFR-2 antibody.

In some embodiments, for example in a use for treating a cancer, in a first cycle of treatment, the antibody-drug conjugate comprising an anti-CEACAM5-antibody may be administered before the anti-VEGFR-2 antibody, and in a subsequent additional cycle of treatment the antibody-drug conjugate comprising an anti-CEACAM5-antibody may be administered before the anti-VEGFR-2 antibody.

In some embodiments, for example in a use for treating a cancer, in a first cycle of treatment, the antibody-drug conjugate comprising an anti-CEACAM5-antibody may be administered before the anti-VEGFR-2 antibody, and in a subsequent additional cycle of treatment the antibody-drug conjugate comprising an anti-CEACAM5-antibody may be administered after the anti-VEGFR-2 antibody.

In some embodiments, for example in a use for treating a cancer such as gastric cancer, GEJ cancer or lung cancer, the antibody-drug conjugate comprising an anti-CEACAM5-antibody may be administered after the anti-VEGFR-2 antibody for all additional cycles of treatment.

A treatment, or course of treatment, may comprise at least one cycle of treatment.

In some embodiments, a treatment may comprise a first cycle of treatment, i.e., cycle 1, and at least one additional cycle of treatment, i.e., cycle(s) 2, 3, 4, 5, 6, 7, 8, 9, 10, 11, 12, 13, 14, 15, 16, or more.

The first cycle and the additional cycle(s) may be identical or different.

For example, the first cycle may comprise an administration of a loading dose (or first dose), and the additional cycle(s) may comprise an administration of a subsequent dose (or second), i.e., different dosages for the loading and subsequent doses.

Alternatively, the first and additional cycles may comprise an administration of a same dose, i.e., same dosage for the loading and subsequent doses.

In some embodiments, the antibody-drug conjugate comprising an anti-CEACAM5-antibody may be administered at a loading dose in a first cycle and at a subsequent dose in additional cycle(s), i.e., different dosages for the loading and subsequent doses.

In some embodiments, the antibody-drug conjugate comprising an anti-CEACAM5-antibody may be administered at a same dose in a first cycle and additional cycle(s), i.e., same dosage for the loading and subsequent doses.

In some embodiments, the anti-VEGFR-2 antibody may be administered at a loading dose in a first cycle and at a subsequent dose in additional cycle(s), i.e., different dosages for the loading and subsequent doses.

In some embodiments, the anti-VEGFR-2 antibody may be administered at a same dose in a first cycle and additional cycle(s), i.e., same dosage for the loading and subsequent doses.

In some embodiments, the antibody-drug conjugate comprising an anti-CEACAM5-antibody may be administered at a loading dose in a first cycle and at a subsequent dose in additional cycle(s), i.e., different dosages for the loading and subsequent doses, and the anti-VEGFR-2 antibody may be administered at a same dose in a first cycle and additional cycle(s), i.e., same dosage for the loading and subsequent doses.

In some embodiments, the antibody-drug conjugate comprising an anti-CEACAM5-antibody may be administered at a same dose in a first cycle and additional cycle(s) and the anti-VEGFR-2 antibody may be administered at a same dose in a first cycle and additional cycle(s), i.e., same dosage for the loading and subsequent doses.

A cycle of treatment may last from about 1 to about 6 weeks, from 1 to 4 weeks, from 1 to 3 weeks.

In some embodiments, a cycle of treatment may last at least about two weeks.

In some embodiments, a cycle of treatment may last at least about three weeks.

In some embodiment, a cycle of treatment may comprise a period of treatment at least on day 1, for example on day 1, 2, 3, 4, 5 or 6, of the cycle and a period of rest lasting until the completion of said cycle. The periods of treatment and the periods of rest may be identical or different between a first cycle and an at least one additional cycle. In some embodiments, the periods of treatment and the periods of rest may be identical between a first cycle and an at least one additional cycle.

In some embodiment, a cycle of treatment, i.e., first and additional cycles, may comprise a period of treatment on day 1 of the cycle and a period of rest lasting until the completion of said cycle.

In some embodiments, the antibody-drug conjugate comprising an anti-CEACAM5-antibody and the anti-VEGFR-2 antibody may be administered at day 1 of a first cycle of treatment and at day 1 of an at least one additional cycle(s) of treatment.

The antibody-drug conjugate comprising an anti-CEACAM5-antibody and the anti-VEGFR2 antibody may be administered at day 1 of each cycle of treatment.

A treatment (or course of treatment) may comprise at least a first cycle (cycle 1) of treatment and at least one additional (subsequent) cycle. A treatment may comprise from 2 to 16, from 3 to 15, from 4 to 14, from 5 to 13, from 6 to 12, from 7 to 11, from 8 to 10, or about 9 cycles. A treatment may comprise 2, 3, 4, 5, 6, 7, 8, 9, 10, 11, 12, 13, 14, 15, 16 or more cycles.

In some embodiments, it is disclosed an antibody-drug conjugate comprising an anti-CEACAM5-antibody and a cytotoxic agent for use for treating a cancer in combination with an anti-VEGFR-2 antibody, wherein the antibody-drug conjugate is administered at a dose of 60 mg/m² to 210 mg/m², or from about 80 to about 170 mg/m², or from about 100 to about 170 mg/m², or from about 120 to about 170 mg/m², or from about 135 to about 170 mg/m², or from about 150 to about 170 mg/m².

The anti-VEGFR-2 antibody is administered at a dose of 2 mg/kg to 20 mg/kg, or from about 4 to about 15 mg/kg, or from about 6 to about 10 mg/kg, or at about 8 mg/kg, or at about 10 mg/kg.

In some embodiments, the antibody-drug conjugate is administered at a dose of from about 60 to about 210 mg/m², or from about 80 to about 170 mg/m², or from about 100 to about 150 mg/m².

In various embodiments, the antibody-drug conjugate comprising the anti-CEACAM5 antibody is administered at a dose of about 60, 70, 80, 90, 100, 110, 120, 130, 135, 140, 145, 150, 155, 160, 165, 170, 175, 180, 185, 190, 195, 200, 205, or about 210 mg/m².

In various embodiments, the antibody-drug conjugate comprising the anti-CEACAM5 antibody is administered at a dose of about 60, 80, 100, 120, 135, 150, 170, 180, 190 or about 210 mg/m².

According to an embodiment, the antibody-drug conjugate comprising the anti-CEACAM5 antibody is administered at a dose of about 80 mg/m².

According to an embodiment, the antibody-drug conjugate comprising the anti-CEACAM5 antibody is administered at a dose of about 100 mg/m².

According to an embodiment, the antibody-drug conjugate comprising the anti-CEACAM5 antibody is administered at a dose of about 120 mg/m².

According to an embodiment, the antibody-drug conjugate comprising the anti-CEACAM5 antibody is administered at a dose of about 135 mg/m².

According to an embodiment, the antibody-drug conjugate comprising the anti-CEACAM5 antibody is administered at a dose of about 150 mg/m².

According to an embodiment, the antibody-drug conjugate comprising the anti-CEACAM5 antibody is administered at a dose of about 170 mg/m².

According to an embodiment, the antibody-drug conjugate comprising the anti-CEACAM5 antibody is administered at a dose of about 80, 100, 120, 135, 150 or about 170 mg/m², as a loading dose (or first dose).

According to an embodiment, the antibody-drug conjugate comprising the anti-CEACAM5 antibody is administered at a dose of about 120, 135, 150 or about 170 mg/m², as a loading dose.

According to an embodiment, the antibody-drug conjugate comprising the anti-CEACAM5 antibody is administered at a dose of about 120, 150, or about 170 mg/m², as a loading dose.

According to an embodiment, the antibody-drug conjugate comprising the anti-CEACAM5 antibody is administered at a dose of about 80 mg/m², as a loading dose.

According to an embodiment, the antibody-drug conjugate comprising the anti-CEACAM5 antibody is administered at a dose of about 100 mg/m², as a loading dose.

According to an embodiment, the antibody-drug conjugate comprising the anti-CEACAM5 antibody is administered at a dose of about 120 mg/m², as a loading dose.

According to an embodiment, the antibody-drug conjugate comprising the anti-CEACAM5 antibody is administered at a dose of about 135 mg/m², as a loading dose.

According to an embodiment, the antibody-drug conjugate comprising the anti-CEACAM5 antibody is administered at a dose of about 150 mg/m², as a loading dose.

According to an embodiment, the antibody-drug conjugate comprising the anti-CEACAM5 antibody is administered at a dose of about 170 mg/m², as a loading dose.

According to an embodiment, the loading dose is for a cycle of treatment of 2 weeks.

According to an embodiment, the loading dose is for a cycle of treatment of 3 weeks.

A loading dose may be administered at day 1 of the first cycle.

On day 1 of a first cycle, the antibody-drug conjugate comprising an anti-CEACAM5-antibody may be administered at a dose of about 80 mg/m², 100 mg/m², 120 mg/m², 135 mg/m², 150 mg/m² or about 170 mg/m².

On day 1 of a first cycle, the antibody-drug conjugate comprising an anti-CEACAM5-antibody may be administered at a dose of about 120 mg/m², 150 mg/m² or about 170 mg/m².

On day 1 of a first cycle, the antibody-drug conjugate comprising an anti-CEACAM5-antibody may be administered at a dose of about 80 mg/m² or about 100 mg/m².

According to an embodiment, the antibody-drug conjugate comprising the anti-CEACAM5 antibody may be administered at a dose of about 80, 100, 120, 135, 150 or about 170 mg/m², as a subsequent dose (or second dose).

According to an embodiment, the antibody-drug conjugate comprising the anti-CEACAM5 antibody may be administered at a dose of about 80 mg/m², as a subsequent dose.

According to an embodiment, the antibody-drug conjugate comprising the anti-CEACAM5 antibody may be administered at a dose of about 100 mg/m², as a subsequent dose.

According to an embodiment, the antibody-drug conjugate comprising the anti-CEACAM5 antibody may be administered at a dose of about 120 mg/m², as a subsequent dose.

According to an embodiment, the antibody-drug conjugate comprising the anti-CEACAM5 antibody may be administered at a dose of about 135 mg/m², as a subsequent dose.

According to an embodiment, the antibody-drug conjugate comprising the anti-CEACAM5 antibody may be administered at a dose of about 150 mg/m², as a subsequent dose.

According to an embodiment, the antibody-drug conjugate comprising the anti-CEACAM5 antibody may be administered at a dose of about 170 mg/m², as a subsequent dose.

A subsequent dose may be administered at day 1 of cycle(s) subsequent to the first cycle (the subsequent or additional cycles).

On day 1 of additional cycles (at least one cycle subsequent to the first cycle), the antibody-drug conjugate comprising an anti-CEACAM5-antibody may be administered at a dose of about 80 mg/m².

On day 1 of additional cycles (at least one cycle subsequent to the first cycle), the antibody-drug conjugate comprising an anti-CEACAM5-antibody may be administered at a dose of about 100 mg/m².

On day 1 of additional cycles (at least one cycle subsequent to the first cycle), the antibody-drug conjugate comprising an anti-CEACAM5-antibody may be administered at a dose of about 120 mg/m².

On day 1 of additional cycles (at least one cycle subsequent to the first cycle), the antibody-drug conjugate comprising an anti-CEACAM5-antibody may be administered at a dose of about 135 mg/m².

On day 1 of additional cycles (at least one cycle subsequent to the first cycle), the antibody-drug conjugate comprising an anti-CEACAM5-antibody may be administered at a dose of about 150 mg/m².

On day 1 of additional cycles (at least one cycle subsequent to the first cycle), the antibody-drug conjugate comprising an anti-CEACAM5-antibody may be administered at a dose of about 170 mg/m².

According to an embodiment, the antibody-drug conjugate comprising the anti-CEACAM5 antibody is administered at a dose of about 80, 100, 120, 135, 150 or about 170 mg/m², as a loading dose, on a first cycle of treatment, e.g., at day 1, and then at a dose of about 80, 100, 120, 135, 150 or about 170 mg/m², as a subsequent dose, e.g., at day 1, on additional cycle(s).

According to an embodiment, the antibody-drug conjugate comprising the anti-CEACAM5 antibody is administered at a dose of about 100, 120, 135, 150 or about 170 mg/m², as a loading dose, on a first cycle of treatment, e.g., at day 1, and then at a dose of about 80 or about 100 mg/m², as a subsequent dose, e.g., at day 1, on additional cycle(s).

The antibody-drug conjugate comprising an anti-CEACAM5-antibody may be administered at a dose of about 80 mg/m², 100 mg/m², 120 mg/m², 135 mg/m², 150 mg/m² or about 170 mg/m² on day 1 of a first cycle of treatment and at a dose of about 80 mg/m² or about 100 mg/m² on day 1 of additional cycles.

At day 1 of a first cycle of treatment the antibody-drug conjugate may be administered at a dose of about 80 mg/m², 100 mg/m², 120 mg/m², 135 mg/m², 150 mg/m² or about 170 mg/m², and the cycle of treatment may be 2 weeks.

According to an embodiment, the antibody-drug conjugate comprising the anti-CEACAM5 antibody may be administered at a dose of about 80 mg/m², at day 1 on a first cycle of treatment, and the cycle may be about 2 weeks.

According to an embodiment, the antibody-drug conjugate comprising the anti-CEACAM5 antibody may be administered at a dose of about 100 mg/m², at day 1 on a first cycle of treatment, and the cycle may be about 2 weeks.

According to an embodiment, the antibody-drug conjugate comprising the anti-CEACAM5 antibody may be administered at a dose level of about 120 mg/m², at day 1 on a first cycle of treatment, and the cycle may be about 2 weeks.

According to an embodiment, the antibody-drug conjugate comprising the anti-CEACAM5 antibody may be administered at a dose level of about 135 mg/m², at day 1 on a first cycle of treatment, and the cycle may be about 2 weeks.

According to an embodiment, the antibody-drug conjugate comprising the anti-CEACAM5 antibody may be administered at a dose level of about 150 mg/m², at day 1 on a first cycle of treatment, and the cycle may be about 2 weeks.

According to an embodiment, the antibody-drug conjugate comprising the anti-CEACAM5 antibody may be administered at a dose level of about 170 mg/m², at day 1 on a first cycle of treatment, and the cycle may be about 2 weeks.

According to an embodiment, the antibody-drug conjugate comprising the anti-CEACAM5 antibody may be administered at a dose of about 80 mg/m², at day 1 on a first cycle of treatment, as a loading dose, and at a dose of about 80 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at a dose of about 100 mg/m², at day 1 on a first cycle of treatment, as a loading dose, and at a dose of about 100 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at a dose of about 150 mg/m², at day 1 on a first cycle of treatment, as a loading dose, and at a dose of about 80 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at a dose of about 150 mg/m², at day 1 on a first cycle of treatment, as a loading dose, and at a dose of about 100 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at a dose of about 170 mg/m², at day 1 on a first cycle of treatment, as a loading dose, and at a dose of about 80 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at a dose of about 170 mg/m², at day 1 on a first cycle of treatment, as a loading dose, and at a dose of about 100 mg/m², as a subsequent dose, e.g., at day 1, on additional cycle(s). The cycle(s) may be about 2 weeks.

According to an embodiment, the antibody-drug conjugate comprising the anti-CEACAM5 antibody may be administered at dose of 170 mg/m², as a loading dose, on cycle 1, and at a dose of 100 mg/m², as a subsequent dose, on additional cycle(s). The cycle(s) may be about 2 or 3 weeks.

At day 1 of a first cycle of treatment the antibody-drug conjugate may be administered at a dose of about 80 mg/m², 100 mg/m², 120 mg/m², 135 mg/m², 150 mg/m² or about 170 mg/m², and the cycle of treatment may be 3 weeks.

According to an embodiment, the antibody-drug conjugate comprising the anti-CEACAM5 antibody may be administered at a dose of about 80 mg/m², at day 1 on a first cycle of treatment, and the cycle may be about 3 weeks.

According to an embodiment, the antibody-drug conjugate comprising the anti-CEACAM5 antibody may be administered at a dose of about 100 mg/m², at day 1 on a first cycle of treatment, and the cycle may be about 3 weeks.

According to an embodiment, the antibody-drug conjugate comprising the anti-CEACAM5 antibody may be administered at a dose level of about 120 mg/m², at day 1 on a first cycle of treatment, and the cycle may be about 3 weeks.

According to an embodiment, the antibody-drug conjugate comprising the anti-CEACAM5 antibody may be administered at a dose level of about 135 mg/m², at day 1 on a first cycle of treatment, and the cycle may be about 3 weeks.

According to an embodiment, the antibody-drug conjugate comprising the anti-CEACAM5 antibody may be administered at a dose level of about 150 mg/m², at day 1 on a first cycle of treatment, and the cycle may be about 3 weeks.

According to an embodiment, the antibody-drug conjugate comprising the anti-CEACAM5 antibody may be administered at a dose level of about 170 mg/m², at day 1 on a first cycle of treatment, and the cycle may be about 3 weeks.

According to an embodiment, the antibody-drug conjugate comprising the anti-CEACAM5 antibody may be administered at a dose of 100 mg/m² on all cycles, i.e., on cycle 1 and on additional cycle(s). The cycle(s) may be about 2 or 3 weeks.

According to an embodiment, the antibody-drug conjugate comprising the anti-CEACAM5 antibody may be administered at a dose of about 80 mg/m², at day 1 on a first cycle of treatment, as a loading dose, and at a dose of about 80 mg/m², as a subsequent dose, e.g., at day 1, on additional cycle(s). The cycle(s) may be about 3 weeks.

According to an embodiment, the antibody-drug conjugate comprising the anti-CEACAM5 antibody may be administered at a dose of about 100 mg/m², at day 1 on a first cycle of treatment, as a loading dose, and at a dose of about 100 mg/m², as a subsequent dose, e.g., at day 1, on additional cycle(s). The cycle(s) may be about 3 weeks.

According to an embodiment, the antibody-drug conjugate comprising the anti-CEACAM5 antibody may be administered at a dose of about 120 mg/m², at day 1 on a first cycle of treatment, as a loading dose, and at a dose of about 120 mg/m², as a subsequent dose, e.g., at day 1, on additional cycle(s). The cycle(s) may be about 3 weeks.

According to an embodiment, the antibody-drug conjugate comprising the anti-CEACAM5 antibody may be administered at a dose of about 135 mg/m², at day 1 on a first cycle of treatment, as a loading dose, and at a dose of about 135 mg/m², as a subsequent dose, e.g., at day 1, on additional cycle(s). The cycle(s) may be about 3 weeks.

According to an embodiment, the antibody-drug conjugate comprising the anti-CEACAM5 antibody may be administered at a dose of about 150 mg/m², at day 1 on a first cycle of treatment, as a loading dose, and at a dose of about 150 mg/m², as a subsequent dose, e.g., at day 1, on additional cycle(s). The cycle(s) may be about 3 weeks.

According to an embodiment, the antibody-drug conjugate comprising the anti-CEACAM5 antibody may be administered at a dose of about 170 mg/m², at day 1 on a first cycle of treatment, as a loading dose, and at a dose of about 170 mg/m², as a subsequent dose, e.g., at day 1, on additional cycle(s). The cycle(s) may be about 3 weeks.

The antibody-drug conjugate may be tusamitamab ravtansine (huMAb2-3-SPDB-DM4).

In some embodiments, the anti-VEGFR-2 antibody may be administered at a dose of from about 2 to about 20 mg/kg, or from about 4 to about 15 mg/kg, or from about 6 to about 10 mg/kg, or at about 8 mg/kg. The anti-VEGFR-2 antibody may be administered at a dose of about 2, 4, 6, 8, 10, 12, 14, 16, 18 or about 20 mg/kg.

In an embodiment, the anti-VEGFR-2 antibody may be administered at 8 mg/kg.

In an embodiment, the anti-VEGFR-2 antibody may be administered at 10 mg/kg.

In an embodiment, the anti-VEGFR-2 antibody may be administered at 8 mg/kg and the cycle may be about 2 weeks.

In an embodiment, the anti-VEGFR-2 antibody may be administered at 10 mg/kg and the cycle may be about 3 weeks.

The administered doses of the anti-VEGFR-2 antibody may be the same for a first cycle of treatment and the additional cycle(s).

On day 1 of a first cycle, the anti-VEGFR-2 antibody may be administered at a dose of about 8 mg/kg.

On day 1 of additional cycles (at least one cycle subsequent to the first cycle), the anti-VEGFR-2 antibody may be administered at a dose of about 8 mg/kg.

According to an embodiment, the anti-VEGFR-2 antibody may be administered at a dose of about 8 mg/kg, at day 1 on a first cycle of treatment, and the cycle may be about 2 or 3 weeks. In some embodiments, the cycle may be about 2 weeks.

According to an embodiment, the anti-VEGFR-2 antibody may be administered at a dose of about 10 mg/kg, at day 1 on a first cycle of treatment, and the cycle may be about 2 or 3 weeks. In some embodiments, the cycle may be about 3 weeks.

According to an embodiment, the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks.

According to an embodiment, the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks.

According to an embodiment, the anti-VEGFR-2 antibody may be administered at a dose of about 10 mg/kg, at day 1 on a first cycle of treatment, as a loading dose, and at a dose of about 8 mg/kg, as a subsequent dose, e.g., at day 1, on additional cycle(s). The cycle(s) may be about 2 or 3 weeks. The cycle(s) may be about 2 weeks.

The anti-VEGFR-2 antibody may be ramucirumab.

According to an embodiment, the antibody-drug conjugate comprising the anti-CEACAM5 antibody may be administered at a dose of about 80 mg/m², at day 1 on a first cycle of treatment, as a loading dose, and at a dose of about 80 mg/m², as a subsequent dose, e.g., at day 1, on additional cycle(s), and the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 100 mg/m², at day 1 on a first cycle of treatment, as a loading dose, and at a dose of about 100 mg/m², as a subsequent dose, e.g., at day 1, on additional cycle(s) and the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 150 mg/m², at day 1 on a first cycle of treatment, as a loading dose, and at a dose of about 80 mg/m², as a subsequent dose, e.g., at day 1, on additional cycle(s) the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 150 mg/m², at day 1 on a first cycle of treatment, as a loading dose, and at a dose of about 100 mg/m², as a subsequent dose, e.g., at day 1, on additional cycle(s) the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 170 mg/m², at day 1 on a first cycle of treatment, as a loading dose, and at a dose of about 80 mg/m², as a subsequent dose, e.g., at day 1, on additional cycle(s) and the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 170 mg/m², at day 1 on a first cycle of treatment, as a loading dose, and at a dose of about 100 mg/m², as a subsequent dose, e.g., at day 1, on additional cycle(s) and the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 80 mg/m², at day 1 on a first cycle of treatment, as a loading dose, and at a dose of about 80 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 100 mg/m², at day 1 on a first cycle of treatment, as a loading dose, and at a dose of about 100 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 120 mg/m², at day 1 on a first cycle of treatment, as a loading dose, and at a dose of about 120 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 135 mg/m², at day 1 on a first cycle of treatment, as a loading dose, and at a dose of about 135 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 150 mg/m², at day 1 on a first cycle of treatment, as a loading dose, and at a dose of about 150 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

According to an embodiment, the antibody-drug conjugate comprising the anti-CEACAM5 antibody may be administered at a dose of about 170 mg/m², at day 1 on a first cycle of treatment, as a loading dose, and at a dose of about 170 mg/m², as a subsequent dose, e.g., at day 1, on additional cycle(s) and the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks. The antibody-drug conjugate may be administered after the anti-VEGFR-2 antibody antibody-drug conjugate.

### Lung Cancer

In some embodiments, in a use for treating a lung cancer, such as a non-small cell lung cancer, such as non-squamous non-small-cell lung cancer (NSQ NSCLC), pharmaceutical compositions or combinations of the present disclosure may be such that in a first cycle of treatment, the antibody-drug conjugate comprising an anti-CEACAM5-antibody may be administered at a dose of from about 80 to about 170 mg/m², for example at about 80 mg/m², or at about 100 mg/m², or at about 120 mg/m², or at about 135 mg/m², or at about 150 mg/m², or at about 170 mg/m², and for example at about 100 mg/m². The pharmaceutical compositions or combinations of the present disclosure may be such that the anti-VEGFR-2 antibody may be administered at a dose from about 8 mg/kg to about 10 mg/kg.

In some embodiments, for example in a use for treating a lung cancer, such as a non-small cell lung cancer, such as non-squamous non-small-cell lung cancer (NSQ NSCLC), the antibody-drug conjugate comprising an anti-CEACAM5-antibody may be administered at a dose ranging from about 80 mg/m² to about 170 mg/m². The anti-VEGFR-2 antibody may be administered at a dose from about 8 mg/kg to about 10 mg/kg.

In various embodiments, the antibody-drug conjugate comprising the anti-CEACAM5 antibody may be administered at a dose of about 80, 90, 100, 110, 120, 130, 135, 140, 145, 150, 155, 160, 165, or about 170 mg/m².

In various embodiments, the anti-VEGFR-2 antibody may be administered at a dose of about 8, 8.5, 9, 9.5, or about 10 mg/kg.

In a use for treating a lung cancer, in a first cycle of treatment, for example as above indicated, the antibody-drug conjugate comprising an anti-CEACAM5-antibody may be administered at a dose of about 80 mg/m² or about 100 mg/m² or about 170 mg/m². Such a dose may be a loading dose or a first dose.

In a use for treating a lung cancer, such as a non-small cell lung cancer, such as non-squamous non-small-cell lung cancer (NSQ NSCLC), the pharmaceutical compositions or combinations of the present disclosure may be such that, in a first cycle of treatment, for example as above indicated, the antibody-drug conjugate comprising an anti-CEACAM5-antibody may be administered at a dose of about 80 mg/m² or about 100 mg/m², or about 120 mg/m², or about 135 mg/m², or about 150 mg/m², or about 170 mg/m².

In a use for treating a lung cancer, at day 1 of a first cycle of treatment the antibody-drug conjugate may be administered at a dose of 80 mg/m², 100 mg/m², 120 mg/m², 135 mg/m² or 150 mg/m². Such a dose may be a loading dose.

In a use for treating a lung cancer, at day 1 of a first cycle of treatment the antibody-drug conjugate may be administered at a dose of 80 mg/m² or 100 mg/m², and the cycle of treatment may be 2 weeks.

In a use for treating a lung cancer, at day 1 of a first cycle of treatment the antibody-drug conjugate may be administered at a dose of 120 mg/m², 135 mg/m², or 150 mg/m², and the cycle of treatment may be 3 weeks.

In a use for treating a lung cancer, at day 1 of a first cycle of treatment the anti-VEGFR-2 antibody may be administered at a dose of 8 mg/kg, and the cycle may be 2 weeks.

In a use for treating a lung cancer, at day 1 of a first cycle of treatment the anti-VEGFR-2 antibody may be administered at a dose of 10 mg/kg, and the cycle may be 3 weeks.

In some embodiment, the pharmaceutical compositions or combinations of the present disclosure may be such that the antibody-drug conjugate comprising an anti-CEACAM5-antibody may be administered at a dose of about 80 mg/m². The administration may be carried out on the first day (at day 1) of a first cycle of treatment. A cycle may last about 2 weeks.

In some embodiment, the pharmaceutical compositions or combinations of the present disclosure may be such that the antibody-drug conjugate comprising an anti-CEACAM5-antibody may be administered at a dose of about 100 mg/m². The administration may be carried out on the first day (at day 1) of a first cycle of treatment. A cycle may last about 2 weeks.

In some embodiment, the pharmaceutical compositions or combinations of the present disclosure may be such that the antibody-drug conjugate comprising an anti-CEACAM5-antibody may be administered at a dose of about 120 mg/m². The administration may be carried out on the first day (at day 1) of a first cycle of treatment. A cycle may last about 3 weeks.

In some embodiment, the pharmaceutical compositions or combinations of the present disclosure may be such that the antibody-drug conjugate comprising an anti-CEACAM5-antibody may be administered at a dose of about 135 mg/m². The administration may be carried out on the first day (at day 1) of a first cycle of treatment. A cycle may last about 3 weeks.

In some embodiment, the pharmaceutical compositions or combinations of the present disclosure may be such that the antibody-drug conjugate comprising an anti-CEACAM5-antibody may be administered at a dose of about 150 mg/m². The administration may be carried out on the first day (at day 1) of a first cycle of treatment. A cycle may last about 3 weeks.

In some embodiment, the pharmaceutical compositions or combinations of the present disclosure may be such that, the antibody-drug conjugate comprising an anti-CEACAM5-antibody may be administered at a dose of about 170 mg/m². The administration may be carried out on the first day (at day 1) of a first cycle of treatment. A cycle may be about 3 weeks.

As above indicated, a use for treating a lung cancer, such as a non-small cell lung cancer, such as non-squamous non-small-cell lung cancer (NSQ NSCLC), may comprise, further to a first cycle of treatment, at least one additional cycle of treatment. The pharmaceutical compositions or combinations of the present disclosure may be such that the antibody-drug conjugate comprising an anti-CEACAM5-antibody may be administered, in additional cycle(s) of treatment, at a dose of about 80 mg/m² or about 100 mg/m² about 120 mg/m² or about 135 mg/m² or about 150 mg/m² or 170 mg/m². The administration may be carried out on the first day (at day 1) of the additional cycle(s) of treatment. Such a dose may be a subsequent dose.

At day 1 of an at least one additional cycle of treatment the antibody-drug conjugate may be administered at a dose of 80 mg/m², 100 mg/m², 120 mg/m², 135 mg/m², or 150 mg/m².

At day 1 of an at least one additional cycle of treatment the antibody-drug conjugate may be administered at a dose of 80 mg/m² or 100 mg/m², and the cycle may be 2 weeks.

At day 1 of an at least one additional cycle of treatment the antibody-drug conjugate may be administered at a dose of 120 mg/m², 135 mg/m² or 150 mg/m², and the cycle last 3 weeks.

At day 1 of an at least one additional cycle of treatment the anti-VEGFR-2 antibody may be administered at a dose of 8 mg/kg, and the cycle may be 2 weeks.

At day 1 of an at least one additional cycle of treatment the anti-VEGFR-2 antibody may be administered at a dose of 10 mg/kg, and the cycle may be 3 weeks.

In some embodiment, in a use for treating a lung cancer, the antibody-drug conjugate comprising an anti-CEACAM5-antibody may be administered at a dose of about 80 mg/m². The administration may be carried out on the first day (at day 1) of additional cycle(s) of treatment. A cycle may last about 2 weeks.

In some embodiment, in a use for treating a lung cancer, the antibody-drug conjugate comprising an anti-CEACAM5-antibody may be administered at a dose of about 100 mg/m². The administration may be carried out on the first day (at day 1) of additional cycle(s) of treatment. A cycle may last about 2 weeks.

In some embodiment, in a use for treating a lung cancer, the antibody-drug conjugate comprising an anti-CEACAM5-antibody may be administered at a dose of about 120 mg/m². The administration may be carried out on the first day (at day 1) of additional cycle(s) of treatment. A cycle may last about 3 weeks.

In some embodiment, in a use for treating a lung cancer, the antibody-drug conjugate comprising an anti-CEACAM5-antibody may be administered at a dose of about 135 mg/m². The administration may be carried out on the first day (at day 1) of additional cycle(s) of treatment. A cycle may last about 3 weeks.

In some embodiment, in a use for treating a lung cancer, the antibody-drug conjugate comprising an anti-CEACAM5-antibody may be administered at a dose of about 150 mg/m². The administration may be carried out on the first day (at day 1) of additional cycle(s) of treatment. A cycle may last about 3 weeks.

In some embodiment, the antibody-drug conjugate comprising an anti-CEACAM5-antibody may be administered at a dose of about 170 mg/m². The administration may be carried out on the first day (at day 1) of additional cycle(s) of treatment. A cycle may last about 3 weeks.

In some embodiment, the pharmaceutical compositions or combinations of the present disclosure may be such that the antibody-drug conjugate may be administered at a dose of about 80 mg/m², on the first day (at day 1) of a first cycle of treatment, and at a dose of about 80 mg/m², on the first of day of additional cycle(s). A cycle may last two weeks.

In some embodiment, the pharmaceutical compositions or combinations of the present disclosure may be such that the antibody-drug conjugate may be administered at a dose of about 100 mg/m², on the first day (at day 1) of a first cycle of treatment, and at a dose of about 100 mg/m², on the first of day of additional cycle(s). A cycle may last two weeks.

In some embodiment, the pharmaceutical compositions or combinations of the present disclosure may be such that the antibody-drug conjugate may be administered at a dose of about 120 mg/m², on the first day (at day 1) of a first cycle of treatment, and at a dose of about 120 mg/m², on the first of day of additional cycle(s). A cycle may last three weeks.

In some embodiment, the pharmaceutical compositions or combinations of the present disclosure may be such that the antibody-drug conjugate may be administered at a dose of about 135 mg/m², on the first day (at day 1) of a first cycle of treatment, and at a dose of about 135 mg/m², on the first of day of additional cycle(s). A cycle may last three weeks.

In some embodiment, the pharmaceutical compositions or combinations of the present disclosure may be such that the antibody-drug conjugate may be administered at a dose of about 150 mg/m², on the first day (at day 1) of a first cycle of treatment, and at a dose of about 150 mg/m², on the first of day of additional cycle(s). A cycle may last three weeks.

In some embodiment, the pharmaceutical compositions or combinations of the present disclosure may be such that the antibody-drug conjugate comprising an anti-CEACAM5-antibody may be administered at a dose of about 170 mg/m², on the first day (at day 1) of a first cycle of treatment, and at a dose of about 100 or 170 mg/m², on the first of day of additional cycle(s). A cycle may last three weeks.

In a use for treating a lung cancer, in a first cycle of treatment and/or in additional cycles of treatment, for example as above indicated, the pharmaceutical compositions or combinations of the present disclosure may be such that the anti-VEGFR-2 antibody may be administered at a dose of about 8 mg/kg or about 10 mg/kg. In some embodiment, the pharmaceutical compositions or combinations of the present disclosure may be such that the anti-VEGFR-2 antibody may be administered at a dose of about 8 mg/kg. In some embodiment, the pharmaceutical compositions or combinations of the present disclosure may be such that the anti-VEGFR-2 antibody may be administered at a dose of about 10 mg/kg. The administration may be carried out on the first day (at day 1) of the cycles (first and additional) of treatment.

In some embodiment, the cycle may be about 2 or 3 weeks.

In some embodiment, the pharmaceutical compositions or combinations of the present disclosure may be such that the anti-VEGFR-2 antibody may be administered at a dose of about 8 mg/kg and the cycle may be of 2 weeks.

In some embodiment, the pharmaceutical compositions or combinations of the present disclosure may be such that the anti-VEGFR-2 antibody may be administered at a dose of about 10 mg/kg and the cycle may be of 3 weeks.

The administered doses of the anti-VEGFR-2 antibody may be the same for a first cycle of treatment and the additional cycle(s).

On day 1 of a first cycle, the anti-VEGFR-2 antibody may be administered at a dose of about 8 mg/kg.

On day 1 of additional cycles (at least one cycle subsequent to the first cycle), the anti-VEGFR-2 antibody may be administered at a dose of about 8 mg/kg.

According to an embodiment, the anti-VEGFR-2 antibody may be administered at a dose of about 8 mg/kg, at day 1 on a first cycle of treatment, and the cycle may be about 2 or 3 weeks. In some embodiments, the cycle may be about 2 weeks.

According to an embodiment, the anti-VEGFR-2 antibody may be administered at a dose of about 10 mg/kg, at day 1 on a first cycle of treatment, and the cycle may be about 2 or 3 weeks. In some embodiments, the cycle may be about 3 weeks.

According to an embodiment, the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks.

According to an embodiment, the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks.

According to an embodiment, the anti-VEGFR-2 antibody may be administered at a dose of about 10 mg/kg, at day 1 on a first cycle of treatment, as a loading dose, and at a dose of about 8 mg/kg, as a subsequent dose, e.g., at day 1, on additional cycle(s). The cycle(s) may be about 2 or 3 weeks. The cycle(s) may be about 2 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, in first and additional cycles of treatment, the antibody-drug conjugate comprising an anti-CEACAM5-antibody may be administered at a dose of from about 80 to about 170 mg/m², for example at about 100 mg/m², and the anti-VEGFR-2 antibody may be administered at a dose of about 8 or about 10 mg/kg. In some embodiment, the cycle may be about 2 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, at day 1 of a first cycle and at day 1 of an at least one additional cycle of treatment, the antibody-drug conjugate may be administered at a dose of 80 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, at day 1 of a first cycle and at day 1 of an at least one additional cycle of treatment, the antibody-drug conjugate may be administered at a dose of 100 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, in first and additional cycles of treatment, the antibody-drug conjugate comprising an anti-CEACAM5-antibody may be administered at a dose of from about 80 to about 170 mg/m², for example at about 170 mg/m², and the anti-VEGFR-2 antibody may be administered at a dose of about 8 or 10 mg/kg. In some embodiment, the cycle may be about 3 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, at day 1 of a first cycle and at day 1 of an at least one additional cycle of treatment, the antibody-drug conjugate may be administered at a dose of 120 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, at day 1 of a first cycle and at day 1 of an at least one additional cycle of treatment, the antibody-drug conjugate may be administered at a dose of 135 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that, at day 1 of a first cycle and at day 1 of an at least one additional cycle of treatment, the antibody-drug conjugate may be administered at a dose of 150 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 80 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 100 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 120 mg/m², at day 1 on a first cycle of treatment, as a loading dose, and at a dose of about 120 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 135 mg/m², at day 1 on a first cycle of treatment, as a loading dose, and at a dose of about 135 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 150 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a lung cancer, such as a non-small cell lung cancer, such as non-squamous non-small-cell lung cancer (NSQ NSCLC), pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 170 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiment, a combination of an antibody-drug conjugate comprising an anti-CEACAM5-antibody and an anti-VEGFR-2 antibody may be for use in a lung cancer, such as a non-small cell lung cancer, such as non-squamous non-small-cell lung cancer (NSQ NSCLC), in a patient, wherein the patient has a CEACAM5-positive cancer (defined as a cancer having a CEACAM5 immunohistochemical [IHC] intensity ≥2+ in ≥50% of cancer cells or ≥2+ intensity in ≥1% and <50% of cancer cells).

In some embodiment, a combination of an antibody-drug conjugate comprising an anti-CEACAM5-antibody and an anti-VEGFR-2 antibody may be for use in treating a lung cancer, such as a non-small cell lung cancer, such as non-squamous non-small-cell lung cancer (NSQ NSCLC), in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg and subsequently the antibody-drug conjugate comprising an anti-CEACAM5-antibody is administered at a dose of 80 mg/m², on day 1 of a first cycle, and the cycle is 2 weeks.

In some embodiment, the anti-VEGFR-2 antibody is administered at a dose of 8 mg/kg as a subsequent dose, and subsequently the antibody-drug conjugate comprising an anti-CEACAM5-antibody is administered at a dose of 80 mg/m² as a subsequent dose, on day 1 of additional cycle(s), and the cycle(s) is/are 2 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a lung cancer, such as a non-small cell lung cancer, such as non-squamous non-small-cell lung cancer (NSQ NSCLC), in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg and subsequently the antibody-drug conjugate comprising an anti-CEACAM5-antibody is administered at a dose of 100 mg/m², on day 1 of a first cycle, and the cycle is 2 weeks.

In some embodiment, the anti-VEGFR-2 antibody is administered at a dose of 8 mg/kg as a subsequent dose, and subsequently the antibody-drug conjugate comprising an anti-CEACAM5-antibody is administered at a dose of 100 mg/m² as a subsequent dose, on day 1 of additional cycle(s), and the cycle(s) is/are 2 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a lung cancer, such as a non-small cell lung cancer, such as non-squamous non-small-cell lung cancer (NSQ NSCLC), in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg and subsequently the antibody-drug conjugate comprising an anti-CEACAM5-antibody is administered at a dose of 120 mg/m², on day 1 of a first cycle, and the cycle is 3 weeks.

In some embodiment, the anti-VEGFR-2 antibody is administered at a dose of 10 mg/kg as a subsequent dose, and sequentially the antibody-drug conjugate comprising an anti-CEACAM5-antibody is administered at a dose of 120 mg/m² as a subsequent dose, on day 1 of additional cycle(s), and the cycle is 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a lung cancer, such as a non-small cell lung cancer, such as non-squamous non-small-cell lung cancer (NSQ NSCLC), in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg and subsequently the antibody-drug conjugate comprising an anti-CEACAM5-antibody is administered at a dose of 135 mg/m², on day 1 of a first cycle, and the cycle is 3 weeks.

In some embodiment, the anti-VEGFR-2 antibody is administered at a dose of 10 mg/kg as a subsequent dose, and sequentially the antibody-drug conjugate comprising an anti-CEACAM5-antibody is administered at a dose of 135 mg/m² as a subsequent dose, on day 1 of additional cycle(s), and the cycle is 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a lung cancer, such as a non-small cell lung cancer, such as non-squamous non-small-cell lung cancer (NSQ NSCLC), in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg and subsequently the antibody-drug conjugate comprising an anti-CEACAM5-antibody is administered at a dose of 150 mg/m², on day 1 of a first cycle, and the cycle is 3 weeks.

In some embodiment, the anti-VEGFR-2 antibody is administered at a dose of 10 mg/kg as a subsequent dose, and sequentially the antibody-drug conjugate comprising an anti-CEACAM5-antibody is administered at a dose of 150 mg/m² as a subsequent dose, on day 1 of additional cycle(s), and the cycle is 3 weeks.

In some embodiment, the patient has a CEACAM5-positive cancer having a CEACAM5 immunohistochemical intensity ≥2+ in ≥50% of cancer cells.

In certain embodiments, the patient has a cancer having a high CEACAM5 expression on tumor cells. A high CEACAM5 expression on tumor cells may be defined as being ≥2+ intensity in ≥50% of cancer cells, as measured by immunohistochemistry (IHC).

The lung cancer may be a non-squamous non-small-cell lung cancer (NSQ NSCLC).

In certain embodiments, for patients with a body surface area (BSA) >2.2 m², the dose of the antibody-drug conjugate comprising an anti-CEACAM5-antibody may be calculated based on a BSA of 2.2 m².

The lung cancer may be a non-squamous non-small-cell lung cancer (NSQ NSCLC).

In some embodiments, the dosage regimen comprises administration of the dose over a period of about 10 minutes to about 48 hours, or of about 1 h to about 48 h, such as over a period of 1 h to 4 h. In an aspect of this embodiment, the dose frequency varies from twice a week to once every three weeks, for example every 2 weeks or every 3 weeks.

In an embodiment, the treatment duration is of at least 4 or 6 months.

### Gastric Cancer and Gastroesophageal Adenocarcinoma (GEJ) Cancer

In some embodiments, for example in a use for treating a gastric cancer (GC) or a gastroesophageal adenocarcinoma (GEJ) cancer the antibody-drug conjugate comprising an anti-CEACAM5-antibody may be administered at a dose ranging from about 80 mg/m² to about 170 mg/m² or from about 100 mg/m² to about 170 mg/m², for example at a dose of about 150 mg/m² to about 170 mg/m². The anti-VEGFR-2 antibody may be administered at a dose from about 8 mg/kg to about 10 mg/kg.

In various embodiments, the antibody-drug conjugate comprising the anti-CEACAM5 antibody may be administered at a dose of about 80, 100, 110, 120, 130, 135, 140, 145, 150, 155, 160, 165, or about 170 mg/m².

In various embodiments, the anti-VEGFR-2 antibody may be administered at a dose of about 8, 8.5, 9, 9.5, or about 10 mg/kg.

In a use for treating a gastric cancer (GC) or a gastroesophageal adenocarcinoma (GEJ) cancer, at day 1 of a first cycle of treatment the anti-VEGFR-2 antibody may be administered at a dose of 8 mg/kg, and the cycle may be 2 weeks.

In a use for treating a gastric cancer (GC) or a gastroesophageal adenocarcinoma (GEJ) cancer, at day 1 of a first cycle of treatment the anti-VEGFR-2 antibody may be administered at a dose of 10 mg/kg, and the cycle may be 3 weeks.

In a use for treating a gastric cancer (GC) or a gastroesophageal adenocarcinoma (GEJ) cancer, in a first cycle of treatment, for example as above indicated, the antibody-drug conjugate comprising an anti-CEACAM5-antibody may be administered at a dose of about 80 mg/m², 100 mg/m², 120 mg/m², 135 mg/m², 150 mg/m² or about 170 mg/m², as a loading dose.

In a use for treating a gastric cancer (GC) or a gastroesophageal adenocarcinoma (GEJ) cancer, in a first cycle of treatment, for example as above indicated, the antibody-drug conjugate comprising an anti-CEACAM5-antibody may be administered at a dose of about 120 mg/m², 135 mg/m², about 150 mg/m² or about 170 mg/m², as a loading dose.

In a use for treating a gastric cancer (GC) or a gastroesophageal adenocarcinoma (GEJ) cancer, at day 1 of a first cycle of treatment the antibody-drug conjugate is administered at a dose of 80 mg/m², 100 mg/m², 120 mg/m², 135 mg/m², 150 mg/m² or 170 mg/m². Such a dose may be a loading dose. The cycle of treatment may be 2 or 3 weeks In some embodiment, the antibody-drug conjugate comprising an anti-CEACAM5-antibody may be administered at a dose of about 100 mg/m². The administration may be carried out on the first day (at day 1) of a first cycle of treatment. Such a dose may be a loading dose. The cycle of treatment may be 2 or 3 weeks.

In some embodiment, the antibody-drug conjugate comprising an anti-CEACAM5-antibody may be administered at a dose of about 120 mg/m². The administration may be carried out on the first day (at day 1) of a first cycle of treatment. Such a dose may be a loading dose. The cycle of treatment may be 2 or 3 weeks.

In some embodiment, the antibody-drug conjugate comprising an anti-CEACAM5-antibody may be administered at a dose of about 135 mg/m². The administration may be carried out on the first day (at day 1) of a first cycle of treatment. Such a dose may be a loading dose. The cycle of treatment may be 2 or 3 weeks.

In some embodiment, the antibody-drug conjugate comprising an anti-CEACAM5-antibody may be administered at a dose of about 150 mg/m². The administration may be carried out on the first day (at day 1) of a first cycle of treatment. Such a dose may be a loading dose. The cycle of treatment may be 2 or 3 weeks.

In some embodiment, the antibody-drug conjugate comprising an anti-CEACAM5-antibody may be administered at a dose of about 170 mg/m². The administration may be carried out on the first day (at day 1) of a first cycle of treatment. Such a dose may be a loading dose. The cycle of treatment may be 2 or 3 weeks.

As above indicated, a use for treating a gastric cancer (GC) or GEJ cancer may comprise, further to a first cycle of treatment, at least one additional cycle of treatment. The pharmaceutical compositions or combinations of the present disclosure may be such that the antibody-drug conjugate comprising an anti-CEACAM5-antibody may be administered, in additional cycle(s) of treatment, at a dose of about 80 mg/m² or about 100 mg/m² about 120 mg/m² or about 135 mg/m² or about 150 mg/m² or 170 mg/m². The administration may be carried out on the first day (at day 1) of the additional cycle(s) of treatment. Such a dose may be a subsequent dose.

At day 1 of an at least one additional cycle of treatment the antibody-drug conjugate may be administered at a dose of 80 mg/m², 100 mg/m², 120 mg/m², 135 mg/m², or 150 mg/m².

At day 1 of an at least one additional cycle of treatment the antibody-drug conjugate may be administered at a dose of 80 mg/m² or 100 mg/m², and the cycle may be 2 weeks.

At day 1 of an at least one additional cycle of treatment the antibody-drug conjugate may be administered at a dose of 120 mg/m², 135 mg/m² or 150 mg/m², and the cycle last 3 weeks.

At day 1 of an at least one additional cycle of treatment the anti-VEGFR-2 antibody may be administered at a dose of 8 mg/kg, and the cycle may be 2 weeks.

At day 1 of an at least one additional cycle of treatment the anti-VEGFR-2 antibody may be administered at a dose of 10 mg/kg, and the cycle may be 3 weeks.

The antibody-drug conjugate comprising an anti-CEACAM5-antibody may be administered, in additional cycle(s) of treatment, at a dose of about 80 mg/m², i.e., as a subsequent dose. The administration may be carried out on the first day (at day 1) of the additional cycle(s) of treatment. The cycle of treatment may be 2 or 3 weeks.

The antibody-drug conjugate comprising an anti-CEACAM5-antibody may be administered, in additional cycle(s) of treatment, at a dose of about 100 mg/m², i.e., as a subsequent dose. The administration may be carried out on the first day (at day 1) of the additional cycle(s) of treatment. The cycle of treatment may be 2 or 3 weeks.

The antibody-drug conjugate comprising an anti-CEACAM5-antibody may be administered, in additional cycle(s) of treatment, at a dose of about 120 mg/m², i.e., as a subsequent dose. The administration may be carried out on the first day (at day 1) of the additional cycle(s) of treatment. The cycle of treatment may be 2 or 3 weeks.

The antibody-drug conjugate comprising an anti-CEACAM5-antibody may be administered, in additional cycle(s) of treatment, at a dose of about 135 mg/m², i.e., as a subsequent dose. The administration may be carried out on the first day (at day 1) of the additional cycle(s) of treatment. The cycle of treatment may be 2 or 3 weeks.

The antibody-drug conjugate comprising an anti-CEACAM5-antibody may be administered, in additional cycle(s) of treatment, at a dose of about 150 mg/m², i.e., as a subsequent dose. The administration may be carried out on the first day (at day 1) of the additional cycle(s) of treatment. The cycle of treatment may be 2 or 3 weeks.

The antibody-drug conjugate comprising an anti-CEACAM5-antibody may be administered, in additional cycle(s) of treatment, at a dose of about 170 mg/m², i.e., as a subsequent dose. The administration may be carried out on the first day (at day 1) of the additional cycle(s) of treatment. The cycle of treatment may be 2 or 3 weeks.

In some embodiment, for treating a gastric cancer (GC) or GEJ cancer, the antibody-drug conjugate comprising an anti-CEACAM5-antibody may be administered at a dose of about 80 mg/m², on the first day (at day 1) of a first cycle of treatment, and at a dose of about 80 mg/m², on the first of day of additional cycle(s). A cycle may be three weeks.

In some embodiment, for treating a gastric cancer (GC) or GEJ cancer, the antibody-drug conjugate comprising an anti-CEACAM5-antibody may be administered at a dose of about 100 mg/m², on the first day (at day 1) of a first cycle of treatment, and at a dose of about 100 mg/m², on the first of day of additional cycle(s). A cycle may be three weeks.

In some embodiment, for treating a gastric cancer (GC) or GEJ cancer, the antibody-drug conjugate comprising an anti-CEACAM5-antibody may be administered at a dose of about 120 mg/m², on the first day (at day 1) of a first cycle of treatment, and at a dose of about 120 mg/m², on the first of day of additional cycle(s). A cycle may be three weeks.

In some embodiment, for treating a gastric cancer (GC) or GEJ cancer, the antibody-drug conjugate comprising an anti-CEACAM5-antibody may be administered at a dose of about 135 mg/m², on the first day (at day 1) of a first cycle of treatment, and at a dose of about 135 mg/m², on the first of day of additional cycle(s). A cycle may be three weeks.

In some embodiment, for treating a gastric cancer (GC) or GEJ cancer, the antibody-drug conjugate comprising an anti-CEACAM5-antibody may be administered at a dose of about 150 mg/m², on the first day (at day 1) of a first cycle of treatment, and at a dose of about 80 mg/m², on the first of day of additional cycle(s). A cycle may be two weeks.

In some embodiment, for treating a gastric cancer (GC) or GEJ cancer, the antibody-drug conjugate comprising an anti-CEACAM5-antibody may be administered at a dose of about 150 mg/m², on the first day (at day 1) of a first cycle of treatment, and at a dose of about 100 mg/m², on the first of day of additional cycle(s). A cycle may be two weeks.

In some embodiment, for treating a gastric cancer (GC) or GEJ cancer, the antibody-drug conjugate comprising an anti-CEACAM5-antibody may be administered at a dose of about 150 mg/m², on the first day (at day 1) of a first cycle of treatment, and at a dose of about 150 mg/m², on the first of day of additional cycle(s). A cycle may be three weeks.

In some embodiment, for treating a gastric cancer (GC) or GEJ cancer, the antibody-drug conjugate comprising an anti-CEACAM5-antibody may be administered at a dose of about 170 mg/m², on the first day (at day 1) of a first cycle of treatment, and at a dose of about 80 mg/m², on the first of day of additional cycle(s). A cycle may be two weeks.

In some embodiment, for treating a gastric cancer (GC) or GEJ cancer, the antibody-drug conjugate comprising an anti-CEACAM5-antibody may be administered at a dose of about 170 mg/m², on the first day (at day 1) of a first cycle of treatment, and at a dose of about 100 mg/m², on the first of day of additional cycle(s). A cycle may be two weeks or three weeks. A cycle may be two weeks.

In some embodiment, for treating a gastric cancer (GC) or GEJ cancer, the antibody-drug conjugate comprising an anti-CEACAM5-antibody may be administered at a dose of about 170 mg/m², on the first day (at day 1) of a first cycle of treatment, and at a dose of about 170 mg/m², on the first of day of additional cycle(s). A cycle may be three weeks.

In a use for treating a gastric cancer (GC) or GEJ cancer, in a first cycle of treatment and/or in additional cycles of treatment, for example as above indicated, the anti-VEGFR-2 antibody may be administered at a dose of about 8 mg/kg or of about 10 mg/kg. In some embodiment, the anti-VEGFR-2 antibody may be administered at a dose of about 8 mg/kg. The administration may be carried out on the first day (at day 1) of the cycles of treatment. In some embodiment, the pharmaceutical compositions or combinations of the present disclosure may be such that the anti-VEGFR-2 antibody may be administered at a dose of about 10 mg/kg. The administration may be carried out on the first day (at day 1) of the cycles (first and additional) of treatment.

In some embodiment, the cycles are of 2 or 3 weeks.

In some embodiment, the pharmaceutical compositions or combinations of the present disclosure may be such that the anti-VEGFR-2 antibody may be administered at a dose of about 8 mg/kg and the cycle may be of 2 weeks.

In some embodiment, the pharmaceutical compositions or combinations of the present disclosure may be such that the anti-VEGFR-2 antibody may be administered at a dose of about 10 mg/kg and the cycle may be of 3 weeks.

The administered doses of the anti-VEGFR-2 antibody may be the same for a first cycle of treatment and the additional cycle(s).

On day 1 of a first cycle, the anti-VEGFR-2 antibody may be administered at a dose of about 8 mg/kg.

On day 1 of additional cycles (at least one cycle subsequent to the first cycle), the anti-VEGFR-2 antibody may be administered at a dose of about 8 mg/kg.

According to an embodiment, the anti-VEGFR-2 antibody may be administered at a dose of about 8 mg/kg, at day 1 on a first cycle of treatment, and the cycle may be about 2 or 3 weeks. In some embodiments, the cycle may be about 2 weeks.

According to an embodiment, the anti-VEGFR-2 antibody may be administered at a dose of about 10 mg/kg, at day 1 on a first cycle of treatment, and the cycle may be about 2 or 3 weeks. In some embodiments, the cycle may be about 3 weeks.

According to an embodiment, the anti-VEGFR-2 antibody may be administered at a dose of about 8 mg/kg, at day 1 on a first cycle of treatment, as a loading dose, and at a dose of about 8 mg/kg, as a subsequent dose, e.g., at day 1, on additional cycle(s). The cycle(s) may be about 2 weeks.

According to an embodiment, the anti-VEGFR-2 antibody may be administered at a dose of about 10 mg/kg, at day 1 on a first cycle of treatment, as a loading dose, and at a dose of about 10 mg/kg, as a subsequent dose, e.g., at day 1, on additional cycle(s). The cycle(s) may be about 3 weeks.

According to an embodiment, the anti-VEGFR-2 antibody may be administered at a dose of about 10 mg/kg, at day 1 on a first cycle of treatment, as a loading dose, and at a dose of about 8 mg/kg, as a subsequent dose, e.g., at day 1, on additional cycle(s). The cycle(s) may be about 2 or 3 weeks. The cycle(s) may be about 2 weeks.

In some embodiments, in a use for treating a gastric cancer (GC) or GEJ cancer, pharmaceutical compositions or combinations of the present disclosure may be such that, in first and additional cycles of treatment, the antibody-drug conjugate comprising an anti-CEACAM5-antibody may be administered at a dose of from about 80 to about 170 mg/m², for example at about 100 mg/m², and the anti-VEGFR-2 antibody may be administered at a dose of about 8 or about 10 mg/kg. In some embodiment, the cycle may be about 2 or about 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 80 mg/m² and the anti-VEGFR-2 antibody may be administered at a dose of 8 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 100 mg/m² and the anti-VEGFR-2 antibody may be administered at a dose of 8 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 120 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 135 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle, the antibody-drug conjugate may be administered at a dose of 150 mg/m² and the anti-VEGFR-2 antibody may be administered at a dose of 8 mg/kg, and at day 1 of an at least one additional cycle of treatment the antibody-drug conjugate may be administered at a dose of 80 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle, the antibody-drug conjugate may be administered at a dose of 150 mg/m² and the anti-VEGFR-2 antibody may be administered at a dose of 8 mg/kg, and at day 1 of an at least one additional cycle of treatment the antibody-drug conjugate may be administered at a dose of 100 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 150 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle, the antibody-drug conjugate may be administered at a dose of 170 mg/m² and the anti-VEGFR-2 antibody may be administered at a dose of 8 mg/kg, and at day 1 of an at least one additional cycle of treatment the antibody-drug conjugate may be administered at a dose of 80 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle, the antibody-drug conjugate may be administered at a dose of 170 mg/m² and the anti-VEGFR-2 antibody may be administered at a dose of 8 mg/kg, and at day 1 of an at least one additional cycle of treatment the antibody-drug conjugate may be administered at a dose of 100 mg/m² and the anti-VEGFR-2 antibody may be administered at a dose of 8 mg/kg, and the cycle of treatment may be 2 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that, at day 1 of a first cycle and at day 1 of an at least one additional cycle of treatment, the antibody-drug conjugate may be administered at a dose of 170 mg/m² and the anti-VEGFR-2 antibody may be administered at a dose of 10 mg/kg, and the cycle of treatment may be 3 weeks. The antibody-drug conjugate may be administered after the anti-VEGFR-2 antibody antibody-drug conjugate.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 80 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 3 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 100 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 3 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 120 mg/m², at day 1 on a first cycle of treatment, as a loading dose, and at a dose of about 120 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 135 mg/m², at day 1 on a first cycle of treatment, as a loading dose, and at a dose of about 135 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 150 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

In some embodiments, in a use for treating a gastric cancer (GC) or GEJ cancer, pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 170 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer (defined as a cancer having a CEACAM5 immunohistochemical [IHC] intensity ≥2+ in ≥50% of cancer cells or ≥2+ intensity in ≥1% and <50% of cancer cells).

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg, and subsequently the antibody-drug conjugate comprising an anti-CEACAM5-antibody may be administered at a dose of 150 mg/m² on day 1 of a first cycle, and the cycle may be 2 weeks.

In some embodiment, the anti-VEGFR-2 antibody may be administered at a dose of 8 mg/kg as a subsequent dose, and subsequently the antibody-drug conjugate comprising an anti-CEACAM5-antibody may be administered at a dose of 80 mg/m² as a subsequent dose, on day 1 of additional cycle(s), and the cycle(s) may be 2 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 8 mg/kg, and subsequently the antibody-drug conjugate comprising an anti-CEACAM5-antibody may be administered at a dose of 170 mg/m², on day 1 of a first cycle, and the cycle may be 2 weeks.

In some embodiment, the anti-VEGFR-2 antibody may be administered at a dose of 8 mg/kg as a subsequent dose, and subsequently the antibody-drug conjugate comprising an anti-CEACAM5-antibody may be administered at a dose of 100 mg/m² as a subsequent dose, on day 1 of additional cycle(s), and the cycle(s) may be 2 weeks

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 80 mg/m², 100 mg/m², 120 mg/m², 150 mg/m² or 170 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 80 mg/m², 100 mg/m², 120 mg/m², 150 mg/m² or 170 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 80 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 80 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 100 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 100 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 120 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 120 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 135 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 135 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 150 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 150 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In some embodiment, a combination of an antibody-drug conjugate comprising an anti-CEACAM5-antibody and an anti-VEGFR-2 antibody may be for use in treating a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody may be administered at a dose of 10 mg/kg, and subsequently the antibody-drug conjugate comprising an anti-CEACAM5-antibody may be administered at a dose of 170 mg/m², on day 1 of a first cycle, and the cycle may be 3 weeks.

In some embodiment, the anti-VEGFR-2 antibody may be administered at a dose of 10 mg/kg as a subsequent dose, and subsequently the antibody-drug conjugate comprising an anti-CEACAM5-antibody may be administered at a dose of 170 mg/m² as a subsequent dose, on day 1 of additional cycle(s), and the cycle(s) may be 3 weeks.

In some embodiment, the patient has a CEACAM5-positive cancer having a CEACAM5 immunohistochemical intensity ≥2+ in ≥50% of cancer cells.

In certain embodiments, the patient has a cancer having a high CEACAM5 expression on tumor cells. A high CEACAM5 expression on tumor cells may be defined as being ≥2+ intensity in ≥50% of cancer cells, as measured by immunohistochemistry (IHC).

In certain embodiments, for patients with a body surface area (BSA) >2.2 m², the dose of the antibody-drug conjugate comprising an anti-CEACAM5-antibody may be calculated based on a BSA of 2.2 m².

### Pharmaceutical Compositions or Combinations

In some embodiments, pharmaceutical compositions or combinations of the present disclosure are such that the antibody-drug conjugate comprising an anti-CEACAM5-antibody is administered at a dose of from about 60 to about 210 mg/m², or from about 80 to about 170 mg/m², or from about 100 to about 170 mg/m² or from about 100 to about 150 mg/m². The anti-VEGFR-2 antibody is administered at a dose of from about 2 to about 20 mg/kg, or from about 4 to about 15 mg/kg, or from about 6 to about 10 mg/kg, or at about 8 mg/kg, or at about 10 mg/kg.

The pharmaceutical compositions or combinations of the present disclosure may be such that the antibody-drug conjugate comprising an anti-CEACAM5-antibody may be administered at a dose as above indicated.

In some embodiments, the pharmaceutical compositions or combinations of the present disclosure may be such that the antibody-drug conjugate comprising an anti-CEACAM5-antibody is administered at a dose of from about 60 to about 210 mg/m², or from about 80 to about 170 mg/m², or from about 100 to about 150 mg/m².

In various embodiments, the pharmaceutical compositions or combinations of the present disclosure may be such that the antibody-drug conjugate comprising the anti-CEACAM5 antibody is administered at a dose of about 60, 80, 70, 90, 100, 110, 120, 130, 135, 140, 145, 150, 155, 160, 165, 170, 175, 180, 185, 190, 195, 200, 205, or about 210 mg/m².

In various embodiments, the pharmaceutical compositions or combinations of the present disclosure may be such that the antibody-drug conjugate comprising the anti-CEACAM5 antibody is administered at a dose of about 60, 80, 100, 120, 135, 150, 170, 180, 190 or about 210 mg/m².

According to an embodiment, the pharmaceutical compositions or combinations of the present disclosure may be such that the antibody-drug conjugate comprising the anti-CEACAM5 antibody is administered at a dose of about 80 mg/m².

According to an embodiment, the pharmaceutical compositions or combinations of the present disclosure may be such that the antibody-drug conjugate comprising the anti-CEACAM5 antibody is administered at a dose of about 100 mg/m².

According to an embodiment, the pharmaceutical compositions or combinations of the present disclosure may be such that the antibody-drug conjugate comprising the anti-CEACAM5 antibody is administered at a dose of about 120 mg/m².

According to an embodiment, the pharmaceutical compositions or combinations of the present disclosure may be such that the antibody-drug conjugate comprising the anti-CEACAM5 antibody is administered at a dose of about 135 mg/m².

According to an embodiment, the pharmaceutical compositions or combinations of the present disclosure may be such that the antibody-drug conjugate comprising the anti-CEACAM5 antibody is administered at a dose of about 150 mg/m².

According to an embodiment, the pharmaceutical compositions or combinations of the present disclosure may be such that the antibody-drug conjugate comprising the anti-CEACAM5 antibody is administered at a dose of about 170 mg/m².

According to an embodiment, the pharmaceutical compositions or combinations of the present disclosure may be such that the antibody-drug conjugate comprising the anti-CEACAM5 antibody is administered at a dose of about 80, 100, 120, 135, 150 or 170 mg/m², as a loading dose.

According to an embodiment, the pharmaceutical compositions or combinations of the present disclosure may be such that the antibody-drug conjugate comprising the anti-CEACAM5 antibody is administered at a dose of about 120, 135, 150 or 170 mg/m², as a loading dose.

According to an embodiment, the pharmaceutical compositions or combinations of the present disclosure may be such that the antibody-drug conjugate comprising the anti-CEACAM5 antibody is administered at a dose of about 80 mg/m², as a loading dose.

According to an embodiment, the pharmaceutical compositions or combinations of the present disclosure may be such that the antibody-drug conjugate comprising the anti-CEACAM5 antibody is administered at a dose of about 100 mg/m², as a loading dose.

According to an embodiment, the pharmaceutical compositions or combinations of the present disclosure may be such that the antibody-drug conjugate comprising the anti-CEACAM5 antibody is administered at a dose of about 120 mg/m², as a loading dose.

According to an embodiment, the pharmaceutical compositions or combinations of the present disclosure may be such that the antibody-drug conjugate comprising the anti-CEACAM5 antibody is administered at a dose of about 135 mg/m², as a loading dose.

According to an embodiment, the pharmaceutical compositions or combinations of the present disclosure may be such that the antibody-drug conjugate comprising the anti-CEACAM5 antibody is administered at a dose of about 150 mg/m², as a loading dose.

According to an embodiment, the pharmaceutical compositions or combinations of the present disclosure may be such that the antibody-drug conjugate comprising the anti-CEACAM5 antibody is administered at a dose of about 170 mg/m², as a loading dose.

According to an embodiment, the pharmaceutical compositions or combinations of the present disclosure may be such that the antibody-drug conjugate comprising the anti-CEACAM5 antibody is administered at a dose of about 80, 100, 120, 135,150 or 170 mg/m², as a loading dose, on a first cycle of treatment, and then at a dose of about 80, 100, 120, 135, 150 or 170 mg/m², as a subsequent dose, on additional cycle(s).

According to an embodiment, the pharmaceutical compositions or combinations of the present disclosure may be such that the antibody-drug conjugate comprising the anti-CEACAM5 antibody is administered at a dose of about 80 mg/m², as a subsequent dose.

According to an embodiment, the pharmaceutical compositions or combinations of the present disclosure may be such that the antibody-drug conjugate comprising the anti-CEACAM5 antibody is administered at a dose of about 100 mg/m², as a subsequent dose.

According to an embodiment, the pharmaceutical compositions or combinations of the present disclosure may be such that the antibody-drug conjugate comprising the anti-CEACAM5 antibody is administered at a dose of about 120 mg/m², as a subsequent dose.

According to an embodiment, the pharmaceutical compositions or combinations of the present disclosure may be such that the antibody-drug conjugate comprising the anti-CEACAM5 antibody is administered at a dose of about 135 mg/m², as a subsequent dose.

According to an embodiment, the pharmaceutical compositions or combinations of the present disclosure may be such that the antibody-drug conjugate comprising the anti-CEACAM5 antibody is administered at a dose of about 150 mg/m², as a subsequent dose.

According to an embodiment, the pharmaceutical compositions or combinations of the present disclosure may be such that the antibody-drug conjugate comprising the anti-CEACAM5 antibody is administered at a dose of about 170 mg/m², as a subsequent dose.

According to an embodiment, the pharmaceutical compositions or combinations of the present disclosure may be such that the antibody-drug conjugate comprising the anti-CEACAM5 antibody is administered at a dose level of 80 mg/m², on a first cycle of treatment, and the cycle is about 2 weeks.

According to an embodiment, the pharmaceutical compositions or combinations of the present disclosure may be such that the antibody-drug conjugate comprising the anti-CEACAM5 antibody is administered at a dose of 80 mg/m², on a first cycle of treatment, as a loading dose, and at a dose of 80 mg/m², as a subsequent dose, on additional cycle(s). The cycle(s) may be about 2 weeks.

According to an embodiment, the pharmaceutical compositions or combinations of the present disclosure may be such that the antibody-drug conjugate comprising the anti-CEACAM5 antibody is administered at a dose level of 100 mg/m², on a first cycle of treatment, and the cycle is about 2 weeks.

According to an embodiment, the pharmaceutical compositions or combinations of the present disclosure may be such that the antibody-drug conjugate comprising the anti-CEACAM5 antibody is administered at a dose of 100 mg/m², on a first cycle of treatment, as a loading dose, and at a dose of 100 mg/m², as a subsequent dose, on additional cycle(s). The cycle(s) may be about 2 weeks.

According to an embodiment, the pharmaceutical compositions or combinations of the present disclosure may be such that the antibody-drug conjugate comprising the anti-CEACAM5 antibody is administered at a dose level of 150 mg/m², on a first cycle of treatment, and the cycle is about 2 weeks.

According to an embodiment, the pharmaceutical compositions or combinations of the present disclosure may be such that the antibody-drug conjugate comprising the anti-CEACAM5 antibody is administered at a dose of 150 mg/m², on a first cycle of treatment, as a loading dose, and at a dose of 80 mg/m², as a subsequent dose, on additional cycle(s). The cycle(s) may be about 2 weeks.

According to an embodiment, the pharmaceutical compositions or combinations of the present disclosure may be such that the antibody-drug conjugate comprising the anti-CEACAM5 antibody is administered at a dose of 150 mg/m², on a first cycle of treatment, as a loading dose, and at a dose of 100 mg/m², as a subsequent dose, on additional cycle(s). The cycle(s) may be about 2 weeks.

According to an embodiment, the pharmaceutical compositions or combinations of the present disclosure may be such that the antibody-drug conjugate comprising the anti-CEACAM5 antibody is administered at a dose of 170 mg/m², on a first cycle of treatment, and the cycle is about 2 or 3 weeks.

According to an embodiment, the pharmaceutical compositions or combinations of the present disclosure may be such that the antibody-drug conjugate comprising the anti-CEACAM5 antibody is administered at dose of 170 mg/m², as a loading dose, on a first cycle of treatment, and at a dose of 80 mg/m², as a subsequent dose, on additional cycle(s). The cycle(s) may be about 2 or 3 weeks. The cycle(s) may be of about 2 weeks.

According to an embodiment, the pharmaceutical compositions or combinations of the present disclosure may be such that the antibody-drug conjugate comprising the anti-CEACAM5 antibody is administered at dose of 170 mg/m², as a loading dose, on a first cycle of treatment, and at a dose of 100 mg/m², as a subsequent dose, on additional cycle(s). The cycle(s) may be about 2 or 3 weeks. The cycle(s) may be of about 2 weeks.

According to an embodiment, the pharmaceutical compositions or combinations of the present disclosure may be such that the antibody-drug conjugate comprising the anti-CEACAM5 antibody is administered at a dose level of 80 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at a dose of 80 mg/m², on a first cycle of treatment, as a loading dose, and at a dose of 80 mg/m², as a subsequent dose, on additional cycle(s). The cycle(s) may be about 3 weeks.

According to an embodiment, the pharmaceutical compositions or combinations of the present disclosure may be such that the antibody-drug conjugate comprising the anti-CEACAM5 antibody is administered at a dose level of 100 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at a dose of 100 mg/m², on a first cycle of treatment, as a loading dose, and at a dose of 80 mg/m², as a subsequent dose, on additional cycle(s). The cycle(s) may be about 3 weeks.

According to an embodiment, the pharmaceutical compositions or combinations of the present disclosure may be such that the antibody-drug conjugate comprising the anti-CEACAM5 antibody is administered at a dose level of 120 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at dose of 120 mg/m², as a loading dose, on a first cycle of treatment, and at a dose of 120 mg/m², as a subsequent dose, on additional cycle(s). The cycle(s) may be of about 3 weeks.

According to an embodiment, the pharmaceutical compositions or combinations of the present disclosure may be such that the antibody-drug conjugate comprising the anti-CEACAM5 antibody is administered at a dose level of 135 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at dose of 135 mg/m², as a loading dose, on a first cycle of treatment, and at a dose of 135 mg/m², as a subsequent dose, on additional cycle(s). The cycle(s) may be of about 3 weeks.

According to an embodiment, the pharmaceutical compositions or combinations of the present disclosure may be such that the antibody-drug conjugate comprising the anti-CEACAM5 antibody is administered at a dose level of 150 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at dose of 150 mg/m², as a loading dose, on a first cycle of treatment, and at a dose of 150 mg/m², as a subsequent dose, on additional cycle(s). The cycle(s) may be of about 3 weeks.

According to an embodiment, the pharmaceutical compositions or combinations of the present disclosure may be such that the antibody-drug conjugate comprising the anti-CEACAM5 antibody is administered at a dose level of 170 mg/m², on a first cycle of treatment, and the cycle is about 3 weeks.

According to an embodiment, the pharmaceutical compositions or combinations of the present disclosure may be such that the antibody-drug conjugate comprising the anti-CEACAM5 antibody is administered at dose of 170 mg/m², as a loading dose, on a first cycle of treatment, and at a dose of 170 mg/m², as a subsequent dose, on additional cycle(s). The cycle(s) may be of about 3 weeks.

According to an embodiment, the pharmaceutical compositions or combinations of the present disclosure may be such that the antibody-drug conjugate comprising the anti-CEACAM5 antibody is administered at a dose of 100 mg/m² on all cycles, i.e., on a first cycle of treatment and on additional cycle(s). The cycle(s) may be about 2 or 3 weeks.

In some embodiments, the pharmaceutical compositions or combinations of the present disclosure may be such that the anti-VEGFR-2 antibody is administered at a dose of from about 2 to about 20 mg/kg, or from about 4 to about 15 mg/kg, or from about 6 to about 10 mg/kg, or at about 8 mg/kg. The anti-VEGFR-2 antibody may be administered at a dose of about 2, 4, 6, 8, 10, 12, 14, 16, 18 or about 20 mg/kg.

In an embodiment, the pharmaceutical compositions or combinations of the present disclosure may be such that the anti-VEGFR-2 antibody is administered at 8 mg/kg.

In an embodiment, the pharmaceutical compositions or combinations of the present disclosure may be such that the anti-VEGFR-2 antibody is administered at 10 mg/kg.

In an embodiment, the anti-VEGFR-2 antibody is administered at 8 mg/kg and the cycle is about 2 weeks.

In an embodiment, the pharmaceutical compositions or combinations of the present disclosure may be such that the anti-VEGFR-2 antibody is administered at 10 mg/kg and the cycle is about 3 weeks.

The pharmaceutical compositions or combinations of the present disclosure may be such that the administered doses of the anti-VEGFR-2 antibody may be the same for a first cycle of treatment and the additional cycle(s).

The pharmaceutical compositions or combinations of the present disclosure may be such that the administered doses of the antibody-drug conjugate comprising an anti-CEACAM5-antibody may be of about 80 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks. The antibody-drug conjugate may be administered after the anti-VEGFR-2 antibody antibody-drug conjugate.

The pharmaceutical compositions or combinations of the present disclosure may be such that the administered doses of the antibody-drug conjugate comprising an anti-CEACAM5-antibody may be of about 80 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks. The antibody-drug conjugate may be administered after the anti-VEGFR-2 antibody antibody-drug conjugate.

The pharmaceutical compositions or combinations of the present disclosure may be such that the administered doses of the antibody-drug conjugate comprising an anti-CEACAM5-antibody may be of about 100 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks. The antibody-drug conjugate may be administered after the anti-VEGFR-2 antibody antibody-drug conjugate.

The pharmaceutical compositions or combinations of the present disclosure may be such that the administered doses of the antibody-drug conjugate comprising an anti-CEACAM5-antibody may be of about 100 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

The pharmaceutical compositions or combinations of the present disclosure may be such that the administered doses of the antibody-drug conjugate comprising an anti-CEACAM5-antibody may be of about 120 mg/m², at day 1 on a first cycle of treatment, as a loading dose, and at a dose of about 120 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

The pharmaceutical compositions or combinations of the present disclosure may be such that the administered doses of the antibody-drug conjugate comprising an anti-CEACAM5-antibody may be of about 135 mg/m², at day 1 on a first cycle of treatment, as a loading dose, and at a dose of about 135 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

The pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

The pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

The pharmaceutical compositions or combinations of the present disclosure may be such that the administered doses of the antibody-drug conjugate comprising an anti-CEACAM5-antibody may be of about 150 mg/m², at day 1 on a first cycle of treatment, as a loading dose, and at a dose of about 150 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

The pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 80 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

The pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 100 mg/m², as a subsequent dose, at day 1 on additional cycle(s), and that the administered doses of the anti-VEGFR-2 antibody may be of about 8 mg/kg, as a loading dose, on day 1 of a first cycle of treatment, and of about 8 mg/kg as a subsequent dose, at day 1 on additional cycle(s). The cycle(s) may be about 2 weeks.

The pharmaceutical compositions or combinations of the present disclosure may be such that the administered doses of the antibody-drug conjugate comprising an anti-CEACAM5-antibody may be of about 170 mg/m², at day 1 on a first cycle of treatment, as a loading dose, and at a dose of about 170 mg/m², as a subsequent dose, at day 1 on additional cycle(s), and that the administered doses of the anti-VEGFR-2 antibody may be of about 10 mg/kg, as a loading dose, on day 1 of a first cycle of treatment, and of about 10 mg/kg as a subsequent dose, at day 1 on additional cycle(s). The cycle(s) may be about 3 weeks.

In some embodiments, the antibody-drug conjugate comprising an anti-CEACAM5-antibody may be administered for a period of time ranging from about 30 minutes to about 3 hours, or from about 45 minutes to about 2.5 hours, or from about 1 hour to about 2 hours, or for about 1.5 hours. In some embodiments, the period of time may be of about 1.5 hours.

In some embodiments, the anti-VEGFR-2 antibody may be administered for a period of time ranging from about 20 minutes to about 2.5 hours, or from about 30 minutes to about 2 hours, or from about 45 minutes to about 1.5 hours, or for about 1 hour. In some embodiments, the period of time may be of about 1 hour.

The period of time between an administration of the anti-VEGFR-2 antibody and an administration of the antibody-drug conjugate comprising an anti-CEACAM5-antibody may range from about 20 minutes to about 5 hours, from about 30 minutes to about 3 hours, from about 40 minutes to about 2 hours, from about 50 minutes to about 1.5 hours, or may last about 1 hour.

In some embodiments, the period of time between an administration of the anti-VEGFR-2 antibody and an administration of the antibody-drug conjugate comprising an anti-CEACAM5-antibody may be about 1 hour.

In some embodiments, the anti-VEGFR-2 antibody is administered before the antibody-drug conjugate comprising an anti-CEACAM5-antibody.

In a further embodiment, the antibody-drug conjugate comprising an anti-CEACAM5-antibody, and an anti-VEGFR-2 antibody may be administered for a treatment comprising from about 8 to about 16 cycles. According to an embodiment, the cycle may be selected from a 1-week cycle, a 2-weeks cycle, a 3-weeks cycle, a 4-weeks cycle, a 5-weeks cycle, a 6-weeks cycle, or more weeks cycle.

In a further embodiment, the antibody-drug conjugate comprising an anti-CEACAM5-antibody, and an anti-VEGFR-2 antibody may be administered in a first cycle and at least in one additional cycle. Uses as disclosed herein may comprise from 2 to 16 cycles.

In some embodiments, a cycle (first or additional) may be about 2 weeks.

In some embodiments, a cycle (first or additional) may be about 3 weeks.

According to an embodiment, one cycle may comprise:
administering an anti-VEGFR-2 antibody at a dose of from 2 to 20 mg/kg, at least once in the cycle. and
administering the antibody-drug conjugate at a dose of from 60 to 210 mg/m², at least once in the cycle.

In one embodiment, the antibody-drug conjugate comprising an anti-CEACAM5-antibody is administered at a dose from 60 to 210 mg/m² on day 1 of the cycle.

In one embodiment, the anti-VEGFR-2 antibody is administered at a dose of from 2 to 20 mg/kg on day 1 of the cycle.

In one embodiment, the anti-VEGFR-2 antibody is administered at a dose of from 2 to 20 mg/kg on day 2 and day 5 of the cycle.

In one embodiment, the anti-VEGFR-2 antibody is administered at a dose of from 2 to 20 mg/kg on day 2 of the cycle.

In one embodiment, the antibody-drug conjugate comprising an anti-CEACAM5-antibody is administered at a dose of about 60, 80, 70, 90, 100, 110, 120, 130, 135, 140, 145, 150, 155, 160, 165, 170, 175, 180, 185, 190, 195, 200, 205, or about 210 mg/m² on day 1 of the cycle.

In one embodiment, the anti-VEGFR-2 antibody is administered at a dose of about 2, 4, 6, 8, 10, 12, 14, 16, 18 or about 20 mg/kg on day 1 of the cycle.

In one embodiment, the antibody-drug conjugate comprising an anti-CEACAM5-antibody is administered at a dose of 80, 100, 120, 135, 150 or 170 mg/m², as a loading dose, on day 1 of a first cycle of treatment, and at dose of 100 or 170 mg/m², as a subsequent dose, on day 1 of additional cycle(s). The anti-VEGFR-2 antibody is administered at a dose of about 8 or 10 mg/kg on day 1 of a first cycle of treatment and of additional cycle(s).

In one embodiment, the antibody-drug conjugate comprising an anti-CEACAM5-antibody is administered at a dose of 150 or 170 mg/m², as a loading dose, on day 1 of a first cycle of treatment, and at dose of 80 or 100 mg/m², as a subsequent dose, on day 1 of additional cycle(s). The anti-VEGFR-2 antibody is administered at a dose of about 8 mg/kg on day 1 of a first cycle of treatment and of additional cycle(s). A cycle may last 2 weeks.

In one embodiment, the antibody-drug conjugate comprising an anti-CEACAM5-antibody is administered at a dose of 100 or 170 mg/m², as a loading dose, on day 1 of a first cycle of treatment, and at dose of 100 or 170 mg/m², as a subsequent dose, on day 1 of additional cycle(s). The anti-VEGFR-2 antibody is administered at a dose of about 8 or 10 mg/kg on day 1 of a first cycle of treatment and of additional cycle(s). A cycle may last 2 or 3 weeks.

In one embodiment, the antibody-drug conjugate comprising an anti-CEACAM5-antibody is administered at a dose of 80 or 100 mg/m², on day 1 of a first cycle of treatment, and at dose of 80 or 100 mg/m², on day 1 of additional cycle(s). The anti-VEGFR-2 antibody is administered at a dose of about 8 mg/kg on day 1 of a first cycle of treatment and of additional cycle(s). A cycle may last 2 weeks.

In one embodiment, the antibody-drug conjugate is administered at a dose of 170 mg/m², on day 1 of a first cycle of treatment, and at dose of 100 or 170 mg/m², on day 1 of additional cycle(s). The anti-VEGFR-2 antibody is administered at a dose of about 8 or 10 mg/kg on day 1 of a first cycle of treatment and of additional cycle(s). A cycle may last 2 or 3 weeks.

In some embodiments, the antibody-drug conjugate comprising an anti-CEACAM5-antibody, and an anti-VEGFR-2 antibody may be administered once per cycle. The administration may be carried out on day one of each cycle.

According to some embodiments, in a use for treating a gastric cancer or a GEJ cancer one cycle (first and additional cycles) may comprise:
i) administering the antibody-drug conjugate comprising an anti-CEACAM5-antibody at a dose of from about 100 to about 170 mg/m², for example at a dose of about 150 mg/m² to about 170 mg/m², for example at about 135 mg/m², about 150 mg/m² or about 170 mg/m², once in the cycle, for example at day 1 of the cycle;
ii) administering an anti-VEGFR-2 antibody at a dose from about 8 mg/kg to about 10 mg/kg, once in the cycle, for example at day 1 of the cycle.

The administration may be carried out on day one of the cycle.

The cycle may be about two weeks.

The antibody-drug conjugate may be administered after the anti-VEGFR-2 antibody.

According to some embodiments, in a use for treating a gastric cancer or a GEJ cancer, a first cycle may comprise:
i) administering the antibody-drug conjugate at a dose of from about 150 to about 170 mg/m², for example at 170 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, a first cycle may comprise:
i) administering the antibody-drug conjugate comprising an anti-CEACAM5-antibody at a dose of from about 150 to about 170 mg/m², for example at 150 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 100 mg/m², once in the cycle, for example at day one of the cycle;
ii) administering an anti-VEGFR-2 antibody at a dose of about 8 mg/kg, once in the cycle, for example at day one of the cycle.

In such embodiment, such cycle may be an additional cycle.

In those embodiments, the antibody-drug conjugate may be administered after the anti-VEGFR-2 antibody.

A cycle may last 2 weeks.

According to some embodiments, in a use for treating a gastric cancer or GEJ cancer, a first cycle may comprise:
i) administering the antibody-drug conjugate comprising an anti-CEACAM5-antibody at a dose of about 150 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 80 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a gastric cancer or GEJ cancer, a first cycle may comprise:
i) administering the antibody-drug conjugate comprising an anti-CEACAM5-antibody at a dose of about 150 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 100 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a gastric cancer or GEJ cancer, a first cycle may comprise:
i) administering the antibody-drug conjugate comprising an anti-CEACAM5-antibody at a dose of about 170 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 80 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a gastric cancer or GEJ cancer, a first cycle may comprise:
i) administering the antibody-drug conjugate comprising an anti-CEACAM5-antibody at a dose of about 170 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 100 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a gastric cancer or GEJ cancer, a first cycle may comprise:
i) administering the antibody-drug conjugate comprising an anti-CEACAM5-antibody at a dose of about 100 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 100 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a gastric cancer or GEJ cancer, a first cycle may comprise:
i) administering the antibody-drug conjugate comprising an anti-CEACAM5-antibody at a dose of about 120 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 120 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a gastric cancer or GEJ cancer, a first cycle may comprise:
i) administering the antibody-drug conjugate comprising an anti-CEACAM5-antibody at a dose of about 135 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 135 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a gastric cancer or GEJ cancer, a first cycle may comprise:
i) administering the antibody-drug conjugate comprising an anti-CEACAM5-antibody at a dose of about 150 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 150 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a gastric cancer or GEJ cancer, a first cycle may comprise:
i) administering the antibody-drug conjugate comprising an anti-CEACAM5-antibody at a dose of about 170 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a gastric cancer or GEJ cancer, an additional cycle may comprise:
i) administering the antibody-drug conjugate comprising an anti-CEACAM5-antibody at a dose of about 170 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a lung cancer, such as a non-small cell lung cancer, such as non-squamous non-small-cell lung cancer (NSQ NSCLC), one cycle (first and additional cycles) may comprise:
i) administering the antibody-drug conjugate comprising an anti-CEACAM5-antibody at a dose of from about 80 to about 170 mg/m², for example at about 80 mg/m² or about 100 mg/m² or about 170 mg/m², once in the cycle, for example at day one of the cycle;
ii) administering an anti-VEGFR-2 antibody at a dose from about 8 to about 10 mg/kg, once in the cycle, for example at one of the cycle.

The administration may be carried out on day one of the cycle.

In such embodiment, the cycle is about two or three weeks.

The antibody-drug conjugate may be administered after the anti-VEGFR-2 antibody.

According to some embodiments, in a use for treating a lung cancer, such as a non-small cell lung cancer, such as non-squamous non-small-cell lung cancer (NSQ NSCLC), one cycle (first and additional cycles) may comprise:
i) administering the antibody-drug conjugate comprising an anti-CEACAM5-antibody at a dose of about 80 or about 100 mg/m², once in the cycle, for example at day 1 of the cycle;
ii) administering an anti-VEGFR-2 antibody at a dose of about 8 mg/kg, once in the cycle, for example at day 1 of the cycle.

The administration may be carried out on day one of the cycle.

In such embodiment, the cycle is about two weeks.

The antibody-drug conjugate may be administered after the anti-VEGFR-2 antibody.

According to some embodiments, in a use for treating a lung cancer, such as a non-small cell lung cancer, such as non-squamous non-small-cell lung cancer (NSQ NSCLC), one cycle (first and additional cycles) may comprise:
i) administering the antibody-drug conjugate comprising an anti-CEACAM5-antibody at a dose of from about 100 to about 170 mg/m², for example at about 170 mg/m², once in the cycle, for example at day of the cycle;
ii) administering an anti-VEGFR-2 antibody at a dose of about 8 or about 10 mg/kg, once in the cycle, for example at day one of the cycle.

The administration may be carried out on day one of the cycle.

In such embodiment, the cycle is about two or three weeks.

In those embodiments, the antibody-drug conjugate may be administered after the anti-VEGFR-2 antibody.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 80 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 80 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 100 mg/m² once in the cycle, for example at day one of the cycle;
ii) administering an anti-VEGFR-2 antibody at a dose of about 8 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 100 mg/m², once in the cycle, for example at day one of the cycle;
ii) administering an anti-VEGFR-2 antibody at a dose of about 8 mg/kg, once in the cycle, for example at day one of the cycle.

In those embodiments, the antibody-drug conjugate may be administered after the anti-VEGFR-2 antibody. A cycle may last 2 weeks.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 120 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 120 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 135 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 135 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 150 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 150 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

According to some embodiments, in a use for treating a lung cancer, such as a non-small cell lung cancer, such as non-squamous non-small-cell lung cancer (NSQ NSCLC), a first cycle may comprise:
i) administering the antibody-drug conjugate comprising an anti-CEACAM5-antibody at a dose of about 170 mg/m² once in the cycle, for example at day one of the cycle;
ii) administering an anti-VEGFR-2 antibody at a dose of about 10 mg/kg, once in the cycle, for example at day one of the cycle.

In such embodiment, such cycle may be a first cycle.

According to some embodiments, in a use for treating a lung cancer, such as a non-small cell lung cancer, such as non-squamous non-small-cell lung cancer (NSQ NSCLC), an additional cycle may comprise:
i) administering the antibody-drug conjugate comprising an anti-CEACAM5-antibody at a dose of about 170 mg/m², once in the cycle, for example at day one of the cycle;
ii) administering an anti-VEGFR-2 antibody at a dose of about 10 mg/kg, once in the cycle, for example at day one of the cycle.

In those embodiments, the antibody-drug conjugate may be administered after the anti-VEGFR-2 antibody. A cycle may last 3 weeks.

The unit “mg/m²” indicates the amount of compound in mg per m² of patient body surface administered per dose. The person skilled in the art is aware how to determine the required amount of compound for the patient to be treated based on his body surface, which in turn may be calculated based on height and body weight.

The unit “mg/kg” indicates the amount of compound in mg per kg of patient body administered per dose. The person skilled in the art is aware how to determine the required amount of compound for the patient to be treated based on his body weight.

In some embodiments, in the uses as disclosed herein, the administration of the antibody-drug conjugate comprising an anti-CEACAM5-antibody and/or the anti-VEGFR-2 antibody may be carried out by parenteral route. A suitable parenteral route may be intravenous infusion.

The present disclosure further relates to a pharmaceutical composition comprising an antibody-drug conjugate comprising an anti-CEACAM5-antibody, and further comprising an anti-VEGFR-2 antibody.

The present disclosure further relates to a pharmaceutical composition comprising an antibody-drug conjugate comprising an anti-CEACAM5-antibody, an anti-VEGFR-2 antibody, and at least one pharmaceutically acceptable excipient.

A pharmaceutical composition may comprise the antibody-drug conjugate comprising an anti-CEACAM5-antibody, and ramucirumab and a pharmaceutically acceptable excipient.

A pharmaceutical composition may comprise tusamitamab ravtansine, and ramucirumab and a pharmaceutically acceptable excipient.

According to some embodiments, the antibody-drug conjugate comprising an anti-CEACAM5-antibody and an anti-VEGFR-2 antibody may be formulated in the form of two separate pharmaceutical compositions, wherein (i) one pharmaceutical composition comprises the antibody-drug conjugate comprising an anti-CEACAM5-antibody, and (ii) the other pharmaceutical composition comprises the anti-VEGFR-2 antibody.

According to some embodiments, the antibody-drug conjugate comprising an anti-CEACAM5-antibody and an anti-VEGFR-2 antibody may be formulated in the form of two separate pharmaceutical compositions, wherein (i) one pharmaceutical composition comprises the antibody-drug conjugate comprising an anti-CEACAM5-antibody and at least one pharmaceutically acceptable excipient, and (ii) the other pharmaceutical composition comprises the anti-VEGFR-2 antibody and at least one pharmaceutically acceptable excipient.

The present disclosure further relates to a kit comprising (i) a pharmaceutical composition comprising the antibody-drug conjugate comprising an anti-CEACAM5-antibody and (ii) a pharmaceutical composition comprising an anti-VEGFR-2 antibody, in separate or combined formulations.

The present disclosure further relates to a kit comprising (i) a pharmaceutical composition comprising the antibody-drug conjugate comprising an anti-CEACAM5-antibody and at least one pharmaceutically acceptable excipient, and (ii) a pharmaceutical composition comprising an anti-VEGFR-2 antibody and at least one pharmaceutically acceptable excipient, in separate or combined formulations.

The present disclosure further relates to a pharmaceutical composition comprising an antibody-drug conjugate comprising an anti-CEACAM5-antibody, and further comprising an anti-VEGFR-2 antibody for use of treating of cancer.

The present disclosure further relates to a pharmaceutical composition comprising an antibody-drug conjugate comprising an anti-CEACAM5-antibody, an anti-VEGFR-2 antibody and at least one pharmaceutically acceptable excipient, for use of treating of cancer.

The present disclosure further relates to a kit comprising (i) a pharmaceutical composition comprising the antibody-drug conjugate comprising an anti-CEACAM5-antibody and (ii) a pharmaceutical composition comprising an anti-VEGFR-2 antibody, in separate or combined formulations, for use for treating of cancer.

The present disclosure further relates to a kit comprising (i) a pharmaceutical composition comprising the antibody-drug conjugate comprising an anti-CEACAM5-antibody and at least one pharmaceutically acceptable excipient, and (ii) a pharmaceutical composition comprising an anti-VEGFR-2 antibody and at least one pharmaceutically acceptable excipient, in separate or combined formulations, for use for treating of cancer.

“Pharmaceutical excipient” or “pharmaceutically acceptable excipient” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “pharmaceutically-acceptable carriers or excipients” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, buffer phosphate, acetate, citrate, succinate; amino acids and derivates such as histidine, arginine, glycine, proline, glycylglycine; inorganic salts NaCl, calcium chloride; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the disclosure can be formulated for a topical, oral, parenteral, intranasal, intravenous, intramuscular, subcutaneous or intraocular administration and the like. In an embodiment, the pharmaceutical compositions and combinations of the disclosure are formulated for intravenous administration.

In particular, the pharmaceutical compositions contain vehicles or excipients, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and in particular as a function of the mode of used, of the relevant pathology, or alternatively of the desired duration of treatment.

To prepare pharmaceutical compositions, an effective amount of antibody-drug conjugate comprising an anti-CEACAM5-antibody and of an anti-VEGFR-2 antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and injectable with the appropriate device or system for delivery without degradation. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

The antibody-drug conjugate comprising an anti-CEACAM5-antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the subsequent of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the patient being treated. The person responsible for administration will, in any event, determine the appropriate dose for the individual patient.

The antibody-drug conjugate comprising an anti-CEACAM5-antibody formulated for parenteral administration, such as intravenous or intramuscular injection, other pharmaceutically acceptable forms include, e.g., tablets or other solids for oral administration; time release capsules; and any other form currently used.

In certain embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide co glycolide nanoparticles that meet these requirements are contemplated for use in the present disclosure, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

### BRIEF DESCRIPTION OF THE SEQUENCES

SEQ ID NO: 1-5 show the sequences CDR-H1, CDR-H2, CDR-H3, CDR-L1 and CDR-L3 of the anti-CEACAM5-antibody (huMAb2-3).

SEQ ID NO: 6 shows the sequence of the variable domain of the heavy chain (VH) of the anti-CEACAM5-antibody (huMAb2-3).

SEQ ID NO: 7 shows the sequence of the variable domain of the light chain (VL) of the anti-CEACAM5-antibody (huMAb2-3).

SEQ ID NO: 8 shows the heavy chain sequence of the anti-CEACAM5-antibody (huMAb2-3).

SEQ ID NO: 9 shows the light chain sequence of the anti-CEACAM5-antibody (huMAb2-3).

SEQ ID NO: 10 shows the heavy chain sequence of the anti-VEGFR-2 antibody Ramucirumab.

SEQ ID NO: 11 shows the light chain sequence of the anti-VEGFR-2 antibody ramucirumab.

SEQ ID NO: 12-17 show the sequences CDR-H1, CDR-H2, CDR-H3, CDR-L1, CDR-L2 and CDR-L3 of the anti-VEGFR-2 antibody ramucirumab.

### BRIEF DESCRIPTION OF THE FIGURES

FIG. 1: Activity of the antibody-drug conjugate huMAb2-3-SPDB-DM4 and the anti-muVEGFR-2 antibody, DC-101, as single agents or in combination against subcutaneous gastric patient-derived xenograft, STO-IND-0006, in SCID mice. Tumor volume evolution by treatment group. The curves represent medians + or − MAD at each day for each group.

FIG. 2: Activity of the antibody-drug conjugate huMAb2-3-SPDB-DM4 and the anti-muVEGFR-2 antibody, DC-101, as single agents or in combination against subcutaneous gastric patient-derived xenograft, SA-STO-0014, in SCID mice. Tumor volume evolution by treatment group. The curves represent medians + or − MAD at each day for each group.

### EXAMPLES

### Example 1: Activity of the Antibody-Drug Conjugate huMAb2-3-SPDB-DM4 in Combination with the Anti-muVEGFR-2 Antibody, DC-101, Against a Subcutaneous Gastric Patient-Derived Xenograft, STO-IND-0006, in SCID Mice

DC-101 is a rat anti-mouse VEGFR-2 mAb frequently used as a surrogate mAb for ramucirumab for in vivo studies, because ramucirumab does not cross react with mouse VEGFR-2.

### Experimental Procedure

The activity of huMAb2-3-SPDB-DM4 or anti-muVEGFR-2, DC-101, was evaluated as single agent or in combination in a subcutaneous gastric patient-derived xenografts (PDX), STO-IND-0006, implanted s.c. in female SCID mice. Control groups were left untreated. The doses of the compounds used are given in mg/kg.

For STO-IND-0006 PDX, mice were randomized in 4 groups (n=10 to 12) on day 26 post tumour implantation when median tumour burden reached 172.5 mm³. huMAb2-3-SPDB-DM4 was administered at 5 mg/kg following 3 weekly cycles of IV administrations on days 24, 31 and 38 and the DC-101 antibody was administered at 20 mg/kg following 3 weekly cycles of IV administrations on days 25, 28, 32, 35, 39 and 42.

For the evaluation of anti-tumor activity, animals were weighed daily and tumors were measured 2 times weekly by caliper. A dosage producing a 20% weight loss at nadir (mean of group) or 10% or more drug deaths, was considered an excessively toxic dosage. Animal body weights included the tumor weights. Tumor volume were calculated using the formula mass (mm³)=[length (mm)×width (mm)×width (mm)]/2. The primary efficacy end points are ΔT/ΔC, percent median regression, partial and complete regressions (PR and CR).

Changes in tumor volume for each treated (T) and control (C) are calculated for each tumor by subtracting the tumor volume on the day of first treatment (staging day) from the tumor volume on the specified observation day. The median ΔT is calculated for the treated group and the median ΔC is calculated for the control group. Then the ratio ΔT/ΔC is calculated and expressed as a percentage: ΔT/ΔC=(delta T/delta C)×100.

The dose is considered as therapeutically active when ΔT/ΔC is lower than 40% and very active when ΔT/ΔC is lower than 10%. If ΔT/ΔC is lower than 0, the dose is considered as highly active and the percentage of regression is dated (Plowman J, Dykes D J, Hollingshead M, Simpson-Herren L and Alley M C. Human tumor xenograft models in NCI drug development. In: Feibig H H BA, editor. Basel: Karger.; 1999 p 101-125):

% tumor regression is defined as the % of tumor volume decrease in the treated group at a specified observation day compared to its volume on the first day of first treatment.

At a specific time point and for each animal, % regression is calculated. The median % regression is then calculated for the group:

$$\% regression ( at t ) = \frac{{volume}_{t 0} - {volume}_{t}}{{volume}_{t 0}} \times 100$$

Partial regression (PR): Regressions are defined as partial if the tumor volume decreases to 50% of the tumor volume at the start of treatment.

Complete regression (CR): Complete regression is achieved when tumor volume=0 mm³ (CR is considered when tumor volume cannot be recorded).

### Results

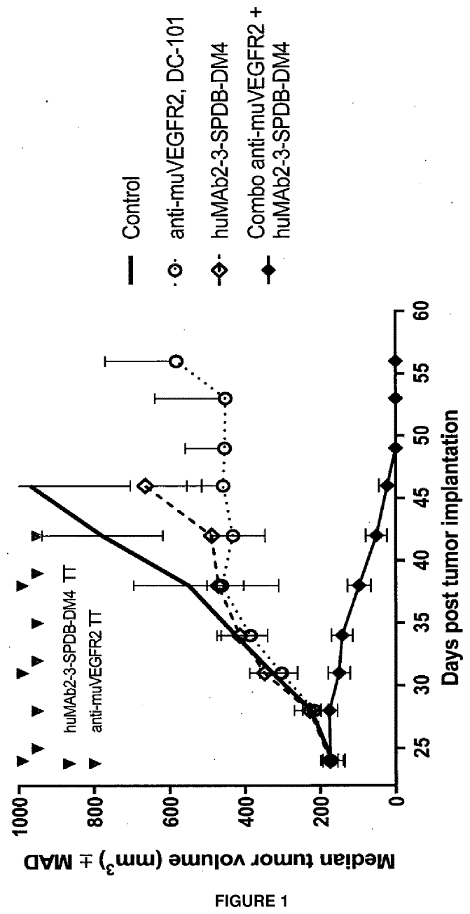

The results for experiment in STO-IND-0006 PDX are presented on FIG. 1 and Table 1.

The STO-IND-0006 PDX is an aggressive tumor, it can be cachexic and induces body weight loss and requires premature ethical euthanasia of one mouse before study end in both control and huMAb2-3-SPDB-DM4 groups. huMAb2-3-SPDB-DM4 and DC-101 were administered at doses lower than maximal tolerated dose (MTD) and treatments were well tolerated and did not induce additional toxicity.

The huMAb2-3-SPDB-DM4 as a single agent was inactive with a ΔT/ΔC on D46 equal to 60%. The DC-101 as single agent was active with a ΔT/ΔC equal to 33% (p=0.0050 vs control).

The combination between huMAb2-3-SPDB-DM4 and DC-101 was highly active with a ΔT/ΔC inferior to 0% (p<0.0001 vs control), a tumor regression of 100%, 9/9 PR and 6/9 CR. The effect of the combination of huMAb2-3-SPDB-DM4 with DC-101 was significantly different from the effect of huMAb2-3-SPDB-DM4 alone from day 31 to day 46 (study end of huMAb2-3-SPDB-DM4 group) and significantly different from the effect of DC-101 alone from day 31 to day 56.

In conclusion to the experiment in the STO-IND-0006 PDX, the combination of huMAb2-3-SPDB-DM4 and DC-101 after 3 cycles of treatment is highly active inducing complete regression in despite of the lack of activity of huMAb2-3-SPDB-DM4 as single agent and of a moderate activity of DC-101 as single agent.

TABLE 1
Activity of huMAb2-3-SPDB-DM4 and anti-muVEGFR2, DC-101, in combination against
subcutaneous gastric Patient-Derived-Xenograft, STO-IND-0006 in SCID mice

| | | | | | Mean body | Median | Median % | | | | |
|---|---|---|---|---|---|---|---|---|---|---|---|
| | | Dosage | | Drug | weight | ΔT/ΔC | of | | | Biosatitic | |
| | | in mg/kg | Schedule | death | change in % | in % | regression | Regression | | p value^{a} | Biological |
| Agent | Route | (total) | in day | (day) | at nadir (day) | (D 46) | (day) | PR | CR | (D 46) | comments |
| huMAb2- | IV | 5 (15) | 24, 34, 38 | 0/10 | −7.2 (42) | 60 | — | 0/10 | 0/10 | ns | Inactive |
| 3-SPDB- | | | | | | | | | | | |
| DM4 | | | | | | | | | | | |
| DC-101 | IV | 20 (80) | 25, 28, 32, | 0/10 | −2.1 (25) | 33 | — | 0/10 | 0/10 | 0.0050 | Active |
| | | | 35, 39, 42 | | | | | | | | |
| huMAb2- | IV | 5 (15) | 24, 34, 38 | 0/10* | −3.3 (32) | <0 | 100 (D 49) | 3/9* | 0/9* | <0.0001 | Highly |
| 3-SPDB- | IV | 20 (80) | 25, 28, 32, | | | | | | | | active |
| DM4 | | | 35, 39, 42 | | | | | | | | |
| DC-101 | | | | | | | | | | | |
| Control | — | — | — | — | −7.5 (46) | — | — | — | — | — | — |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression
*one mouse dead by accident during injection (bubble presence) and was excluded from analysis

### Example 2: Activity of the Antibody-Drug Conjugate huMAb2-3-SPDB-DM4 in Combination with the Anti-muVEGFR-2 Antibody, DC-101, Against a Subcutaneous Gastric Patient-Derived Xenograft, SA-STO-0014, in SCID Mice

### Experimental Procedure

The activity of huMAb2-3-SPDB-DM4 and DC-101 was evaluated as single agent or in combination in a subcutaneous gastric PDX, SA-STO-0014, implanted s.c. in female SCID mice. Control groups were left untreated. The doses of the compounds used are given in mg/kg.

For SA-STO-0014 PDX, mice were randomized in 4 groups (n=10 to 12) on day 26 post tumour implantation when median tumour burden reached 161.5 mm³. huMAb2-3-SPDB-DM4 was administered at 5 mg/kg following 2 weekly cycles of IV administrations on days 21 and 28. The DC-101 antibody was administered at 20 mg/kg following 2 weekly cycles of IV administrations on days 22, 25, 29 and 32.

See above (Ex 1) for the conditions of anti-tumor activity and toxicity evaluation.

### Results

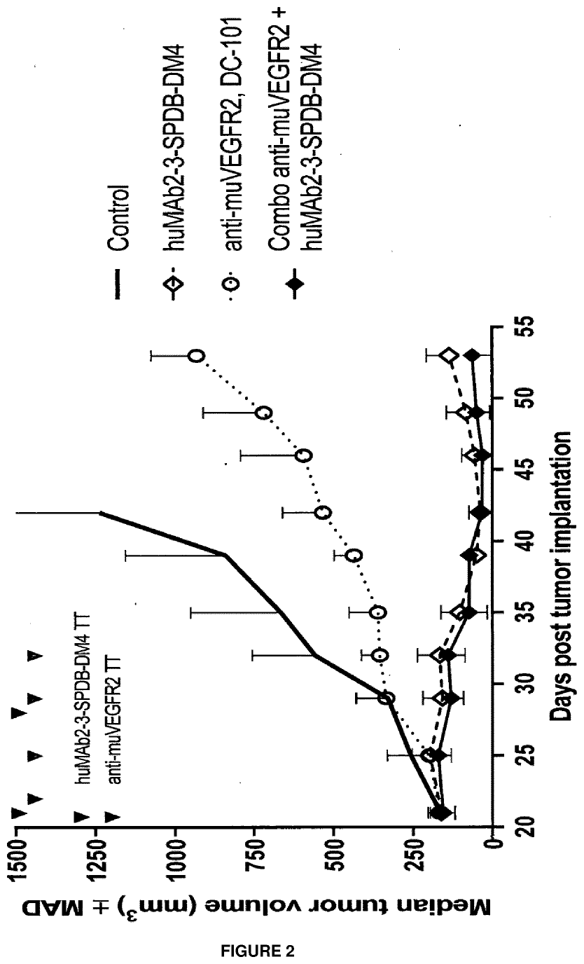

The results for the SA-STO-0014 PDX are presented on FIG. 2 and Table 2

huMAb2-3-SPDB-DM4 and DC-101 treatments were well tolerated and did not induce toxicity.

The huMAb2-3-SPDB-DM4 as a single agent was highly active with a ΔT/ΔC on D42 inferior to 0% (p<0.0001 vs control), a tumor regression of 75%, 7/10 PR and 3/10 CR. The DC-101 as single agent was active with a ΔT/ΔC equal to 36% (p<0.0001 vs control).

The combination between huMAb2-3-SPDB-DM4 and DC-101 was highly active with a ΔT/ΔC inferior to 0% (p<0.0001 vs control), a tumor regression of 83%, 9/10 PR and 6/10 CR. The effect of the combination of huMAb2-3-SPDB-DM4 with DC-101 was significantly different from the effect of huMAb2-3-SPDB-DM4 alone on day 53 and significantly different from the effect of DC-101 alone from day 29 to day 53.

In conclusion to the experiment in SA-STO-0014 PDX, huMAb2-3-SPDB-DM4 was highly active and the combination of huMAb2-3-SPDB-DM4 and DC-101 after 2 cycles of treatment allowed to maintain a longer high activity compared to huMAb2-3-SPDB-DM4 as single agent.

TABLE 2
Activity of huMAb2-3-SPDB-DM4 and anti-muVEGFR2, DC-101, in combination against
subcutaneous gastric Patient-Derived-Xenograft, SA-STO-0014 in SCID mice

| | | | | | Mean body | Median | Median % | | | | |
|---|---|---|---|---|---|---|---|---|---|---|---|
| | | Dosage | | Drug | weight | ΔT/ΔC | of | | | Biosatitic | |
| | | in mg/kg | Schedule | death | change in % | in % | regression | Regression | | p value^{a} | Biological |
| Agent | Route | (total) | in day | (day) | at nadir (day) | (D 42) | (day) | PR | CR | (D 45) | comments |
| huMAb2- | IV | 5 (10) | 21, 28 | 0/10 | −1.4 (22) | <0 | 75 | 7/10 | 3/10 | <0.0001 | Highly |
| 3-SPDB- | | | | | | | | | | | active |
| DM4 | | | | | | | | | | | |
| DC-101 | IV | 20 (80) | 22, 25, 29, | 0/10 | −1.2 (22) | 36 | — | 0/10 | 0/10 | 0.0158 | Active |
| | | | 32 | | | | | | | | |
| huMAb2- | IV | 5 (10) | 21,28 | 0/10 | −2.7 (29) | <0 | 83 | 9/10 | 6/10 | <0.0001 | Highly |
| 3-SPDB- | IV | 20 (80) | 22, 25, 29, | | | | | | | | active |
| DM4 | | | 32 | | | | | | | | |
| DC-101 | | | | | | | | | | | |
| Control | — | — | — | — | 0.0 (22) | — | — | — | — | — | — |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression

### Example 3: Efficacy in Combination with Anti-Vascular Endothelial Growth Factor Receptor-2 (VEGFR-2) Antibody in Gastric Patient Derived Xenografts (PDX)

Cyramza® (ramucirumab) is an anti-VEGFR-2 antibody that was approved in combination with paclitaxel in second line gastric cancer. In vivo evaluation of tusamitamab ravtansine (ADC of the disclosure) in combination with ramucirumab was done against gastric PDX to provide convincing data to support the substitution of paclitaxel with tusamitamab ravtansine in combination with ramucirumab in clinic. Because ramucirumab is unable to bind to murine VEGFR-2, in vivo studies were conducted in patient derived xenografts (PDX) implanted mice using a surrogate antibody that recognizes the murine receptor (anti-muVEGFR-2). Administration of an anti-muVEGFR-2 antibody to these animals was able to inhibit the growth of an array of tumor types, including primary human gastric tumor fragments.

### Experimental Procedure

Carcinoembryonic antigen related cell adhesion molecule 5 expression is polarized (ie, restricted to the apical side of well differentiated cells) in the epithelial gastric patient-derived xenograft PDX STO-IND-0006. Antitumor activity of tusamitamab ravtansine was evaluated alone and in combination with anti muVEGFR-2 antibody to SCID mice bearing this PDX, in comparison with paclitaxel/anti muVEGFR-2 antibody combination after 2 weekly cycles.

Treatments were initiated on day 27 post tumor implantation. Tusamitamab ravtansine was administered IV at 5 mg/kg on days 27 and 34. Paclitaxel was administered IV at 20 mg/kg on

days 27 and 34. Anti-muVEGFR-2 antibody was administered IV at 20 mg/kg on days 28, 31, 35, and 38 (Table 10).

The primary efficacy endpoints were tumor volume changes from baseline summarized by the ratio of medians between treated and control groups (ΔT/ΔC) expressed in percentage, the percent median regression defined as the % of tumor volume decrease in the treated group at a specified observation day compared to its volume on the first day of treatment, partial regression (PR) if the tumor volume decreases to at least 50% of the tumor volume at the start of treatment and complete regression (CR) when tumor volume cannot be recorded (ie, is less than 14 mm³).

Interpretation of the ΔT/ΔC expressed in percentage was based on the following criteria: >40%: inactive; 540%: active; <10%: very active; <0%: highly active. Tumor-free survivor (TFS) is defined as the number of animals with undetectable tumors at the end of the study (ie, 120 days post tumor implantation).

### Results

STO-IND-0006 PDX was observed to be cachexic and induced body weight loss (BWL) even in the control, untreated tumor bearing mice. Paclitaxel was administered at maximum tolerated dose (MTD) determined in non-bearing tumor mice. In mice bearing STO-IND-0006 tumor, additive BWL was observed for paclitaxel alone or in combination that leads to individual drastic BWL (>20%) or death. Body weight loss was observed from day 48 for all groups treated by paclitaxel or in which no activity was observed (control and tusamitamab ravtansine treated groups). No BWL was observed for groups treated by anti muVEGFR-2 antibody and the combination of tusamitamab ravtansine with anti-muVEGFR-2 antibody.

As depicted in Table 10, tusamitamab ravtansine alone was inactive with a ΔT/ΔC equal to 91%. Paclitaxel alone was significantly active with a ΔT/ΔC equal to 28% (p=0.0011). Anti-muVEGFR-2 antibody alone was significantly active with a ΔT/ΔC equal to 29% (p=0.0011).

The combination of tusamitamab ravtansine and anti-muVEGFR-2 antibody was significantly highly active with a ΔT/ΔC inferior to 0% (p<0.0001), a tumor regression of 17% and 3 PR out of 9 mice, and was significantly more active than both single agents indicating a therapeutic synergy in this tusamitamab ravtansine non-sensitive PDX.

TABLE 10
Activity of tusamitamab ravtansine alone and in combination with
anti-VEGFR-2 antibody after 2 weekly cycles against the gastric
PDX STO-IND-0006 engrafted subcutaneously in SCID mice

| | Dosage in mg/kg | | | |
|---|---|---|---|---|
| Agent | (total) | | Schedule in day | Activity |
| Tusamitamab | 5 | (10) | 27, 31 | ΔT/ΔC = 91% Inactive |
| ravtansine | | | | |
| Paclitaxel | 20 | (40) | 27, 31 | ΔT/ΔC = 28% Active |
| Anti- | 20 | (80) | 28, 31, 35, 38 | ΔT/ΔC = 29% Active |
| muVEGFR-2 | | | | |
| Tusamitamab | 5 | (10) | 27, 31 | ΔT/ΔC < 0% Highly active |
| ravtansine & | | | | |
| anti-muVEGFR- | 20 | (80) | 28, 31, 35, 38 | R = 17% 3/9 PR |
| 2 | | | | |
| Paclitaxel & | 20 | (40) | 27, 31 | ΔT/ΔC < 0% Highly active |
| anti-muVEGFR- | | | | |
| 2 | 20 | (80) | 28, 31, 35, 38 | R = 24% 1/9 PR |

Abbreviations:
ΔT/ΔC = ratio of medians of tumor volume changes from baseline between treated and control groups;
PR: Partial tumor regression;
CR: Complete tumor regression,
R: Percent median regression

### Example 4: Gastric Patient-Derived Xenograft STO-IND-0006 after 3 Weekly Cycle

### Experimental Procedure

Another experiment was performed on the same patient derived xenografts (PDX) with a 3-week treatment period for the tusamitamab ravtansine and anti-muVEGFR-2 antibody combination. The combination with paclitaxel was not evaluated due to the paclitaxel toxicity observed in the 2 weeks treatment study.

Treatments were initiated on day 24 post tumor implantation. Tusamitamab ravtansine was administered IV at 5 mg/kg on days 24, 31, and 38. Anti-muVEGFR-2 antibody was administered IV at 20 mg/kg on days 25, 28, 32, 35, 39, and 42 (Table 11).

### Results

STO-IND-0006 PDX was cachexic and induced individual drastic body weight loss BWL (>20%) or death for group in which no activity was observed (control and tusamitamab ravtansine treated groups). No BWL was observed for groups treated by anti-muVEGFR-2 antibody and the combination of tusamitamab ravtansine with anti-muVEGFR-2 antibody.

As shown in table 11, tusamitamab ravtansine alone was inactive with a ΔT/ΔC equal to 60%. Anti-muVEGFR-2 antibody alone was significantly active with a ΔT/ΔC equal to 33% (p=0.0166).

The combination of tusamitamab ravtansine and anti-muVEGFR-2 antibody was significantly highly active with a ΔT/ΔC inferior to 0% (p<0.0001), a tumor regression of 100%, 9 PR out of 9 mice, and 6 CR out of 9 mice, and was significantly more active than both single agents indicating a therapeutic synergy in this tusamitamab ravtansine non-sensitive PDX.

TABLE 11
Activity of tusamitamab ravtansine alone and in combination with
anti-VEGFR-2 antibody after 3 weekly cycles against the gastric
PDX STO-IND-0006 engrafted subcutaneously in SCID mice

| | Dosage in mg/kg | | | |
|---|---|---|---|---|
| Agent | (total) | | Schedule in day | Activity |
| Tusamitamab | 5 | (15) | 24, 31, 38 | ΔT/ΔC = 60% Inactive |
| ravtansine | | | | |
| Anti- | 20 | (120) | 25, 28, 32, 35, 39, 42 | ΔT/ΔC = 33% Active |
| muVEGFR-2 | | | | |
| Tusamitamab | 5 | (15) | 24, 31, 38 | ΔT/ΔC < 0% Highly active |
| ravtansine & | | | | |
| anti-muVEGFR- | 20 | (120) | 25, 28, 32, 35, 39, 42 | R = 100% 9/9 PR, 6/9CR |
| 2 | | | | |

Abbreviations:
ΔT/ΔC = ratio of medians of tumor volume changes from baseline between treated and control groups;
PR: Partial tumor regression;
CR: Complete tumor regression,
R: Percent median regression

In conclusion, a synergy for combination of tusamitamab ravtansine with anti-muVEGFR-2 antibody was observed, both in paclitaxel and tusamitamab ravtansine non-sensitive PDX. An impressive synergy (complete regression) for combination of tusamitamab ravtansine with anti muVEGFR-2 antibody was observed in the 3 weeks treatment study in a PDX non sensitive to tusamitamab ravtansine alone. A more favorable safety profile was observed for the combination of tusamitamab ravtansine with anti-muVEGFR-2 antibody versus the combination of paclitaxel with anti-muVEGFR-2 antibody. These data support the substitution of paclitaxel to tusamitamab ravtansine in combination with ramucirumab for gastric indication in clinic.

### Example 5: Antibody-Drug Conjugate huMAb2-3-SPDB-DM4 in Combination with the Anti-VEGFR-2 Antibody Ramucirumab in Pretreated Participants with Advanced Gastric or Gastroesophageal Junction (GEJ) Adenocarcinoma with CEACAM5-Positive Tumors

### Rationale

Synergy of huMAb2-3-SPDB-DM4 activity in combination with ramucirumab may lead to improved efficacy in treating gastric cancer (GC) or gastroesophageal junction (GEJ) adenocarcinoma cancer patients with a high unmet need. Further this combination may have a better safety profile compared to the combination of paclitaxel with ramucirumab.

### Experimental Procedure

A single group, treatment, Phase 2, open-label, single-arm clinical study was conducted to confirm the recommended dose (RD), safety, pharmacokinetic (PK), and preliminary antitumor activity of huMAb2-3-SPDB-DM4 combined with the anti-VEGFR-2 antibody ramucirumab in participants previously treated for GC or GEJ with CEACAM5-positive (defined as CEACAM5 immunohistochemical [IHC] intensity ≥2+ in ≥50% of cells) tumors.

The participants' inclusion criteria were as follows:

### Age

At least 18 years of age (or the legal age of consent in the jurisdiction in which the study is taking place) at the time of signing the informed consent.

### Type of Participant and Disease Characteristics

Histologically or cytologically confirmed diagnosis of gastric or GEJ adenocarcinoma.

Metastatic disease or locally advanced, unresectable disease.

Measurable disease by RECIST 1.1, as determined by the Investigator.

At least 1 measurable lesion is required. A previously irradiated tumor lesion is considered measurable if progression has been demonstrated in the lesion. The lesion must be 10 mm in the longest diameter (except lymph nodes, which must have a short axis 15 mm) as imaged in computed tomography (CT; preferred) or magnetic resonance imaging (MRI) scans.

Documented disease progression during or after first-line therapy containing platinum and/or fluoropyrimidine agents, and if appropriate, HER2 therapy.

No more than 1 previous line of chemotherapy is allowed. Previous treatment with an immune checkpoint inhibitor is allowed. Adjuvant/neoadjuvant treatment for a participant who had disease progression during or within 6 months of completing platinum and/or fluoropyrimidine treatment will be considered as first-line treatment.

Expression of CEACAM5 as demonstrated prospectively by a centrally assessed IHC assay of 2+ in intensity involving at least 50% of the tumor cell population in an archival tumor sample (or, if not available, a fresh biopsy sample).

At least 5 fresh-cut slides of formalin-fixed, paraffin embedded (FFPE) tumor tissue sectioned at a thickness of 4 to 5 μm are required. If less material is available, the participant could still be considered eligible after discussion with the Sponsor, who may assess and confirm that the available material is sufficient for key evaluations.

Eastern Cooperative Oncology Group (ECOG) performance status 0-1.

### Sex, Contraceptive/Barrier Method and Pregnancy Testing Requirements

All participants (male and female)

Contraceptive use by men and women should be consistent with local regulations regarding the methods of contraception for those participating in clinical studies.

### a) Male Participants

Male participants are eligible to participate if they agree to the following during the intervention period and for at least 4 months after the last dose of study intervention:
Refrain from donating sperm
Plus either:
Be abstinent from heterosexual or homosexual intercourse as their preferred and usual lifestyle (abstinent on a long term and persistent basis) and agree to remain abstinent

OR
Must agree to use contraception/barrier as detailed below:

Agree to use male condom when engaging in any activity that allows for passage of ejaculate to another person

### b) Female Participants

A female participant is eligible to participate if she is not pregnant or breastfeeding, and at least 1 of the following conditions applies:
Is not a woman of childbearing potential (WOCBP)

OR
Is a WOCBP and agrees to use a contraceptive method that is highly effective (with a failure rate of <1% per year), preferably with low user dependency, during the intervention period and for at least 7 months after the last dose of study intervention and agrees not to donate eggs (ova, oocytes) for the purpose of reproduction during this period.

A WOCBP must have a negative highly sensitive pregnancy test (urine or serum as required by local regulations) before the first dose of study intervention. The Investigator is responsible for review of medical history, menstrual history, and recent sexual activity to decrease the risk for inclusion of a woman with an early undetected pregnancy.

### Informed Consent

Capable of giving signed informed consent.

In the pre-screening phase, patients with GC or GEJ had tumor tissue tested centrally to assess proportions of CEACAM5-positive cells and intensity of expression of CEACAM5 expression. For this analysis, at least 5 fresh-cut slides of formalin-fixed, paraffin embedded (FFPE) tumor tissue sectioned at a thickness of 4 to 5 μm were analyzed a standard immunohistochemistry protocol. In brief, CEACAM5 tumor expression was assessed at prescreening on the most recent available archival tumor sample (i.e., archive tumor tissue at diagnosis, archive tumor tissue at surgery, or tumor sample before inclusion in the study and not under anticancer treatment). The level and pattern of CEACAM5 expression in tumor tissues were determined using IHC with an anti-CEACAM5 antibody run on Dako/Agilent Autostainer Link 48 IHC platform. Interpretation of CEACAM5 reactivity was performed by a board-certified pathologist using semi-quantitative Percent Scores (calculated by summing the percentages of intensities ≥2+) or H-score for CEACAM5 plasma membrane staining (whole or polarized) in tumor cells. Cytoplasmic staining was also evaluated.

Only participants with positive results, defined as CEACAM5 expression of ≥2+ in intensity involving at least 50% of the tumor cell population, in tumor sample entered the screening phase.

This was a 2-part study.

In Part 1, participants received ramucirumab 8 mg/kg followed by huMAb2-3-SPDB-DM4 at 170 mg/m² at day 1 Cycle 1, or ramucirumab 8 mg/kg followed by huMAb2-3-SPDB-DM4 100 mg/m² at Cycle 2 and every two weeks (Q2W) in all subsequent (or additional) cycles (Table 3).

In case it was decided to reduce the initial loading dose of huMAb2-3-SPDB-DM4 to DL-1 (dose level −1), a huMAb2-3-SPDB-DM4 loading dose of 150 mg/m² was administered to participants on day 1 of Cycle 1.

The tolerability of the initial DL was assessed as follows: if ≥2 of the first 3 patients or of the 6 patients treated at the initial DL presented with DLTs, then it may be decided to decrease the dose of huMAb2-3-SPDB-DM4 to DL-1 (150 mg/m² in combination with 8 mg/kg ramucirumab). The tolerability of the reduced DL (DL-1) was assessed in at least 6 participants

TABLE 3
Dose levels for Part 1 (safety run-in)

| Dose level (DL) | huMAb2-3-SPDB-DM4 | Ramucirumab |
|---|---|---|
| Starting dose | 170 mg/m² Q2W Cycle 1; | 8 mg/kg Q2W |
| | 100 mg/m² Q2W Cycle 2 and thereafter | |
| Minus −1 (DL −1) | 150 mg/m² Q2W Cycle 1; | 8 mg/kg Q2W |
| | 100 mg/m² Q2W Cycle 2 and thereafter | |

BSA = body surface area; DL −1 = dose level −1; Q2W = every 2 weeks.

Infusion of huMAb2-3-SPDB-DM4 was administered at least 1 hour after the end of ramucirumab infusion for at least the first 2 cycles. For participants with a BSA >2.2 m², the huMAb2-3-SPDB-DM4 dose was calculated based on a BSA of 2.2 m².

In Part 2 of the study, the recommended dose (RD) confirmed in Part 1 was evaluated for activity in 26 additional participants. A total of 32 participants, including participants treated at the recommended dose in Part 1, are evaluated for activity.

The duration of the study for a participant included:
Screening period: up to 28 days.
Treatment period: once successfully screened, enrolled participants received study intervention until disease progression, unacceptable adverse event (AE), death, initiation of a new anticancer therapy, or the participant's or investigator's decision to stop the treatment. Each cycle of treatment had a duration of 2 weeks. After discontinuing study intervention, participants returned to the study site approximately 30 days after the last administration or before the participants received another anti-cancer therapy, whichever was earlier, for end-of-treatment assessments.
Safety follow-up visit was performed approximately 90 days after the last administered dose. If any ongoing related adverse event was resolved or stabilized, no further follow-up visit is needed.

The expected duration of study intervention for participants varied, based on the disease progression date; the median expected duration of the study per participant was estimated as 34 weeks (up to 4 weeks for screening, a median of 18 weeks for treatment, and a median of 12 weeks for end-of-treatment assessments and the safety follow-up visit).

Infusion of the drugs via a central line was preferred, if available. Prior to dosing, each participant's dose was individually prepared by the study pharmacist and labeled with protocol number, participant number, and treatment description.

### Investigational Medicinal Products (IMP)

Ramucirumab was administered after huMAb2-3-SPDB-DM4 170 or 150 mg//m² and prior administration of huMAb2-3-SPDB-DM4 100 mg/m².
Formulation: CYRAMZA® (ramucirumab) is a concentrate for solution for infusion supplied in 10 mL or 50 mL single-use vial. Each vial contained either 100 mg ramucirumab in 10 mL (10 mg/mL) or 500 mg ramucirumab in 50 mL (10 mg/mL).
Route of administration: intravenous (IV) infusion
Dose regimen: ramucirumab was administered as an 8 mg/kg IV infusion administered over 1 hour on day 1 of every 2-weeks cycle.

Using a controlled infusion pump, ramucirumab was administered by intravenous (IV) infusion over 1 hour on day 1 of each cycle. If the first infusion was tolerated, all subsequent ramucirumab infusions may be administered over 30 minutes. On day 1 of each treatment cycle, the patient's BSA was determined using the most recent weight available on the day of the infusion preparation: the weight on the day of the infusion or the most recent weight, assuming it was assessed in a reasonable time frame according to Investigator assessment. If the infusion was prepared with the most recent weight assessed in a reasonable time frame, this did not prevent assessment of weight on D1 of each cycle, which had to be recorded. The dose needed to be adjusted if change in body weight is >5% of weight at the previous cycle.

huMAb2-3-SPDB-DM4 was administered at 170 mg/m² or 150 mg/m² before ramucirumab, and then was administered at 100 mg/m².
Formulation: huMAb2-3-SPDB-DM4 was supplied as a 25 mL extractable volume of concentrate for solution for infusion of 125 mg contained in a 30 mL Type I glass vial.
Route of administration: IV infusion.
Dose regimen: huMAb2-3-SPDB-DM4 loading dose at 170 mg/m² or 150 mg/m² was administered via IV infusion over 1 hour 30 minutes on day 1 of Cycle 1, followed by 100 mg/m² every two weeks from Cycle 2 and in all other cycles.
For participants with a body surface area (BSA) >2.2 m², the dose was calculated based on a BSA of 2.2 m².

Using a controlled infusion pump, huMAb2-3-SPDB-DM4 was administered by IV infusion over 1 hour 30 minutes. For a participant with a BSA >2.2 m², the calculated dose of huMAb2-3-SPDB-DM4 was based on a BSA of 2.2 m².

Overview of study interventions administered.

TABLE 4
Overview of study interventions administered

| | | |
|---|---|---|
| Intervention label | huMAb2-3-SPDB-DM4 | Ramucirumab |
| Intervention name | huMAb2-3-SPDB-DM4 | ramucirumab |
| Type | Drug | Drug |
| Dose formulation | concentrated solution for IV | concentrated solution for IV |
| Unit dose strength(s) | 5 mg/ml | 10 mg/ml |
| Dosage levels^{a} | 170 (150) mg/m² Cycle 1 then 100 mg/m² | 8 mg/kg Q2W |
| | Q2W | |
| Route of administration | IV infusion | IV infusion |
| U | experimental | experimental |
| Use | | |
| IMP or NIMP | IMP | IMP |
| Packaging and labeling | Supplied in a 30 mL glass vial with a | Supplied in a single-dose vial |
| | white plastic flip-off cap, containing | (100 mg/10 mL or 500 mg/50 mL), |
| | 125 mg/25 mL huMAb2-3-SPDB-DM4, | labeled with a multilingual booklet, and |
| | and labelled with a multilingual booklet | individually packaged in a carton |
| Current/Former names or | huMAb2-3-SPDB-DM4 | Cyramza ® |
| aliases | | |

Abbreviations: IMP = investigational medicinal product; IV = intravenous; Q2W = every 2 weeks.
^{a}The huMAb2-3-SPDB-DM4 starting dose is 170 mg/m²; the dose may be decreased to 150 mg/m 2 (DL −1)

Premedication with an IV histamine-1 receptor antagonist (diphenhydramine 50 mg IV or equivalent; eg, cetirizine, promethazine, dexchlorpheniramine, according to local approval and availability) were given approximately at least 15 minutes before ramucirumab administration.

The Primary Objectives were to assess the tolerability and to confirm the recommended huMAb2-3-SPDB-DM4 loading dose Q2W when given in combination with ramucirumab in advanced gastric or gastroesophageal junction (GEJ) adenocarcinoma population and to assess the antitumor activity of huMAb2-3-SPDB-DM4 loading dose Q2W in combination with ramucirumab in advanced gastric or GEJ adenocarcinoma.

The Endpoints were the incidence of study drug related dose-limiting toxicities (DLTs) at Cycle 1 and Cycle 2 (C1D1 to C2D14) and the objective response rate (ORR), defined as the proportion of participants with confirmed complete response (CR) or partial response (PR) as best overall response (BOR) per Response Evaluation Criteria in Solid Tumors (RECIST) 1.1. The incidence of treatment-emergent adverse events (TEAEs), serious adverse events (SAEs), and laboratory abnormalities according to the National Cancer Institute (NCI) Common Terminology Criteria for Adverse Events (CTCAE) V5.0 was also recorded. All AEs from Table 5 occurring during the first 2 cycles of treatment, unless due to disease progression or to a cause obviously unrelated to IMP, were considered DLTs.

TABLE 5
Dose-limiting toxicities

Hematological abnormalities
Grade 4 neutropenia for 7 or more consecutive days.
Grade 3 to 4 neutropenia complicated by fever (temperature ≥38.5° C. on more than 1 occasion) or microbiologically or
radiographically documented infection
Grade ≥3 thrombocytopenia associated with clinically significant bleeding requiring clinical intervention
Nonhematological abnormalities
Grade 4 non-hematologic AE
Grade ≥3 keratopathy
In addition, any other AE that the recruiting Investigators and Sponsor deem to be dose limiting, regardless of its grade, may
also be considered as DLT.
Abbreviations: AE = adverse event; DLT = dose-limiting toxicity.

The assessment of antitumor activity of huMAb2-3-SPDB-DM4 combined with ramucirumab with regard to objective response rate (ORR) per RECIST 1.1 is the primary efficacy objective.

All participants treated must have at least one measurable lesion as per RECIST (Response Evaluation Criteria in Solid Tumors) 1.1 for inclusion based on tumor assessment. Tumors were assessed with chest, abdomen, or pelvic computed tomography (CT)-scan or Magnetic Resonance Imaging (MRI). Any other examinations as clinically indicated were performed to assess disease status at baseline and then every 6 weeks (±7 days) during the study treatment period until radiological disease progression; initiation of further anticancer therapy; death; or cut-off for secondary endpoints, whichever comes first. The scheduled tumor assessment time point will not be modified in case of a cycle delay. Brain CT-scan or MRI was performed at baseline only for known stable lesions or if clinically indicated and followed during treatment only for participants with brain lesions at baseline. Confirmatory radiological evaluation was performed at least 4 weeks after initial documentation of response.

### Results

The data were available for 24 patients.

TABLE
Objective response in gastric cancer (GC) or gastroesophageal
junction (GEJ) patients with high-CEACAM5 expression cancer
and treated with ramucirumab and tusamitamab ravtansine

| (CEACAM5 >=50%)* | | |
|---|---|---|
| « High expressors » | (N = 24) | |
| Objective response (CR + PR) | 3 | (13.5%) |
| Complete response | 0 | |
| Partial response | 3 | (13.5%) |
| Stable disease | 11 | (45.8%) |
| Progressive disease | 7 | (29%) |
| Not Evaluable | 3 | (13.6%) |
| Disease control rate (PR + SD) | 13 | (59%) |

*[IHC] intensity ≥2+ in ≥50% of cells

These data demonstrate that proof of concept was achieved in gastric cancer (GC) or GEJ patients with high-CEACAM5 expression cancer and treated with ramucirumab and tusamitamab ravtansine. In particular, these data support the conclusion that the combination of tusamitamab ravtansine with ramucirumab is effective in treating gastric or GEJ cancer. Moreover, these data support the conclusion that the combination of tusamitamab ravtansine with ramucirumab is particularly effective in treating high-CEACAM5 expressing gastric or GEJ cancers.

### Example 6: Antibody-Drug Conjugate huMAb2-3-SPDB-DM4 in Combination with the Ramucirumab in Pre-Treated Patients with Non-Squamous Non-Small-Cell Lung Cancer (NSQ NSCLC)

### Rationale

Despite recent progress in the treatment of advanced non-small-cell lung cancer (NSCLC), there remains a need for effective new treatment at the time of disease progression. Current therapeutic approaches combining an inhibitor of angiogenesis in combination with a systemic cytotoxic agent such as docetaxel entail serious haematological and other toxicities. New cytotoxic treatments selectively targeted to tumor cells have the potential to improve efficacy while managing toxicity. The aim of this study was to evaluate the safety and anti-tumor activity (efficacy) of tusamitamab ravtansine (huMAb2-3-SPDB-DM4) in combination with ramucirumab in patients with CEACAM5-positive (CEACAM5≥50%) NSQ NSCLC tumors.

One feature that can be used to target some tumor cells is surface expression of carcinoembryonic antigen-related cell adhesion molecule 5 (CEACAM5). High levels of CEACAM5 expression are observed in several epithelial tumors, including adenocarcinomas of the colon and stomach as well as NSQ NSCLC. Maytansinoids are antimitotic agents that inhibit microtubule formation to act as very potent cytotoxic agents against tumor cell lines in vitro, with IC 50 values 100- to 1000-fold more potent than conventional tubulin binding compounds, including docetaxel. huMAb2-3-SPDB-DM4 is an antibody to CEACAM5 conjugated to the cytotoxic maytansinoid agent, (DM4). Ramucirumab (Cyramza®), a human IgG₁ monoclonal antibody that inhibits vascular endothelial growth factor receptor-2 (VEGFR-2), administered 10 mg/kg every 3 weeks (Q3W) in combination with docetaxel, is approved for the treatment of patients with metastatic NSCLC.

### Experimental Procedure

An open-label, single-arm, multi-centers clinical trial was conducted to evaluate antitumor activity, safety, and pharmacokinetics of huMAb2-3-SPDB-DM4 used in combination with ramucirumab in metastatic, non-squamous non-small-cell lung cancer (NSQ NSCLC) patients with CEACAM5-positive tumors, defined as CEACAM5 immunohistochemical [IHC] intensity ≥2+ in ≥50% of cells, previously treated with platinum-based chemotherapy and an immune checkpoint inhibitor. Only participants with NSQ NSCLC determined to be CEACAM5 positive by central IHC went through protocol screening procedures during the screening phase.

The participants' inclusion criteria were as follows:

### Age

Participants must be 18 years of age (or country's legal age of majority, if >18 years), at the time of signing the informed consent.

### Type of Participant and Disease Characteristics

Histologically or cytologically proven diagnosis of NSQ NSCLC.

Metastatic disease progression fulfilling both of the following 2 criteria:
a) Having progressive disease during or after platinum-based chemotherapy (at least 2 cycles). Subsequent therapy following platinum-based chemotherapy is not considered as a separate regimen. Adjuvant/neoadjuvant treatment for a patient who had a relapse with metastatic disease during or within 6 months of completing treatment will be considered as first-line treatment.
AND
b) Having progressive disease during or after 1 immune checkpoint inhibitor (anti-PD1/PD-L1); this could be given as monotherapy or in combination with platinum-based chemotherapy (whatever the order).

For a tumor genotype with a sensitizing EGFR mutation or BRAF mutation or ALK/ROS alteration, demonstrated disease progression while receiving approved treatment for that genotype in addition to platinum-based chemotherapy and immune checkpoint inhibitor.

Expression of CEACAM5 as demonstrated prospectively by a centrally assessed immunohistochemical (IHC) assay of ≥2+ in intensity involving at least 50% of the tumor cell population in archival tumor sample (or if not available fresh biopsy sample). At least 5 slides of formalin-fixed, paraffin embedded (FFPE) tumor tissue sectioned at a thickness of 4 μm are required. If less material is available, the patient could still be considered eligible after discussion with the Sponsor, who may assess and confirm that the available material is sufficient for key evaluations.

At least one measurable lesion by RECIST v1.1 as determined by local site Investigator radiology assessment. An irradiated lesion can be considered measurable only if progression has been demonstrated on the irradiated lesion.

Eastern Cooperative Oncology Group (ECOG) performance status 0-1.

### Sex

All (male or female)

Contraceptive use by men or women should be consistent with local regulations regarding the methods of contraception for those participating in clinical studies.

### a) Male Participants

A male participant must agree to use contraception during the intervention period and for at least 4 months after the last dose of study intervention.

### b) Female Participants

A female participant is eligible to participate if she is not pregnant, not breastfeeding, and at least 1 of the following conditions applies:
Not a woman of child-bearing potential (WOCBP).

OR
A WOCBP who agrees to follow contraceptive guidance during the intervention period and for at least 7 months after the last dose of study intervention.

### Informed Consent

Capable of giving signed informed consent.

In the pre-screening phase, patients with NSQ NSCLC had tumor tissue tested centrally to assess proportions of CEACAM5-positive cells and intensity of expression of CEACAM5 expression on tumor tissue. For this analysis, at least 5×4 μm slides of formalin-fixed, paraffin embedded (FFPE) tumor tissue were analyzed according to a standard immunohistochemistry protocol. In brief, CEACAM5 tumor expression was assessed at prescreening on the most recent available archival tumor sample (i.e., archive tumor tissue at diagnosis, archive tumor tissue at surgery, or tumor sample before inclusion in the study and not under anticancer treatment). The level and pattern of CEACAM5 expression in tumor tissues were determined using IHC with an anti-CEACAM5 antibody run on Dako/Agilent Autostainer Link 48 IHC platform. Interpretation of CEACAM5 reactivity was performed by a board-certified pathologist using semi-quantitative Percent Scores (calculated by summing the percentages of intensities ≥2+) or H-score for CEACAM5 plasma membrane staining (whole or polarized) in tumor cells. Cytoplasmic staining was also evaluated.

Only participants with positive results, defined as CEACAM5 expression of ≥2+ in intensity involving at least 50% of the tumor cell population, in tumor sample entered the screening phase.

The Primary Objectives were to assess the tolerability and to confirm the recommended dose of huMAb2-3-SPDB-DM4 in combination with ramucirumab in the NSQ NSCLC population and the antitumor activity of huMAb2-3-SPDB-DM4 in combination with ramucirumab in the NSQ NSCLC population. The Endpoints were the incidence of study drug-related dose-limiting toxicity (DLT) at Cycle 1 and Cycle 2 (C1D1 to C2D14) and the objective response rate (ORR) defined as proportion of participants with confirmed complete response (CR) or partial response (PR) as best overall response (BOR) determined per Response Evaluation Criteria in Solid Tumors (RECIST) v1.1. Anticipated DLT included, but was not limited to, corneal toxicity. Also, the incidence of treatment emergent adverse events (TEAEs) and serious adverse events (SAEs) and laboratory abnormalities according to National Cancer Institute (NCI) Common Terminology Criteria for Adverse Events (CTCAE) V5.0 were recorded. All AEs from Table 6 occurring during the first 2 cycles of treatment, unless due to disease progression or to a cause obviously unrelated to IMP, were considered DLTs.

TABLE 6
Dose-limiting toxicities

Hematological abnormalities
Grade 4 neutropenia for 7 or more consecutive days.
Grade 3 to 4 neutropenia complicated by fever (temperature ≥38.5° C. on more than 1 occasion) or microbiologically or
radiographically documented infection
Grade ≥3 thrombocytopenia associated with clinically significant bleeding requiring clinical intervention
Non-hematological abnormalities
Elevated urine protein ≥3 g/24 h
Grade 4 non-hematologic AE
Grade ≥3 keratopathy
Grade 4 or refractory hypertension
In addition, any other AE that the recruiting Investigators and Sponsor deem to be dose limiting, regardless of its grade, may
also be considered as DLT.
Abbreviations:
AE: adverse event;
DLT: dose-limiting toxicity

The assessment of anti-tumor activity of huMAb2-3-SPDB-DM4 combined with ramucirumab is the primary efficacy objective.

All participants treated must have at least one measurable lesion as per RECIST v1.1 for inclusion based on tumor assessment. Tumor assessment were made every 8 weeks (±7 day window), and a scheduled assessment time point was not modified in case of a cycle delay. Thoracic-abdominal-pelvic computed tomography (CT)-scan or Magnetic Resonance Imaging (MRI). Any other examinations as clinically indicated were performed to assess disease status at baseline; then every 8 weeks during the study treatment period until radiological disease progression, initiation of further anticancer therapy, death, or study cut-off, whichever comes first; and at the end of study treatment, except if already done at last cycle. Confirmatory radiological evaluation was performed at least 4 weeks after initial documentation of response. After IMP discontinuation, tumor assessment was performed at EOT for patients without imaging performed within past 4 weeks, and every 12 weeks (±7 days) after the last tumor assessment until disease progression or initiation of a new anticancer treatment, death, or the study cut-off date, whichever comes first. Brain CT-scan or MRI was performed at baseline and followed only for patients with brain lesions at baseline. Imaging assessments during the on-treatment period were scheduled using the Cycle 1, day 1 date as the reference date for all time points, and were not to schedule based on the date of the previous imaging time point. The same tumor assessment technique was used throughout the study for a given lesion/participant. The RECIST v1.1 criteria was followed for assessment of tumor response.

This was a two-part study.

In Part 1 (Safety Run-In), participants received ramucirumab 8 mg/kg followed by huMAb2-3-SPDB-DM4 every 2 weeks to assess the tolerability of the combination to be used in the subsequent part of the study. The first 3 participants received ramucirumab at 8 mg/kg followed by huMAb2-3-SPDB-DM4 100 mg/m² every 2 weeks. Administration of huMAb2-3-SPDB-DM4 was initiated at least 1 hour after the completion of ramucirumab infusion (Table 7).

In case it was decided to reduce the initial loading dose of huMAb2-3-SPDB-DM4 to DL-1 (dose level −1), a huMAb2-3-SPDB-DM4 loading dose of 80 mg/m² was administered to participants on day 1 of Cycle 1 (Table 7).

The tolerability of the initial DL was assessed as follows: if ≥2 of the first 3 patients or of the 6 patients treated at the initial DL presented with DLTs, then it may be decided to decrease the dose of huMAb2-3-SPDB-DM4 to DL −1 (80 mg/m² in combination with 8 mg/kg ramucirumab). The tolerability of the reduced DL (DL-1) was assessed in at least 6 participants.

TABLE 7
Dose reduction for Phase 1 part (safety run-in)

| | | |
|---|---|---|
| Dose level (DL) | huMAb2-3-SPDB-DM4 | Ramucirumab |

| | | | |
|---|---|---|---|
| Starting dose | 100 | mg/m² Q2W | 8 mg/kg Q2W |
| Minus -1 (DL-1) | 80 | mg/m² Q2W | 8 mg/kg Q2W |

DL = dose level; Q2W = every 2 weeks.

Infusion of huMAb2-3-SPDB-DM4 was administered at least 1 hour after the end of ramucirumab infusion. For patients with a BSA >2.2 m², the huMAb2-3-SPDB-DM4 dose was calculated based on a BSA of 2.2 m².

In Part 2, 30 treated participants evaluable for response were planned (the 6 participants from the safety run-in treated at the RP2D were included.

The duration of the study for a participant included:
Screening period: up to 28 days.
Treatment period: once successfully screened, enrolled participants received study intervention until disease progression, unacceptable adverse effect (AE), or the participant's or investigator's decision to stop the treatment. Each cycle of treatment had a duration of 2 weeks. After discontinuing study intervention, participants returned to the study site approximately 30 days after the last investigational medicinal product (IMP) administration or before the participant received another anti-cancer therapy, whichever was earlier, for end-of-treatment assessments.
Safety follow-up visit was performed approximately 90 days after the last dose of IMP. If any ongoing related AE/SAE was resolved or stabilized, no further follow-up visit was needed.

### Investigational Medicinal Products (IMP)

Ramucirumab was administered prior to administration of huMAb2-3-SPDB-DM4 100 mg/m² or 80 mg/m².
Formulation: CYRAMZA® (ramucirumab) is a concentrate for solution for infusion supplied in 10 mL or 50 mL single-use vial. Each vial contains either 100 mg ramucirumab in 10 mL (10 mg/mL) or 500 mg ramucirumab in 50 mL (10 mg/mL).
Route of administration: intravenous (IV) infusion.
Dose regimen: ramucirumab was administered as an 8 mg/kg IV infusion over 1 hour on day 1 of every 2-week cycle. Each administration was preceded by premedication (with an IV histamine H1 antagonist as per the approved product label) to prevent hypersensitivity reaction.

huMAb2-3-SPDB-DM4 infusion started >1 hour after the end of ramucirumab infusion.
Formulation: huMAb2-3-SPDB-DM4 was supplied as a 25 mL extractable volume of concentrate for solution for infusion of 125 mg contained in a 30 mL type I glass vial.
Route of administration: IV infusion.
Dose regimen: huMAb2-3-SPDB-DM4 100 mg/m² (or 80 mg/m², if deemed the appropriate dose by the SC) was administered via IV infusion over 1 hour 30 minutes on day 1, and then every 2 weeks.
For patients with a body surface area (BSA) >2.2 m², the dose of huMAb2-3-SPDB-DM4 will be calculated based on a BSA of 2.2 m².

Overview of study interventions administered.

TABLE 8
Overview of study interventions administered

| Enter Arm Name (single arm) | huMAb2-3-SPDB-DM4 | ramucirumab |
|---|---|---|
| T Type | Drug/Biologic | Biologic |
| Dose formulation | Concentrated solution for IV | Concentrated solution for IV |
| Unit dose strength(s) | 5 mg/mL | 10 mg/mL |
| Dosage level(s) | 100 (80) mg/m² every 2 weeks | 8 mg/kg every 2 weeks |
| Route of administration | IV infusion | IV infusion |
| IMP (single-arm) | IMP | IMP |
| Packaging and labeling | Supplied in a 30 mL glass vial with a | Supplied in a single-dose vial |
| | white plastic flip-off cap, containing | (100 mg/10 mL or 500 mg/50 mL) |
| | 125 mg/25 mL huMAb2-3-SPDB-DM4, and | individually packaged in a carton. |
| | labelled with a multilingual booklet | |
| [Current/Former name(s) or alias(es)] | None | Cyramza |

Abbreviations: IMP = investigational medicinal product; IV = intravenous;

### Results

Table 9 below summarizes primary results on the primary objective of assessing the tolerability and confirming the recommended dose of huMAb2-3-SPDB-DM4 in combination with ramucirumab in the NSQ NSCLC population.

TABLE 9

| | huMAb2-3-SPDB-DM4 | |
|---|---|---|
| | 100 mg/m² | |
| | Ramucirumab 8 mg/kg | |
| | (N = 6) | |
| Participants with any TEAE | 6 | (100) |
| Participants with any grade ≥ 3 TEAE | 4 | (66.7) |
| Participants with any grade 5 TEAE | 2 | (33.3) |
| Participants with any treatment-emergent SAE | 2 | (33.3) |
| Participants with any treatment-emergent AESI | 1 | (16.7) |
| Participants with any TEAE leading to permanent full intervention discontinuation | 0 | |
| Participants with any TEAE leading to permanent discontinuation of tusamitamab ravtansine | 0 | |
| Participants with any TEAE leading to permanent discontinuation of ramucirumab | 0 | |
| Participants with any TEAE related to IMP | 4 | (66.7) |
| Participants with any grade ≥ 3 TEAE related to IMP | 3 | (50.0) |

Abbreviations: AESI: adverse event of special interest; SAE: serious adverse events; TEAE: treatment-emergent adverse event.

An AESI is an AE (serious or nonserious) of scientific and medical concern specific to the product or program, for which ongoing monitoring and immediate notification by the Investigator to the Sponsor is required. AESI considered during the study were, e.g., pregnancy; grade ≥3 keratopathy; bundle branch blocks or any conduction defects; grade ≥3 liver enzyme increased (symptomatic or asymptomatic); symptomatic overdose (serious or nonserious); all protocol-defined DLT.

The results show that no DLT was reported during cycle 1 and cycle 2 (DLT period observation) on the 6 first patients treated. Further, no treatment related serious adverse events (SAEs) were reported.

All patients experienced TEAEs; more frequent TEAEs all grades were hypertension and nausea in 3 participants (50%). Four patients experienced 7 TEAEs of grade >3 (2 hypertension, and 1 for each: pneumonia, keratopathy, hepatic function abnormal, asthenia, global health deterioration).

No Grade 3-4 anaemia, neutropenia or thrombocytopenia were reported. No Grade 3-4 creatinine increase and no Grade 3-4 AST/ALT increase were reported. One participant had proteinuria on dipstick +++ that recovered next cycles.

Two deaths on treatment due to Disease Progression (D32 and D38). No toxic death was reported.

2 patients have seen cycles delayed due to TEAE: keratopathy Grade 3 at cycles 6 and 10, and asthenia Grade 3 at cycle 1, and 1 patient has seen a dose of reduction huMAb2-3-SPDB-DM4 from cycle 11 due to keratopathy G3.

There was no dose interruption and no permanent discontinuation due to TEAEs.

In conclusion: the tolerability of recommended dose of huMAb2-3-SPDB-DM4 100 mg/m² and ramucirumab 8 mg/kg was confirmed.

The intermediary results on efficacy are shown in the following table.

The data were available for 31 patients.

TABLE
Objective response in non-squamous non-small-cell lung cancer
(NSQ NSCLC) patients with high-CEACAM5 expression cancer
and treated with ramucirumab and tusamitamab ravtansine

| (CEACAM5 ≥ 50%) | | |
|---|---|---|
| « High expressers » | (n = 31) | |
| Objective response (CR + PR) | 5 | (17.9%) |
| Complete response | 0 | |
| Partial response | 7 | (22.6%) |
| Stable disease | 17 | (60.7%) |
| Progressive disease | 5 | (17.9%) |
| Not Evaluable | 1 | (3.6%) |
| Disease control rate (PR + SD) | 22 | (78.6%) |

In conclusion, these data demonstrate that proof of concept was achieved in a subset of NSCLC lung cancers treated with the combination of tusamitamab ravtansine with ramucirumab. In particular, these data support the conclusion that the combination of tusamitamab ravtansine with ramucirumab is effective in treating NSQ NSCLC, a subtype that represents approximately 60% of lung cancers. Moreover, these data support the conclusion that the combination of tusamitamab ravtansine with ramucirumab is particularly effective in treating high CEACAM5 expressing NSQ NSCLC, a tumor types that represents approximately 20% of NSQ NSCLC cancers.

1. An antibody-drug conjugate comprising an anti-CEACAM5-antibody for use for treating cancer in combination with an anti-VEGFR2 antibody.

2. The antibody-drug conjugate for use of claim 1, wherein the anti-CEACAM5-antibody comprises a CDR-H1 consisting of SEQ ID NO: 1, CDR-H2 consisting of SEQ ID NO: 2, CDR-H3 consisting of SEQ ID NO: 3, CDR-L1 consisting of SEQ ID NO: 4, CDR-L2 consisting of amino acid sequence NTR, and CDR-L3 consisting of SEQ ID NO: 5.

3. The antibody-drug conjugate for use of claim 1 or 2, wherein the anti-CEACAM5-antibody comprises a variable domain of a heavy chain (VH) consisting of SEQ ID NO: 6 and a variable domain of a light chain (VL) consisting of SEQ ID NO: 7.

4. The antibody-drug conjugate for use of anyone of claims 1 to 3, wherein the anti-CEACAM5-antibody comprises a heavy chain (VH) consisting of SEQ ID NO: 8 and a light chain (VL) consisting of SEQ ID NO: 9.

5. The antibody-drug conjugate for the use of any of claims 1 to 4, wherein the antibody-drug conjugate comprises at least one chemotherapeutic agent.

6. The antibody-drug conjugate for the use of claim 5, wherein the chemotherapeutic agent is selected from the group consisting of radioisotopes, protein toxins, small molecule toxins, and combinations thereof.

7. The antibody-drug conjugate for the use of claim 6, wherein the small molecule toxins are selected from anti-metabolites, DNA-alkylating agents, DNA-cross-linking agents, DNA-intercalating agents, anti-microtubule agents, topoisomerase inhibitors, and combinations thereof.

8. The antibody-drug conjugate for the use of anyone of claims 5 to 7, wherein the chemotherapeutic agent is selected from the group consisting of taxanes, vinca alkaloids, maytansinoids, colchicine, podophyllotoxin, gruseofulvin, and combinations thereof.

9. The antibody-drug conjugate for the use of claim 8, wherein the maytansinoids are selected from the group consisting of N2′-deacetyl-N2′-(3-mercapto-1-oxopropyl)-maytansine (DM1) or N2′-deacetyl-N-2′(4-methyl-4-mercapto-1-oxopentyl)-maytansine (DM4), and combinations thereof.

10. The antibody-drug conjugate for the use of anyone of claims 5 to 9, wherein the anti-CEACAM5-antibody is covalently attached via a cleavable or non-cleavable linker to the at least one chemotherapeutic agent.

11. The antibody-drug conjugate for the use of claim 10, wherein said linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

12. The antibody-drug conjugate for the use according to anyone of claims 1 to 11, comprising an CEACAM5-antibody, which comprises a heavy chain (VH) consisting of SEQ ID NO: 8 and a light chain (VL) consisting of SEQ ID NO: 9 (huMAb2-3), and which is covalently linked to N2′-deacetyl-N-2′(4-methyl-4-mercapto-1-oxopentyl)-maytansine (DM4) via N-succinimidyl pyridyldithiobutyrate (SPDB).

13. The antibody-drug conjugate for the use of any of claims 1 to 12, wherein the antibody-drug conjugate and the anti-VEGFR2 antibody are formulated in a single pharmaceutical composition comprising the antibody-drug conjugate and the anti-VEGFR2 antibody.

14. The antibody-drug conjugate for the use of any of claims 1 to 12, wherein the antibody-drug conjugate and the anti-VEGFR2 antibody are formulated in the form of two separate pharmaceutical compositions, wherein (i) one pharmaceutical composition comprises the antibody-drug conjugate, and (ii) the other pharmaceutical composition comprises the anti-VEGFR2 antibody.

15. The antibody-drug conjugate for the use of claim 14, wherein the antibody-drug conjugate and the anti-VEGFR2 antibody are administered separately or sequentially to a patient in need thereof.

16. The antibody-drug conjugate for the use of claim 15, wherein the antibody-drug conjugate is administered after the anti-VEGFR-2 antibody.

17. The antibody-drug conjugate for the use of any of claims 1 to 16, wherein the antibody-drug conjugate and the anti-VEGFR-2 antibody are administered for at least one cycle of treatment.

18. The antibody-drug conjugate for the use of any of claims 1 to 17, wherein the antibody-drug conjugate and the anti-VEGFR-2 antibody are administered at day 1 of a first cycle of treatment.

19. The antibody-drug conjugate for the use any of claims 1 to 18, wherein the antibody-drug conjugate and the anti-VEGFR-2 antibody are administered at day 1 of at least one additional cycle of treatment.

20. The antibody-drug conjugate for the use of anyone of claims 17 to 19, wherein the cycle of treatment is 2 weeks or 3 weeks.

21. The antibody-drug conjugate for the use of anyone of claims 1 to 20, wherein the cancer is a CEACAM5-expressing cancer.

22. The antibody-drug conjugate for the use of any of claims 1 to 21, wherein the cancer is a CEACAM5 positive cancer having a CEACAM5 immunohistochemical intensity ≥2+ in ≥50% of cancer cells.

23. The antibody-drug conjugate for the use of any of claims 1 to 21, wherein the cancer is a CEACAM5 positive cancer having a CEACAM5 immunohistochemical intensity ≥2+ in ≥1% and <50% of cancer cells.

24. The antibody-drug conjugate for the use of anyone of claims 1 to 23, wherein the cancer is selected from hepatocellular carcinoma, colorectal cancer, gastric cancer, gastroesophageal junction (GEJ) adenocarcinoma, lung cancer, uterus cervix cancer, pancreas cancer, ovary cancer, thyroid cancer, bladder cancer, endometrium cancer, breast cancer, liver cancer (for instance cholangiocarcinoma), prostate cancer and skin cancer.

25. The antibody-drug conjugate for the use of any of claims 1 to 24, wherein the cancer is selected from gastric cancer, gastroesophageal junction (GEJ) adenocarcinoma, and lung cancer.

26. The antibody-drug conjugate for the use of any of claims 1 to 25, wherein the antibody-drug conjugate is administered at a dose of 60 mg/m² to 210 mg/m² and the anti-VEGFR-2 antibody is administered at a dose of 2 mg/kg to 20 mg/kg.

27. The antibody-drug conjugate for the use of anyone of claims 1 to 26, wherein the lung cancer is a non-squamous non-small-cell lung cancer.

28. The antibody-drug conjugate for the use of anyone of claims 1 to 27, wherein the antibody-drug conjugate is administered at a dose of 80 mg/m² to 170 mg/m².

29. The antibody-drug conjugate for the use of anyone of claims 18 to 28, wherein at day 1 of a first cycle of treatment the antibody-drug conjugate is administered at a dose of 80 mg/m², 100 mg/m², 120 mg/m², 135 mg/m², 150 mg/m² or 170 mg/m².

30. The antibody-drug conjugate for the use of anyone of claims 1 to 29, wherein the anti-VEGFR-2 antibody is administered at a dose from 8 mg/kg to 10 mg/kg.

31. The antibody-drug conjugate for the use of anyone of claims 18 to 30, wherein at day 1 of a first cycle of treatment the antibody-drug conjugate is administered at a dose of 80 mg/m², 100 mg/m², 150 mg/m² or 170 mg/m², and the cycle of treatment is 2 weeks.

32. The antibody-drug conjugate for the use of anyone of claims 18 to 30, wherein at day 1 of a first cycle of treatment the antibody-drug conjugate is administered at a dose of 80 mg/m², 100 mg/m², 120 mg/m², 135 mg/m², 150 mg/m² or 170 mg/m², and the cycle of treatment is 3 weeks.

33. The antibody-drug conjugate for the use of claim 32, wherein at day 1 of a first cycle of treatment the antibody-drug conjugate is administered at a dose of 120 mg/m², 135 mg/m², 150 mg/m² or 170 mg/m², and the cycle of treatment is 3 weeks.

34. The antibody-drug conjugate for the use of anyone of claims 1 to 32, wherein the anti-VEGFR-2 antibody is administered at a dose from 8 mg/kg, and the cycle of treatment is 2 weeks.

35. The antibody-drug conjugate for the use of anyone of claims 1 to 31, and 33, wherein the anti-VEGFR-2 antibody is administered at a dose from 10 mg/kg, and the cycle of treatment is 3 weeks.

36. The antibody-drug conjugate for the use of anyone of claims 1 to 35, wherein the cancer is a lung cancer and wherein the antibody-drug conjugate is administered at a dose of 80 mg/m² to 170 mg/m².

37. The antibody-drug conjugate for the use of claim 36, wherein at day 1 of a first cycle of treatment the antibody-drug conjugate is administered at a dose of 80 mg/m², 100 mg/m², 120 mg/m² or 150 mg/m².

38. The antibody-drug conjugate for the use of claim 36 or 37, wherein at day 1 of a first cycle of treatment the antibody-drug conjugate is administered at a dose of 80 mg/m² or 100 mg/m², and the cycle of treatment is 2 weeks.

39. The antibody-drug conjugate for the use of anyone of claims 36 to 38, wherein at day 1 of a first cycle of treatment the anti-VEGFR-2 antibody is administered at a dose of 8 mg/kg, and the cycle is 2 weeks.

40. The antibody-drug conjugate for the use of claim 36 or 37, wherein at day 1 of a first cycle of treatment the antibody-drug conjugate is administered at a dose of 120 mg/m² or 150 mg/m², and the cycle of treatment is 3 weeks.

41. The antibody-drug conjugate for the use of anyone of claims 36, 37 and 40, wherein at day 1 of a first cycle of treatment the anti-VEGFR-2 antibody is administered at a dose of 10 mg/kg, and the cycle is 3 weeks.

42. The antibody-drug conjugate for the use of anyone of claims 36 to 41, wherein at day 1 of an at least one additional cycle of treatment the antibody-drug conjugate is administered at a dose of 80 mg/m², 100 mg/m², 120 mg/m² or 150 mg/m².

43. The antibody-drug conjugate for the use of anyone of claims 36 to 39, 41 and 42, wherein at day 1 of an at least one additional cycle of treatment the antibody-drug conjugate is administered at a dose of 80 mg/m² or 100 mg/m², and the cycle is 2 weeks.

44. The antibody-drug conjugate for the use of anyone of claims 36 to 39 and 42 to 43, wherein at day 1 of an at least one additional cycle of treatment the anti-VEGFR-2 antibody is administered at a dose of 8 mg/kg, and the cycle is 2 weeks.

45. The antibody-drug conjugate for the use of anyone of claims 36, 37 and 40 to 42, wherein at day 1 of an at least one additional cycle of treatment the antibody-drug conjugate is administered at a dose of 120 mg/m² or 150 mg/m², and the cycle last 3 weeks.

46. The antibody-drug conjugate for the use of anyone of claims 36, 37, 40 to 42, and 45, wherein at day 1 of an at least one additional cycle of treatment the anti-VEGFR-2 antibody is administered at a dose of 10 mg/kg, and the cycle is 3 weeks.

47. The antibody-drug conjugate for the use of anyone of claims 36 to 39 and 42 to 44, wherein at day 1 of a first cycle and at day 1 of an at least one additional cycle of treatment, the antibody-drug conjugate is administered at a dose of 80 mg/m² and the anti-VEGFR-2 antibody is administered at a dose of 8 mg/kg, and the cycle of treatment is 2 weeks.

48. The antibody-drug conjugate for the use of anyone of claims 36 to 39 and 42 to 44, wherein at day 1 of a first cycle and at day 1 of an at least one additional cycle of treatment, the antibody-drug conjugate is administered at a dose of 100 mg/m² and the anti-VEGFR-2 antibody is administered at a dose of 8 mg/kg, and the cycle of treatment is 2 weeks.

49. The antibody-drug conjugate for the use of anyone of claims 36, 37, 40 to 42, 45 and 46, wherein at day 1 of a first cycle and at day 1 of an at least one additional cycle of treatment, the antibody-drug conjugate is administered at a dose of 120 mg/m² and the anti-VEGFR-2 antibody is administered at a dose of 10 mg/kg, and the cycle of treatment is 3 weeks.

50. The antibody-drug conjugate for the use of anyone of claims 36, 37, 40 to 42, 45 and 46, wherein at day 1 of a first cycle and at day 1 of an at least one additional cycle of treatment, the antibody-drug conjugate is administered at a dose of 150 mg/m² and the anti-VEGFR-2 antibody is administered at a dose of 10 mg/kg, and the cycle of treatment is 3 weeks.

51. The antibody-drug conjugate for the use of any of claims 36 to 39 and 42 to 44, and 47, wherein the cancer is a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg and subsequently the antibody-drug conjugate comprising an anti-CEACAM5-antibody is administered at a dose of 80 mg/m², on day 1 of a first cycle, and the cycle is 2 weeks.

52. The antibody-drug conjugate for the use of claim 51, wherein the anti-VEGFR-2 antibody is administered at a dose of 8 mg/kg as a subsequent dose, and subsequently the antibody-drug conjugate comprising an anti-CEACAM5-antibody is administered at a dose of 80 mg/m² as a subsequent dose, on day 1 of additional cycle(s), and the cycle(s) is/are 2 weeks.

53. The antibody-drug conjugate for the use of any of claims 36 to 39 and 42 to 44, and 48, wherein the cancer is a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg and subsequently the antibody-drug conjugate comprising an anti-CEACAM5-antibody is administered at a dose of 100 mg/m², on day 1 of a first cycle, and the cycle is 2 weeks.

54. The antibody-drug conjugate for the use of claim 53, wherein the anti-VEGFR-2 antibody is administered at a dose of 8 mg/kg as a subsequent dose, and subsequently the antibody-drug conjugate comprising an anti-CEACAM5-antibody is administered at a dose of 100 mg/m² as a subsequent dose, on day 1 of additional cycle(s), and the cycle(s) is/are 2 weeks.

55. The antibody-drug conjugate for the use of any of claims 36, 37, 40 to 42, 45, 46 and 49, wherein the cancer is a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg and subsequently the antibody-drug conjugate comprising an anti-CEACAM5-antibody is administered at a dose of 120 mg/m², on day 1 of a first cycle, and the cycle is 3 weeks.

56. The antibody-drug conjugate for the use of claim 55, wherein the anti-VEGFR-2 antibody is administered at a dose of 10 mg/kg as a subsequent dose, and sequentially the antibody-drug conjugate comprising an anti-CEACAM5-antibody is administered at a dose of 120 mg/m² as a subsequent dose, on day 1 of additional cycle(s), and the cycle is 3 weeks.

57. The antibody-drug conjugate for the use of any of claims 36, 37, 40 to 42, 45, 46, and 50, wherein the cancer is a lung cancer in patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg and subsequently the antibody-drug conjugate comprising an anti-CEACAM5-antibody is administered at a dose of 150 mg/m², on day 1 of a first cycle, and the cycle is 3 weeks.

58. The antibody-drug conjugate for the use of claim 57, wherein the anti-VEGFR-2 antibody is administered at a dose of 10 mg/kg as a subsequent dose, and sequentially the antibody-drug conjugate comprising an anti-CEACAM5-antibody is administered at a dose of 150 mg/m² as a subsequent dose, on day 1 of additional cycle(s), and the cycle is 3 weeks.

59. The antibody-drug conjugate for the use of anyone of claims 1 to 25, wherein the cancer is a gastric cancer or a gastroesophageal adenocarcinoma (GEJ) cancer and wherein the antibody-drug conjugate is administered at a dose of 80 mg/m² to 170 mg/m².

60. The antibody-drug conjugate for the use of claim 59, wherein at day 1 of a first cycle of treatment the antibody-drug conjugate is administered at a dose of 80 mg/m², 100 mg/m², 120 mg/m², 135 mg/m², 150 mg/m² or 170 mg/m².

61. The antibody-drug conjugate for the use of claim 59 or 60, wherein at day 1 of a first cycle of treatment the antibody-drug conjugate is administered at a loading dose of 120 mg/m², 135 mg/m², 150 mg/m² or 170 mg/m², and the cycle is 2 weeks.

62. The antibody-drug conjugate for the use of claim 61, wherein at day 1 of a first cycle of treatment the antibody-drug conjugate is administered at a loading dose of 150 mg/m² or 170 mg/m², and the cycle is 2 weeks.

63. The antibody-drug conjugate for the of claim 61 or 62, wherein at day 1 of an at least one additional cycle of treatment the antibody-drug conjugate is administered at a subsequent dose of 80 mg/m² or 100 mg/m², and the cycle is 2 weeks.

64. The antibody-drug conjugate for the use of anyone of claims 59 to 63, wherein the anti-VEGFR2 antibody is administered at a dose of 8 mg/kg, and the cycle is 2 weeks.

65. The antibody-drug conjugate for the use of anyone of claims 59 to 64, wherein at day 1 of a first cycle, the antibody-drug conjugate is administered at a loading dose of 150 mg/m² and the anti-VEGFR-2 antibody is administered at a dose of 8 mg/kg, and at day 1 of an at least one additional cycle of treatment the antibody-drug conjugate is administered at a subsequent dose of 80 mg/m² and the anti-VEGFR-2 antibody is administered at a dose of 8 mg/kg, and the cycle of treatment is 2 weeks.

66. The antibody-drug conjugate for the use of anyone of claims 59 to 64, wherein at day 1 of a first cycle, the antibody-drug conjugate is administered at a loading dose of 170 mg/m² and the anti-VEGFR-2 antibody is administered at a dose of 8 mg/kg, and at day 1 of an at least one additional cycle of treatment the antibody-drug conjugate is administered at a subsequent dose of 100 mg/m² and the anti-VEGFR-2 antibody is administered at a dose of 8 mg/kg, and the cycle of treatment is 2 weeks.

67. The antibody-drug conjugate for the use of any of claims 59 to 65, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg, and subsequently the antibody-drug conjugate comprising an anti-CEACAM5-antibody is administered at a dose of 150 mg/m², on day 1 of a first cycle, and the cycle is 2 weeks.

68. The antibody-drug conjugate for the use of claim 67, wherein the anti-VEGFR-2 antibody is administered at a dose of 8 mg/kg as a subsequent dose, and subsequently the antibody-drug conjugate comprising an anti-CEACAM5-antibody is administered at a dose of 80 mg/m² as a subsequent dose, on day 1 of additional cycle(s), and the cycle(s) is/are 2 weeks.

69. The antibody-drug conjugate for the use of any of claims 60 to 64 and 66, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 8 mg/kg, and subsequently the antibody-drug conjugate comprising an anti-CEACAM5-antibody is administered at a dose of 170 mg/m², on day 1 of a first cycle, and the cycle is 2 weeks.

70. The antibody-drug conjugate for the use of claim 69, wherein the anti-VEGFR-2 antibody is administered at a dose of 8 mg/kg as a subsequent dose, and subsequently the antibody-drug conjugate comprising an anti-CEACAM5-antibody is administered at a dose of 100 mg/m² as a subsequent dose, on day 1 of additional cycle(s), and the cycle(s) is/are 2 weeks.

71. The antibody-drug conjugate for the use of claim 59 or 60, wherein the antibody-drug conjugate is administered at a dose of 80 mg/m², 100 mg/m², 120 mg/m², 135 mg/m² 150 mg/m² or 170 mg/m², and the cycle is 3 weeks.

72. The antibody-drug conjugate for the use of anyone of claims 59, 60 and 71, wherein the antibody-drug conjugate is administered at a dose of 120 mg/m², 150 mg/m² or 170 mg/m², and the cycle is 3 weeks.

73. The antibody-drug conjugate for the use of anyone of claims 59, 60, 71 and 72, wherein the anti-VEGFR2 antibody is administered at a dose of 10 mg/kg, and the cycle is 3 weeks.

74. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 80 mg/m² and the anti-VEGFR-2 antibody is administered at a dose of 10 mg/kg, and the cycle of treatment is 3 weeks.

75. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 100 mg/m² and the anti-VEGFR-2 antibody is administered at a dose of 10 mg/kg, and the cycle of treatment is 3 weeks.

76. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 120 mg/m² and the anti-VEGFR-2 antibody is administered at a dose of 10 mg/kg, and the cycle of treatment is 3 weeks.

77. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 135 mg/m² and the anti-VEGFR-2 antibody is administered at a dose of 10 mg/kg, and the cycle of treatment is 3 weeks.

78. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 150 mg/m² and the anti-VEGFR-2 antibody is administered at a dose of 10 mg/kg, and the cycle of treatment is 3 weeks.

79. The antibody-drug conjugate for the use of anyone of claims 59, 60 and 71 to 73, wherein at day 1 of a first cycle and at day 1 of an at least one additional cycle of treatment, the antibody-drug conjugate is administered at a dose of 170 mg/m² and the anti-VEGFR-2 antibody is administered at a dose of 10 mg/kg, and the cycle of treatment is 3 weeks.

80. The antibody-drug conjugate for the use of any of claims 59, 60 and 71 to 79, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 80 mg/m², 100 mg/m², 120 mg/m², 150 mg/m² or 170 mg/m², on day 1 of a first cycle, and the cycle is 3 weeks.

81. The antibody-drug conjugate for the use of claim 80, wherein the anti-VEGFR-2 antibody is administered at a dose of 10 mg/kg as a subsequent dose, and subsequently the antibody-drug conjugate comprising an anti-CEACAM5-antibody is administered at a dose of 80 mg/m², 100 mg/m², 120 mg/m², 150 mg/m² or 170 mg/m² as a subsequent dose, on day 1 of additional cycle(s), and the cycle(s) is/are 3 weeks.

82. The antibody-drug conjugate for the use of any of claims 59, 60 and 71 to 74, and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of c cancer ells, wherein the anti-VEGFR-2 antibody is administered at a dose of 10 mg/kg, and subsequently the antibody-drug conjugate comprising an anti-CEACAM5-antibody is administered at a dose of 80 mg/m², on day 1 of a first cycle, and the cycle is 3 weeks.

83. The antibody-drug conjugate for the use of claim 82, wherein the anti-VEGFR-2 antibody is administered at a dose of 10 mg/kg as a subsequent dose, and subsequently the antibody-drug conjugate comprising an anti-CEACAM5-antibody is administered at a dose of 80 mg/m² as a subsequent dose, on day 1 of additional cycle(s), and the cycle(s) is/are 3 weeks.

84. The antibody-drug conjugate for the use of any of claims 59, 60 and 71 to 73, 75 and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 100 mg/m², on day 1 of a first cycle, and the cycle is 3 weeks.

85. The antibody-drug conjugate for the use of claim 84, wherein the anti-VEGFR-2 antibody is administered at a dose of 10 mg/kg as a subsequent dose, and subsequently the antibody-drug conjugate comprising an anti-CEACAM5-antibody is administered at a dose of 100 mg/m² as a subsequent dose, on day 1 of additional cycle(s), and the cycle(s) is/are 3 weeks.

86. The antibody-drug conjugate for the use of any of claims 59, 60 and 71 to 73, 76 and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 120 mg/m², on day 1 of a first cycle, and the cycle is 3 weeks.

87. The antibody-drug conjugate for the use of claim 86, wherein the anti-VEGFR-2 antibody is administered at a dose of 10 mg/kg as a subsequent dose, and subsequently the antibody-drug conjugate comprising an anti-CEACAM5-antibody is administered at a dose of 120 mg/m² as a subsequent dose, on day 1 of additional cycle(s), and the cycle(s) is/are 3 weeks.

88. The antibody-drug conjugate for the use of any of claims 59, 60 and 71 to 73, 77 and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 135 mg/m², on day 1 of a first cycle, and the cycle is 3 weeks.

89. The antibody-drug conjugate for the use of claim 87, wherein the anti-VEGFR-2 antibody is administered at a dose of 10 mg/kg as a subsequent dose, and subsequently the antibody-drug conjugate comprising an anti-CEACAM5-antibody is administered at a dose of 135 mg/m² as a subsequent dose, on day 1 of additional cycle(s), and the cycle(s) is/are 3 weeks.

90. The antibody-drug conjugate for the use of any of claims 59, 60 and 71 to 73, 78 and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 150 mg/m², on day 1 of a first cycle, and the cycle is 3 weeks.

91. The antibody-drug conjugate for the use of claim 90, wherein the anti-VEGFR-2 antibody is administered at a dose of 10 mg/kg as a subsequent dose, and subsequently the antibody-drug conjugate comprising an anti-CEACAM5-antibody is administered at a dose of 150 mg/m² as a subsequent dose, on day 1 of additional cycle(s), and the cycle(s) is/are 3 weeks.

92. The antibody-drug conjugate for the use of any of claims 59, 60 and 71 to 73, 79 and 80, wherein the cancer is a gastric cancer (GC) or a gastroesophageal adenocarcinoma (GEJ) cancer in a patient, wherein the patient has a CEACAM5-positive cancer having a CEACAM5 immunohistochemical intensity ≥2+ in ≥50% of cancer cells or a CEACAM5 immunohistochemical intensity ≥2+ in ≥1% and <50% of cancer cells, wherein the anti-VEGFR-2 antibody is administered at a dose of 10 mg/kg, and subsequently the antibody-drug conjugate comprising an anti-CEACAM5-antibody is administered at a dose of 170 mg/m², on day 1 of a first cycle, and the cycle is 3 weeks.

93. The antibody-drug conjugate for the use of claim 92, wherein the anti-VEGFR-2 antibody is administered at a dose of 10 mg/kg as a subsequent dose, and subsequently the antibody-drug conjugate comprising an anti-CEACAM5-antibody is administered at a dose of 170 mg/m² as a subsequent dose, on day 1 of additional cycle(s), and the cycle(s) is/are 3 weeks.

94. The antibody-drug conjugate for the use according to anyone of claims 1 to 93, wherein the antibody-drug conjugate is tusamitamab ravtansine.

95. The antibody-drug conjugate for the use according to anyone of claims 1 to 94, wherein the anti-VEGFR-2 antibody is ramucirumab.

96. A pharmaceutical composition comprising the antibody-drug conjugate of anyone of claims 1 to 12, and an anti-VEGFR2 antibody, and a pharmaceutically acceptable excipient.

97. A pharmaceutical composition comprising the antibody-drug conjugate of anyone of claims 1 to 12, and ramucirumab and a pharmaceutically acceptable excipient.

98. A pharmaceutical composition comprising tusamitamab ravtansine, and ramucirumab and a pharmaceutically acceptable excipient.

99. A kit comprising (i) a pharmaceutical composition of the antibody-drug conjugate of anyone of claims 1 to 12 and a pharmaceutically acceptable excipient and (ii) a pharmaceutical composition comprising an anti-VEGFR2 antibody and a pharmaceutically acceptable excipient.

100. The pharmaceutical composition according to anyone of claims 96 to 98, or the kit according to claim 99, for the use for treating cancer.
